(19) United States
(12) Patent Application Publication
(10) Pub. No.: US 2012/0039939 A1
(43) Pub. Date: Feb. 16, 2012

(54) COMPOSITIONS AND METHODS FOR VACCINE AND VIRUS PRODUCTION

(75) Inventors: Joseph Shiloach, Rockville, MD (US); Michael Betenbaugh, Baltimore, MD (US); Pratik Jaluria, Chevy Chase, MD (US); Chia Chu, North Bethesda, MD (US)

(73) Assignee: THE JOHNS HOPKINS UNIVERSITY, Baltimore, MD (US)

(21) Appl. No.: 12/937,185

(22) PCT Filed: Apr. 10, 2009
(86) PCT No.: PCT/US09/02252

§ 371 Date: Oct. 8, 2010

Related U.S. Application Data
(60) Provisional application No. 61/124,077, filed on Apr. 11, 2008.

(51) Int. Cl. A61K 39/235; C12N 7/02; A61P 31/14; A61K 39/12; A61P 37/04

(52) U.S. Cl. 424/233.1; 424/204.1; 435/239

(57) ABSTRACT

The present invention features methods of producing immunogenic compositions and viruses, methods of treating and preventing viral infection, and methods of producing an immune response using cells that express a polypeptide selected from the group consisting of: cdk13, siat7e, Iama4, cox15, egr1, gas6, map3k9, and gap43, and a virus.

### CROSS-REFERENCE TO RELATED APPLICATIONS

This application claims priority to U.S. Provisional Application No. 61/124,077, filed on Apr. 11, 2008, the entire contents of which is hereby incorporated in its entirety. This application is related to PCT Application No. PCT/US2007/018699, which was filed on Aug. 24, 2007, U.S. Provisional Application No. 60/931,439, which was filed on May 23, 2007, and 60/840,381, which was filed on Aug. 24, 2006, the entire disclosures of which are hereby incorporated in their entireties.

### INCORPORATION BY REFERENCE

Each of the applications and patents cited in this text, as well as each document or reference cited in each of the applications and patents (including during the prosecution of each issued patent; “application cited documents”), and each of the PCT and foreign applications or patents corresponding to and/or paragraphing priority from any of these applications and patents, and each of the documents cited or referenced in each of the application cited documents, are hereby expressly incorporated herein by reference. More generally, documents or references are cited in this text, either in a Reference List, or in the text itself; and, each of these documents or references (“herein-cited references”), as well as each document or reference cited in each of the herein-cited references (including any manufacturer's specifications, instructions, etc.), is hereby expressly incorporated herein by reference.

### STATEMENT OF RIGHTS TO INVENTIONS MADE UNDER FEDERALLY SPONSORED RESEARCH

Research supporting this application was carried out by the United States of America as represented by the Secretary, Department of Health and Human Services. The Government may have certain rights in this invention.

### BACKGROUND OF THE INVENTION

Influenza-related illnesses cause an estimated 100,000 hospitalizations and tens of thousands of deaths in the United States annually. In response to rapid antigenic drift in influenza viruses, the most effective approach taken has been the distribution of trivalent inactivated viral vaccines, which are traditionally produced in chicken embryonated eggs. The vaccines confer protection against infection and disease by stimulating the production of immune responses to the hemagglutinin (HA), neuraminidase (NA), nucleoproteins (NP, and possibly other proteins of component strains. In the event of a pandemic outbreak, this egg-based production system may not be adequate to meet the surge in demand quickly enough.

Worldwide several hundred million of eggs are used each year to produce vaccine for the influenza season. The current production cycle (beginning with identification of the anticipated virus strains expected to be present in the forthcoming influenza season) is many months long. The current production processes that use fertile eggs as tiny bioreactors is labor intensive, expensive and fraught with variables, such as the seasonal availability and variation of properties of the eggs.

The limitations associated with egg-based vaccines, which include reliable egg supplies, prolonged cultivation periods, and cumbersome operations have spurred exploration of alternatives. Among the potential alternatives for vaccine production, the use of characterized, immortalized cell lines (particularly VERO, PERC6, and MDCK) has been investigated. These cell lines have been found to consistently produce high viral titers in a commercially viable manner. Nevertheless, one of the limiting aspects in scaling up the virus production in these continuous cell lines is the fact that these cells are anchorage-dependent and thus require surface adhesion in order to proliferate. Without surface attachment, these cells can not exert their normal cyclin-dependent kinase activity through the signaling cascades initialized by interactions between integrins and extracellular matrix. For industrial production in bioreactors, the required surface area can be provided by using microcarrier beads. Although this approach is sufficient to obtain high virus production yield, this propagation strategy is cumbersome compared with propagation of cells in suspension. An MDCK cell line that can proliferate in suspension would greatly faciliate the scale-up process of influenza virus production.

It would therefore be desirable to provide improved virus vaccine preparations that do not exhibit as many of the limitations and drawbacks observed with the use of currently available vaccines.

### SUMMARY OF THE INVENTION

As described below, the present invention features methods of producing immunogenic compositions and viruses, methods of treating and preventing viral infection, and methods of producing an immune response using cells that express a polypeptide or an inhibitory nucleic acid molecule that a sialyltransferase or a laminin, and in particular embodiments, is any one or more of cdk13, siat7e, lama4, cox15, egr1, gas6, map3k9, and gap43, and a virus.

In one aspect, the invention provides a method of producing an virus comprising a polynucleotide encoding a recombinant polypeptide, the method comprising isolating a virus from a virus infected cell, the cell comprising an expression vector comprising a nucleic acid molecule encoding a polypeptide corresponding to a sialyltransferase, thereby producing a virus comprising a polynucleotide encoding a recombinant polypeptide.

In certain embodiments, the sialyltransferase is selected from the group consisting of: siat1, siat2, siat3, siat4A, siat4B, siat4C, siat5, siat6, siat7, siat7D, siat7E, siat8A, siat8B, siat8C, siat8D, siat8E, siat9, and siatL. In further embodiments, the sialyltransferase is siat7e.

In another aspect, the invention provides a method of producing a virus comprising a polynucleotide encoding a recombinant polypeptide, the method comprising isolating a virus from a virus infected cell, the cell comprising an expression vector comprising a nucleic acid molecule encoding a polypeptide corresponding to a laminin glycoprotein, thereby producing a virus comprising a polynucleotide encoding a recombinant polypeptide.

In one embodiment, the laminin is lama4.

In another aspect, the invention provides a cell containing an expression vector containing a nucleic acid molecule encoding a polypeptide or an inhibitory nucleic acid molecule that is a sialyltransferase, and a virus (e.g., influenza virus, pneumovirus, hoof in mouth disease, and varicella zoster).

In certain embodiments, the sialyltransferase is elected from the group consisting of: siat1, siat2, siat3, siat4A, siat4B, siat4C, siat5, siat6, siat7, siat7D, siat7E, siat8A, siat8B, siat8C, siat8D, siat8E, siat9, and siatL. In further embodiments, the sialyltransferase is siat7e.

In another aspect, the invention provides a cell containing an expression vector containing a nucleic acid molecule encoding a polypeptide or an inhibitory nucleic acid molecule that is a laminin, and a virus (e.g., influenza virus, pneumovirus, hoof in mouth disease, and varicella zoster).

In one embodiment, the laminin is lama4.

In one aspect, the invention provides a cell containing an expression vector containing a nucleic acid molecule encoding a polypeptide or an inhibitory nucleic acid molecule that is any one or more of cdk13, siat7e, lama4, cox15, egr1, gas6, map3k9, and gap43, and a virus (e.g., influenza virus, pneumovirus, hoof in mouth disease, and varicella zoster). In one embodiment, the influenza virus is a human, avian, or canine influenza virus. In another embodiment, an adenovirus.

In a related aspect, the invention features a cell containing a mutation that alters the expression or activity of a polypeptide that is any one or more of cdk13, siat7e, lama4, cox15, egr1, gas6, map3k9, and gap43 polypeptide, and a virus. In one embodiment, the mutation is a deletion, missense mutation, or frameshift.

In another aspect, the invention features a method of producing an virus containing a polynucleotide encoding a recombinant polypeptide, the method involving isolating a virus from a virus infected cell, the cell containing an expression vector containing a nucleic acid molecule encoding a polypeptide that is any one or more of cdk13, siat7e, lama4, cox15, egr1, gas6, map3k9, and gap43, thereby producing a virus containing a polynucleotide encoding a recombinant polypeptide.

In another aspect, the invention features a method of producing an immunogenic composition containing a virus, the method involving isolating a virus from a virus infected cell, the cell containing an expression vector containing a nucleic acid molecule encoding a polypeptide that is any one or more of cdk13, siat7e, lama4, cox15, egr1, gas6, map3k9, and gap43; thereby producing an immunogenic composition containing a virus. In one embodiment, the method further involves the step of inactivating the virus. In another embodiment, the inactivation is heat inactivation.

In another aspect, the invention features a virus produced according to the method of any one of the previous claims.

In another aspect, the invention features a method of producing a vaccine or immunogenic composition, the method involving isolating a virus from the cell of any previous claim, and incorporating an effective amount of the virus into a pharmaceutically acceptable excipient.

In yet another aspect, the invention features a method of producing a vaccine or an immunogenic composition in a cell, the method involves infecting a cell containing an expression vector containing a nucleic acid molecule encoding a polypeptide selected from the group consisting of: cdk13, siat7e, lama4, cox15, egr1, gas6, map3k9, and gap43 with a virus; producing virus in the cell; and harvesting the virus; thereby producing a vaccine in the cell.

In another aspect, the invention features a method of producing a vaccine or an immunogenic composition in a cell, the method involving infecting a cell containing an expression vector containing a nucleic acid molecule encoding a siat7e, lama4, cdk13, cox15, egr1, or gas6 inhibitory nucleic acid molecule with a virus; producing virus in the cell; and harvesting the virus; thereby producing an immunogenic composition in the cell.

In yet another aspect, the invention features a method of producing a vaccine or an immunogenic composition in a cell, the method involving infecting a cell, wherein the cell comprises a mutation that alters the expression or activity of a polypeptide selected from the group consisting of cdk13, siat7e, lama4, cox15, egr1, gas6, map3k9, and gap43 polypeptide with a virus; producing virus in the cell; and harvesting the virus; thereby producing a virus or an immunogenic composition in the cell.

In another aspect, the invention features a immunogenic composition produced by the method of any previous claim in a pharmaceutically acceptable carrier. In one embodiment, the composition is capable of generating a protective immune response to a virus or pathogen when administered to a mammal.

In another aspect, the invention features a vaccine produced by the method of any previous claim. In one embodiment, the vaccine is capable of generating an immune response against a virus selected from the group consisting of: influenza virus, pneumovirus, hoof in mouth disease, and varicella zoster. In another embodiment, the influenza virus is selected from the group consisting of: human, avian, and canine influenza virus.

In a related aspect, the invention features a virus produced by the method of any previous aspect in a pharmaceutically acceptable carrier.

In another aspect, the invention features a method of producing an immune response in a subject, the method involving administering to the subject the pharmaceutical composition of a previous aspect in an amount sufficient to generate an immune response, thereby producing an immune response in a subject.

In another aspect, the invention features a method of treating a subject suffering from a viral infection, the method involving administering to the subject the pharmaceutical composition a previous aspect in an amount sufficient to generate an immune response, thereby treating a subject suffering from a viral infection.

In a related aspect, the invention features a method of preventing a viral infection in a subject, the method involving administering to the subject the pharmaceutical composition of a previous aspect in an amount sufficient to generate an immune response, thereby preventing a viral infection in a subject. In one embodiment, the mode of administration is topical administration, oral administration, injection by needle, needleless jet injection, intradermal administration, intramuscular administration, or gene gun administration. In another embodiment, n the immune response is a protective immune response. In another embodiment, the immune response is a cell-mediated immune response. In another embodiment, the immune response is a humoral immune response. In yet another embodiment, wherein the immune response is a cell-mediated immune response and a humoral immune response.

In various embodiments of the previous aspects, the method further involves isolating immune cells from the subject; and testing an immune response of the isolated immune cells in vitro. In one embodiment, the invention further involves administration of a second agent (e.g., an adjuvant). In another embodiment, the pharmaceutical composition is administered in multiple doses over an extended period of time (e.g., 1 month, two months, three months). In other embodiments, the method involves further administering an adjuvant, boost, or facilitating agent before, during, or after administration of the composition.

In a related aspect, the invention features a method of polynucleotide therapy in a subject (e.g., mammal, such as a human) involving identifying a gene product to be expressed; preparing a composition according to a previous aspect, where the virus is an adenovirus or adeno-associated virus that expresses a coding sequence that codes for the gene product; and administering the composition to a subject. In one embodiment, the coding sequence encodes a polypeptide (e.g., a therapeutic polypeptide). In another embodiment, the administration is oral or intra-nasal.

In a related aspect, the invention features a kit containing the immunogenic composition of a previous aspect and instructions for use.

In a related aspect, the invention features a kit containing the vaccine of a previous aspect and instructions for use.

In another aspect, the invention features a kit containing the virus of a previous aspect and instructions for use. In one embodiment, the kit is for use in treating a viral infection or for use in polynucleotide therapy.

In various embodiments of any previous aspect, the cell expresses an increased level of a siat7e, lama4, cdk13, cox15, egr1, or gas6 nucleic acid molecule or polypeptide relative to a control cell. In other embodiments, the cell expresses a decreased level of a siat7e, lama4, cdk13, cox15, egr1, or gas6 nucleic acid molecule or polypeptide relative to a control cell. In further embodiments, the cell expresses an increased level of siat7e nucleic acid molecule or polypeptide and a decreased level of lama4 nucleic acid molecule or polypeptide relative to a control cell.

In other embodiments, the mutation is a deletion, missense mutation, or frameshift. In still other embodiments of the above aspects, the virus is influenza virus (e.g., human, avian, and canine influenza virus), pneumovirus, hoof in mouth disease, or varicella zoster.

In another embodiment, the cell is a mammalian cell cultured in vitro, cultured in suspension (e.g., in a bioreactor). In other embodiments, the cell is a madin darby canine kidney (MDCK) or a Vero cell. In another embodiment, the cell has altered growth characteristics (e.g., increased or decreased adhesive characteristics, growth to increased cell density or an increased cell population size) relative to a control cell. In one embodiment, adhesive characteristics are measured by cell aggregation or in a shear flow chamber. In another embodiment, the cell expresses increased levels of an immunogenic composition relative to a control cell. In another embodiment, the cell expresses increased levels of a vaccine, virus, or recombinant polypeptide relative to a control cell. In other embodiments of an aspect of the invention delineated herein, the producing step further involves infecting cells with the virus (e.g., influenza virus, pneumovirus, hoof in mouth disease, adenovirus, adeno-associated virus, and varicella zoster) to produce an increased yield of virus relative to a control cell. The virus is an adenovirus.

In any one of the embodiments, the cdk13 nucleic acid molecule corresponds to SEQ ID NO: 1. In any one of the embodiments, the cdk13 polypeptide is encoded by the amino acid sequence corresponding to SEQ ID NO: 2.

In any one of the embodiments, the siat7e nucleic acid molecule corresponds to SEQ ID NO: 3. In any one of the embodiments, the siat7e polypeptide is encoded by the amino acid sequence corresponding to SEQ ID NO: 4.

In any one of the embodiments, the lama4 nucleic acid molecule corresponds to SEQ ID NO: 5. In any one of the embodiments, the lama4 polypeptide is encoded by the amino acid sequence corresponding to SEQ ID NO: 6.

In any one of the embodiments, the cox15 nucleic acid molecule corresponds to SEQ ID NO: 7. In any one of the embodiments, the cox15 polypeptide is encoded by the amino acid sequence corresponding to SEQ ID NO: 8.

In any one of the embodiments, the egr1 nucleic acid molecule corresponds to SEQ ID NO: 9. In any one of the embodiments, the egr1 polypeptide is encoded by the amino acid sequence corresponding to SEQ ID NO: 10.

In any one of the embodiments, the gash nucleic acid molecule corresponds to SEQ ID NO: 11. In any one of the embodiments, the gas6 polypeptide is encoded by the amino acid sequence corresponding to SEQ ID NO: 12.

In any one of the embodiments, the gap43 nucleic acid molecule corresponds to SEQ ID NO: 13. In any one of the embodiments, the gap43 polypeptide is encoded by the amino acid sequence corresponding to SEQ ID NO: 14.

In any one of the embodiments, the map3k9 nucleic acid molecule corresponds to SEQ ID NO: 15. In any one of the embodiments, the map3k9 polypeptide is encoded by the amino acid sequence corresponding to SEQ ID NO: 16.

Other features and advantages of the invention will be apparent from the detailed description, and from the claims.

### DEFINITIONS

By “agent” is meant any small molecule chemical compound, antibody, nucleic acid molecule, or polypeptide, or fragments thereof.

By “alteration” is meant a change (increase or decrease) in the expression levels of a gene or polypeptide as detected by standard art known methods such as those described above. As used herein, an alteration includes a 10% change in expression levels, preferably a 25% change, more preferably a 40% change, and more preferably a 50%, 75%, 85%, 100% or greater change in expression levels.

By “anchorage-dependent cell” is meant a cell that requires interaction with a substrate for its survival, growth, or proliferation.

By “anchorage-independent cell” is meant a cell that does not require interaction with a substrate for its survival, growth, or proliferation.

By “cell growth characteristics” is meant the properties that define the growth of an unaltered reference cell. Such properties include cell aggregation, rate of cell proliferation, cell adhesion, or cell mortality.

By “cellular adhesion” is meant a cell-cell interaction or a cell-substrate interaction. Methods of measuring cell adhesion are known in the art and are described herein. In particular, such methods include measuring cell aggregation or measuring a cell-substrate interaction in a shear flow chamber.

By “cox15 nucleic acid molecule” is meant a nucleic acid molecule that encodes a cox15 polypeptide. An exemplary cox15 polynucleotide is provided at GenBank Accession No.: NM_{—}078470.

By a “cox15 polypeptide” is meant a polypeptide having substantial identity to GenBank Accession No. NP_{—}510870 or a fragment thereof having cytochrome oxidase activity.

By a “cdk13 nucleic acid molecule” is meant a nucleic acid molecule that encodes a cdk13 polypeptide. An exemplary cdk13 nucleic acid molecule is provided at GenBank Accession No: NM016508.

By a “cdk13 polypeptide” is meant a polypeptide having substantial identity to GenBank Accession No. NP_{—}057592 or a fragment thereof having cdk13 kinase activity.

By “cellular mortality” is meant a cell not having the ability to continue to grow and divide indefinitely. Cells that continue to grow and divide indefinitely are “immortalized cells.”

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “differentially expressed” is meant an increase or decrease in the expression of a polynucleotide or polypeptide relative to a reference level of expression.

By “egr1 nucleic acid molecule” is meant a nucleic acid molecule encoding an egr1 polypeptide. An exemplary egr1 nucleic acid molecule is provided at GenBank Accession No. NM_{—}001964.

By “egr1 polypeptide” is meant a protein having substantial identity to GenBank Accession No. NP_{—}001955 or a fragment thereof. In preferred embodiments, the protein has early growth response activity.

By “gas6 nucleic acid molecule” is meant a polynucleotide encoding a gas6 polypeptide. An exemplary gas6 nucleic acid molecule is provided at GenBank Accession No. NM_{—}000820.

By “gas6 polypeptide” is meant a protein having substantial identity to GenBank Accession No. NP_{—}000811 or a fragment thereof, In preferred embodiments, the protein has growth arrest specific activity.

By “gap43 nucleic acid molecule” is meant a polynucleotide encoding a gap43 polypeptide. An exemplary gas6 nucleic acid molecule is provided at GenBank Accession No. NM_{—}001130064.

By “gap43 polypeptide” is meant a protein having substantial identity to GenBank Accession No. NP_{—}001123536 or a fragment thereof, In preferred embodiments, the protein has growth arrest specific activity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

Nucleic acid molecules useful in the methods of the invention include any nucleic acid molecule that encodes a polypeptide or inhibitory nucleic acid molecule of the invention or a fragment thereof (e.g., cdk13, siat7e, lama4, cox15, egr1, gas6, map3k9, and gap43). Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule.

By “isolated nucleic acid molecule” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule which is transcribed from a DNA molecule, as well as a recombinant DNA which is part of a hybrid gene encoding additional polypeptide sequence.

By “lama4 nucleic acid molecule” is meant a polynucleotide that encodes a laminin α4 polypeptide. An exemplary human lama4 nucleic acid molecule is provided by Homo sapiens laminin, alpha 4 (LAMA4), isoform 1 precursor, corresponding to GenBank Accession No. NM_{—}001105206 that encodes a Homo sapiens laminin, alpha 4 (LAMA4), isoform 1 precursor polypeptide corresponding to GenBank Accession No. NP_{—}001098676. Another exemplary human lama4 nucleic acid molecule is provided by Homo sapiens laminin, alpha 4 (LAMA4), isoform 2 precursor, corresponding to GenBank Accession No. NM 001105207.1 that encodes a Homo sapiens laminin, alpha 4 (LAMA4), isoform 2 precursor polypeptide corresponding to GenBank Accession No. NP_{—}001098677.1. Another exemplary human lama4 nucleic acid molecule is provided by Homo sapiens laminin, alpha 4 (LAMA4), isoform 3 precursor, corresponding to GenBank Accession No. NM 001105208.1 that encodes a Homo sapiens laminin, alpha 4 (LAMA4), isoform 3 precursor polypeptide corresponding to GenBank Accession No. NP_{—}001098678.1. Another exemplary human lama4 nucleic acid molecule is provided by Homo sapiens laminin, alpha 4 (LAMA4), isoform 3 precursor, corresponding to GenBank Accession No. NM_{—}001105209.1 that encodes a Homo sapiens laminin, alpha 4 (LAMA4), isoform 3 precursor polypeptide corresponding to GenBank Accession No. NP_{—}001098679.1. An exemplary mouse (Mus musculus) laminin, alpha 4 (Lama4), polypeptide is encoded by the amino acid sequence corresponding to Gen Bank Accession No. NM_{—}010681.

By “laminin α4 polypeptide” is meant a protein having substantial identity to the amino acid sequences corresponding to of GenBank Accession No. NP_NP_{—}001098676, or a fragment thereof having a biological activity associated with laminin α4. Exemplary biological activities include promoting cell adhesion to a substrate.

By “map3k9 nucleic acid molecule” is meant a polynucleotide encoding a mitogen-activated protein kinase kinase kinase 9 polypeptide, and preferably where the encoded protein has kinase activity. An exemplary map3k9 nucleic acid molecule is provided at GenBank Accession No. NM_{—}033141.

By “mapk39 polypeptide” is meant a protein having substantial identity to GenBank Accession No. NP_{—}149132 or a fragment thereof. Preferably, the map3k9 polypeptide has kinase activity.

By “modulates” is meant increases or decreases.

By “operably linked” is meant that a first polynucleotide is positioned adjacent to a second polynucleotide that directs transcription of the first polynucleotide when appropriate molecules (e.g., transcriptional activator proteins) are bound to the second polynucleotide.

By “promoter” is meant a polynucleotide sufficient to direct transcription. Exemplary promoters suitable for expressing a polynucleotide or polypeptide of the invention in a mammalian cell include, but are not limited to, the CMV, U6, and H1 promoters.

By “reference” is meant a standard or control condition.

By “ribozyme” is meant an RNA that has enzymatic activity, possessing site specificity and cleavage capability for a target RNA molecule. Ribozymes can be used to decrease expression of a polypeptide. Methods for using ribozymes to decrease polypeptide expression are described, for example, by Turner et al., (Adv. Exp. Med. Biol. 465:303-318, 2000) and Norris et al., (Adv. Exp. Med. Biol. 465:293-301, 2000).

By “sialyltransferase” is meant any enzyme that transfers sialic acid to an oligosaccharide. Sialyltransferases add sialic acid to the terminal portions of the sialylated glycolipids (gangliosides) or to the N- or O-linked sugar chains of glycoproteins. There are about twenty different sialyltransferases which can be distinguished on the basis of the acceptor structure on which they act and on the type of sugar linkage they form. Any sialyltransferase is suitable for use in the invention as claimed. In preferred embodiments, the sialyltransferase is siat7e.

By “siat7e (sialyltransferase 7E) nucleic acid molecule” is meant a polynucleotide that encodes a Homo sapiens ST6 (alpha-N-acetyl-neuraminyl-2,3-beta-galactosyl-1,3)-N-acetylgalactosaminide alpha-2,6-sialyltransferase 5 (ST6GALNAC5) polypeptide. An exemplary nucleic acid sequence corresponds to GenBank Accession No. NM_{—}030965. An exemplary homo sapiens siat7e polypeptide is encoded by the amino acid sequence corresponding to Gen Bank Accession No. NM_{—}030965.

By “siat7e polypeptide” is meant a protein having substantial identity to GenBank accession No. NP_{—}112227.1, or a fragment thereof having sialyltransferase activity.

By “substantially identical” is meant a polypeptide or nucleic acid molecule exhibiting at least 75% identity to a reference amino acid sequence (for example, any one of the amino acid sequences described herein) or nucleic acid sequence (for example, any one of the nucleic acid sequences described herein). Preferably, such a sequence is at least 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transgenic” is meant any cell which includes a DNA sequence which is inserted by artifice into a cell and becomes part of the genome of the organism which develops from that cell, or part of a heritable extra chromosomal array.

By “vaccine” is meant to refer to an immunogenic composition providing or aiding in prevention of disease. In certain embodiments, a vaccine is a composition that can provide or aid in a cure of a disease. In still other embodiments, a vaccine composition can provide or aid in amelioration of a disease. Further embodiments of a vaccine immunogenic composition can be used as therapeutic and/or prophylactic agents.

### BRIEF DESCRIPTION OF THE DRAWINGS

FIG. 1 (A-C) shows parental and siat7e-expressing MDCK cells grown in T flasks. Panel (A) shows Parental MDCK cells: Panel (B) shows Clone 1, isolated from the siat7e-expressing pool. Panel (C) shows Clone 2, isolated from the siat7e-expressing pool.

FIGS. 2 (A & B) shows mRNA expression of human siat7e and endogenous GAPDH in parental MDCK and in clones 1 and 2 of the siat7e-expressing cells. Panel (A) shows end-point RT-PCR. Panel (B) shows real-time PCR.

FIGS. 3 (A & B) shows FITC signal distribution obtained by FACS analysis of parental and siat7e-expressing MDCK cells with and without ferritin. Panel (A) shows MDCK cells without ferritin treatment. Panel (B) shows MDCK cells with ferritin treatment. Parental MDCK cells ( - - - ), siat7e-expressing cells ( [symbol] ).

FIG. 4 (A-D) shows growth parameters of parental MDCK cells (-∘-) and siat7e-expressing MDCK cells (-□-) in shake flask in suspension and in monolayer in T flasks. T-flasks are shown in panels (A)-(C). Panel (A) shows viable cell density (VCD). Panel (B) shows viability %. Panel (C) shows glucose consumption and lactate production (shaded) in g/L. Shake flasks are shown in panels (D)-(F). Panel (D) shows growth in viable cell density (VCD). Panel (E) shows viability %. Panel (F) shows glucose consumption and lactate production (shaded) in g/L.

FIG. 5 shows HA production (-□-) and cell viability following infection of siat7e-expressing MDCK cells with influenza B virus (--). Cell viability of siat7e-expressing MDCK cells without infection are also shown (-∘-).

FIGS. 6 (A & B) shows two graphs showing the performance of the siat7e-expressing MDCK cells in a WAVE bioreactor. FIG. 6a displays viable cell density as a function of time and FIG. 6b indicates the viability % at the corresponding times.

FIG. 7 shows the kinetics of HA production, measured by titration against chicken red blood cells, at different MOI and different maintenance media. In the graph, SC, serum containing media; SF, serum free media; CTL, control (no virus).

FIG. 8 shows the tumorigenicity analysis of the parental (T038) and the siat7e-expressing (T034) MDCK cells. The results are expressed in tumor producing dose at the 50% end point (TPD₅₀), i.e. the number of cells required for tumor formation, TPD₅₀ Log 10 over a period of 26 weeks. Results were generated from 5 nude mice at each dosage level.

### DETAILED DESCRIPTION OF THE INVENTION

The present invention is based, in part, on the finding that MDCK cells can be considered as an alternative to embryonated eggs for the influenza virus propagation and hemagglutinin (HA) production intended for vaccine manufacturing. Previously, MDCK cells were found suitable for virus production but their inability to grow in suspension burdens the process of scale up and production capability.

As described herein, the present invention features methods of producing immunogenic compositions and viruses, methods of treating and preventing viral infection, and methods of producing an immune response using cells that express a sialyltransferase or a laminin. In particular embodiments, the methods are directed to cells that express a polypeptide selected from the group consisting of cdk13, siat7e, lama4, cox15, egr1, gas6, map3k9, and gap43, and a virus.

The present invention also features methods of producing immunogenic compositions and viruses, methods of treating and preventing viral infection, and methods of producing an immune response where the cell comprises a mutation that alters the expression or activity of a sialyltransferase or a laminin. In particular embodiments, the methods are directed to cells that comprise a mutation that alters the express a polypeptide selected from the group consisting of cdk13, siat7e, lama4, cox15, egr1, gas6, map3k9, and gap43, and a virus.

The invention is based, at least in part, on the observations that cell adhesive characteristics and recombinant protein production can be altered by modulating the expression of genes (e.g., cdk13, siat7e, lama4, cox15, egr1, gas6, map3k9, and gap43) that are differentially expressed in anchorage-dependent and anchorage-independent cell lines. Specifically, recombinant polypeptide expression is increased in cells transfected with an expression vector that encodes cdk13, cox15, egr1 or gas6; and alterations in laminin α4, sialyltransferase 7E, cdk13, cox15, egr1 or gas6 modulate cellular adhesion.

The invention is based, in part, on the finding the when cell adhesive characteristics and recombinant protein production are altered by modulating gene expression, the cells can be grown to high density in suspension and are particularly useful for vaccine production, particularly vaccines for the treatment or prevention of a viral infection, such as viral influenza.

### Cellular Adhesion

An important cellular property in biotechnology applications is adherence, which refers to a cell's ability to attach to a surface and grow. Anchorage-independent cell lines are cell lines that grow without adhering to a surface, while anchorage-dependent cell lines must adhere to a surface to grow. Depending on the biotechnology application, anchorage-independent or anchorage-dependent cell lines may be preferred. Being able to manipulate the cellular feature of adhesion would, therefore, benefit biotechnology applications.

A variety of studies have been conducted to evaluate the importance of cellular properties for the production of specific products. Researchers have also identified possible pathways to modify cellular properties by employing specific selection methods. In relation to adhesion, most studies have focused on either quantifying observations relating to adhesion at a genetic level or exploring the effects of specific compounds on adhesion. For instance, selenite, a hydrous calcium sulfate, has been shown to reduce the ability of HeLa cells to attach to fibronectin. In another series of experiments, researchers showed that blocking the expression of pten, a tumor suppressor gene, in 293T cells using siRNA resulted in a loss of adhesion as well as a change in cell morphology (Mise-Omata et al., Biochem. Biophys. Res. Commun. 328, 1034-1042). Other studies have highlighted a number of genes thought to be involved in mediating adhesion such as rhoA, racl, and cdc42 (Mise-Omata et al., Biochem. Biophys. Res. Commun. 328, 1034-1042; Hatzimanikatis and Lee, Metab. Eng. 1, 275-281, 1999). The present invention employs bioinformatic methods to identify genes that are differentially expressed in anchorage-dependent vs. anchorage independent cells. In addition, the method provides methods for modulating the adhesive characteristics of cells.

The present invention further provides methods of treating or preventing infectious diseases and/or disorders or symptoms, including viral infections which comprise administering a therapeutically effective amount of a pharmaceutical composition (e.g., immunogenic composition) comprising a virus or fragment thereof to a subject (e.g., a mammal such as a human). Thus, one embodiment is a method of treating a subject suffering from or susceptible to a viral disease or disorder or symptom thereof. The method includes the step of administering to the mammal a therapeutic amount of an amount of an immunogenic composition herein sufficient to treat the disease or disorder or symptom thereof, under conditions such that the disease or disorder is treated.

The methods herein include administering to the subject (including a subject identified as in need of such treatment) an effective amount of a compound described herein, or a composition described herein to produce such effect. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which viral infections may be implicated.

### Polynucleotides and Polypeptides

The present invention features methods of producing immunogenic compositions and viruses, methods of treating and preventing viral infection, and methods of producing an immune response using cells that express a virus and a polypeptide or an inhibitory nucleic acid molecule selected from the group consisting of, but not limited to, cdk13, siat7e, lama4, cox15, egr1, gas6, map3k9, and gap43, and a virus.

By a “cdk13 nucleic acid molecule” is meant a nucleic acid molecule that encodes a cdk13 polypeptide. An exemplary cdk13 nucleic acid molecule is provided at GenBank Accession No: NM016508, and corresponds to SEQ ID NO: 1, shown below:

| SEQ ID NO: 1 | |
|---|---|
| 1 | ggaactacgc agagccagac cagcgggacc acagaatggg ctgaggcggc ggcggctgtt |
| 61 | tggataaagt caacagcggg acgtggggcg tgacgccgta gtaaaagccc agcttgaaaa |
| 121 | tggagatgta tgaaaccctt ggaaaagtgg gagagggaag ttacggaaca gtcatgaaat |
| 181 | gtaaacataa gaatactggg cagatagtgg ccattaagat attttatgag agaccagaac |
| 241 | aatctgtcaa caaaattgcg atgagagaaa taaagtttct aaagcaattt catcacgaaa |
| 301 | acctggtcaa tctgattgaa gtttttagac agaaaaagaa aattcatttg gtatttgaat |
| 361 | ttattgacca cacagtatta gatgagttac aacattattg tcatggacta gagagtaagc |
| 421 | gacttagaaa atacctcttc cagatccttc gagcaattga ctatcttcac agtaataata |
| 481 | tcattcatcg agatataaaa cctgagaata ttttagtatc ccagtcagga attactaagc |
| 541 | tctgtgattt tggttttgca cgaacactag cagctcctgg ggacatttat acggactatg |
| 601 | tggccacacg ctggtataga gctcccgaat tagtattaaa agatacttct tatggaaaac |
| 661 | ctgtggatat ctgggctttg ggctgtatga tcattgagat ggccactgga aatccctatc |
| 721 | ttcctagtag ttctgatttg gatttactcc ataaaattgt tttgaaagtg ggcaatttgt |
| 781 | cacctcactt gcagaatatc ttttccaaga gccccatttt tgctggggta gttcttcctc |
| 841 | aagttcaaca ccccaaaaat gcaagaaaaa aatatccaaa gcttaatgga ttgttggcag |
| 901 | atatagttca tgcttgttta caaattgatc ctgctgacag gatatcatct agtgatcttt |
| 961 | tgcatcatga gtattttact agagatggat ttattgaaaa attcatgcca gaactgaaag |
| 1021 | ctaaattact gcaggaagca aaagtcaatt cattaataaa gccaaaagag agttctaaag |
| 1081 | aaaatgaact caggaaagat gaaagaaaaa cagtttatac caatacactg ctaagtagtt |
| 1141 | cagttttggg aaaggaaata gaaaaagaga aaaagcccaa ggagatcaaa gtcagagtta |
| 1201 | ttaaagtcaa aggaggaaga ggagatatct cagaaccaaa aaagaaagag tatgaaggtg |
| 1261 | gacttggtca acaggatgca aatgaaaatg ttcatcctat gtctccagat acaaaacttg |
| 1321 | taaccattga accaccaaac cctatcaatc ccagcactaa ctgtaatggc ttgaaagaaa |
| 1381 | atccacattg cggaggttct gtgacaatgc cacccatcaa tctaactaac agtaatttga |
| 1441 | tggctgcaaa tctcagttca aatctctttc accccagtgt gaggtgagct gtaacagaga |
| 1501 | agaaacctaa ataatacaac attcctgtat aatggtattt caaagaatcg tgttcatagt |
| 1561 | gtctgtatgt aaactgaact tgaagaaaat atattgaaat taaagctgta taatgggcca |
| 1621 | aaaaaaaaaa aa |

By a “cdk13 polypeptide” is meant a polypeptide having substantial identity to GenBank Accession No. NP_{—}057592 or a fragment thereof having cdk13 kinase activity, and corresponds to SEQ ID NO: 2, shown below:

| SEQ ID NO: 2 | |
|---|---|
| 1 | memyetlgkv gegsygtvmk ckhkntgqiv aikifyerpe qsvnkiamre ikflkqfhhe |
| 61 | nlvnlievfr qkkkihlvfe fidhtvldel qhychglesk rlrkylfqil raidylhsnn |
| 121 | iihrdikpen ilvsqsgitk lcdfgfartl aapgdiytdy vatrwyrape lvlkdtsygk |
| 181 | pvdiwalgcm iiematgnpy lpsssdldll hkivlkvgnl sphlqnifsk spifagvvlp |
| 241 | qvqhpknark kypklnglla divhaclqid padrisssdl lhheyftrdg fiekfmpelk |
| 301 | akllqeakvn slikpkessk enelrkderk tvytntllss svlgkeieke kkpkeikvrv |
| 361 | ikvkggrgdi sepkkkeyeg glgqqdanen vhpmspdtkl vtieppnpin pstncnglke |
| 421 | nphcggsvtm ppinltnsnl maanlssnlf hpsvr |

By “siat7e nucleic acid molecule” is meant a polynucleotide that encodes a Homo sapiens ST6 (alpha-N-acetyl-neuraminyl-2,3-beta-galactosyl-1,3)-N-acetylgalactosaminide alpha-2,6-sialyltransferase 5 (ST6GALNAC5) polypeptide. An exemplary nucleic acid sequence corresponds to GenBank Accession No. NM_{—}030965. An exemplary homo sapiens siat7e polypeptide is encoded by the amino acid sequence corresponding to Gen Bank Accession No. NM_{—}030965, and corresponds to SEQ ID NO: 3, shown below:

| SEQ ID NO: 3 | |
|---|---|
| 1 | ctctgcaaca gccgcgcttc ccgggtcccg cggctcccgc gcgcgatctg ccgcggccgg |
| 61 | ctgctgggca aaaatcagag ccgcctccgc cccattaccc atcatggaaa ccctccagga |
| 121 | aaaagtggcc ccggacgcgc gagcctgagg attctgcaca aaagaggtgc ccaaaatgaa |
| 181 | gaccctgatg cgccatggtc tggcagtgtg tttagcgctc accaccatgt gcaccagctt |
| 241 | gttgctagtg tacagcagcc tcggcggcca gaaggagcgg cccccgcagc agcagcagca |
| 301 | gcagcagcaa cagcagcagc aggcgtcggc caccggcagc tcgcagccgg cggcggagag |
| 361 | cagcacccag cagcgccccg gggtccccgc gggaccgcgg ccactggacg gatacctcgg |
| 421 | agtggcggac cacaagcccc tgaaaatgca ctgcagggac tgtgccctgg tgaccagctc |
| 481 | agggcatctg ctgcacagtc ggcaaggctc ccagattgac cagacagagt gtgtcatccg |
| 541 | catgaatgac gcccccacac gcggctatgg gcgtgacgtg ggcaatcgca ccagcctgag |
| 601 | ggtcatcgcg cattccagca tccagaggat cctccgcaac cgccatgacc tgctcaacgt |
| 661 | gagccagggc accgtgttca tcttctgggg ccccagcagc tacatgcggc gggacggcaa |
| 721 | gggccaggtc tacaacaacc tgcatctcct gagccaggtg ctgccccggc tgaaggcctt |
| 781 | catgattact cgccacaaga tgctgcagtt tgatgagctc ttcaagcagg agactggcaa |
| 841 | agacaggaag atatccaaca cttggctcag cactggctgg tttacaatga caattgcact |
| 901 | ggagctctgt gacaggatca atgtttatgg catggtgccc ccagacttct gcagggatcc |
| 961 | caatcaccct tcagtacctt atcattatta tgaacctttt ggacctgatg aatgtacaat |
| 1021 | gtacctctcc catgagcgag gacgcaaggg cagtcatcac cgctttatca cagagaaacg |
| 1081 | agtctttaag aactgggcac ggacattcaa tattcacttt tttcaaccag actggaaacc |
| 1141 | agaatcactt gctataaatc atcctgagaa taaacctgtg ttctaaggaa tgagcatgcc |
| 1201 | agactgtaat cccaggtatt cactgcatca gacaccgaga cactgaactt cctgagccac |
| 1261 | cagacaggaa agggtagcag aaaacagctt cactcctcag gaagtaccat ggacagacgc |
| 1321 | ctaccagggg tgacaaagca gtgcagttgg attgtaagga aaaattccgg aattaatgca |
| 1381 | tcctaatgaa tgttgtcccc ttcaatggtg ttaccttagg agctgaacat tcaattcagt |
| 1441 | tacaccacta tgactaaaaa cagtttggat ctcttagtat tgcctttgaa actgcaacat |
| 1501 | aagcaactca acaatattag ttgcattcct ttatagacat accatgtcaa agacgttttt |
| 1561 | ctatcaagtt gtattctttc ctgttctata acctttgtca tctgttagac tctgtatgtg |
| 1621 | tgatttgtaa aaagcaggct gaaactatgg acatgatttc tgaagagcac atctccactg |
| 1681 | actttcataa agcaaatgtc caatatttat ttattgagag ttttttagtg caatctgggc |
| 1741 | cagtattttt atagattatg attatgtggt aatttatcct tcctaactct ttaatcctga |
| 1801 | atgatggttg gaaatggcct agaattaggt tactctgttc acaatgctca ttgttagcat |
| 1861 | gcaattggta tttgacttgg aagtgttgtg ttgtattttt tgaaccccta ggcttcagga |
| 1921 | aaactgctct tttgtaaaaa gaatagcgat gacattttct aatgtgcaga aatgttccaa |
| 1981 | aaggacaaaa ttgaaaacca aaaactatgt tattaaaaca aaaaaatgct aaaaaaaaaa |
| 2041 | aaaaaaaa |

By “sialyltransferase 7E polypeptide” is meant a protein having substantial identity to GenBank accession No. NP_{—}112227.1, or a fragment thereof having sialyltransferase activity, and corresponds to SEQ ID NO: 4, shown below:

| SEQ ID NO: 4 | |
|---|---|
| 1 | mktlmrhgla vclalttmct slllvysslg gqkerppqqq qqqqqqqqqa satgssqpaa |
| 61 | esstqqrpgv pagprpldgy lgvadhkplk mhcrdcalvt ssghllhsrq gsqidqtecv |
| 121 | irmndaptrg ygrdvgnrts lrviahssiq rilrnrhdll nvsqgtvfif wgpssymrrd |
| 181 | gkgqvynnlh llsqvlprlk afmitrhkml qfdelfkqet gkdrkisntw lstgwftmti |
| 241 | alelcdrinv ygmvppdfcr dpnhpsvpyh yyepfgpdec tmylshergr kgshhrfite |
| 301 | krvfknwart fnihffqpdw kpeslainhp enkpvf |

By “lama4 nucleic acid molecule” is meant a polynucleotide that encodes a laminin α4 polypeptide. An exemplary human lama4 nucleic acid molecule is provided by Homo sapiens laminin, alpha 4 (LAMA4), isoform 1 precursor, corresponding to GenBank Accession No. NM_{—}001105206, and corresponds to SEQ ID NO: 5 shows below:

| SEQ ID NO: 5 | |
|---|---|
| 1 | agcttagagt gggagggcct gggagtagaa ggtaaaaagg gagtggtgag aatgaatgtg |
| 61 | agaaggaagc caggacagcg cagtccccag tcccgaacgg ccagggagag gaggtggcct |
| 121 | agcgctggcg gggctcaccc caatccgtct gccttttgat gccgtactct gctggttgcg |
| 181 | cagccacctc gggatactgc acacggagag gagggaaaat aagcgaggca ccgccgcacc |
| 241 | acgcgggaga cctacggaga cccacagcgc ccgagccctg gaagagcact actggatgtc |
| 301 | agcggagaaa tggctttgag ctcagcctgg cgctcggttc tgcctctgtg gctcctctgg |
| 361 | agcgctgcct gctcccgcgc cgcgtccggg gacgacaacg cttttccttt tgacattgaa |
| 421 | gggagctcag cggttggcag gcaagacccg cctgagacga gcgaaccccg cgtggctctg |
| 481 | ggacgcctgc cgcctgcggc cgagaaatgc aatgctggat tctttcacac cctgtcggga |
| 541 | gaatgtgtgc cctgcgactg taatggcaat tccaacgagt gtttggacgg ctcaggatac |
| 601 | tgtgtgcact gccagcggaa cacaacagga gagcactgtg aaaagtgtct ggatggttat |
| 661 | atcggagatt ccatcagggg agcaccccaa ttctgccagc cgtgcccctg tcccctgccc |
| 721 | cacttggcca attttgcaga atcctgctat aggaaaaatg gagctgttcg gtgcatttgt |
| 781 | aacgaaaatt atgctggacc taactgtgaa agatgtgctc ccggttacta tggaaacccc |
| 841 | ttactcattg gaagcacctg taagaaatgt gactgcagtg gaaattcaga tcccaacctg |
| 901 | atctttgaag attgtgatga agtcactggc cagtgtagga attgcttacg caacaccacc |
| 961 | ggattcaagt gtgaacgttg cgctcctggc tactatgggg acgccaggat agccaagaac |
| 1021 | tgtgcagtgt gcaactgcgg gggaggccca tgtgacagtg taaccggaga atgcttggaa |
| 1081 | gaaggttttg aaccccctac aggcatggac tgcccaacca taagctgtga taagtgcgtc |
| 1141 | tgggacctga ctgatgcact gcggttagca gcgctctcca tcgaggaagg caaatccggg |
| 1201 | gtgctgagcg tatcctctgg ggccgccgct cataggcacg tgaatgaaat caacgccacc |
| 1261 | atctacctcc tcaaaacaaa attgtcagaa agagaaaacc aatacgccct aagaaagata |
| 1321 | caaatcaaca atgctgagaa cacgatgaaa agccttctgt ctgacgtaga ggaattagtt |
| 1381 | gaaaaggaaa atcaagcctc cagaaaagga caacttgttc agaaggaaag catggacacc |
| 1441 | attaaccacg caagtcagct ggtagagcaa gcccatgata tgagggataa aatccaagag |
| 1501 | atcaacaaca agatgctcta ttatggggaa gagcatgaac ttagccccaa ggaaatctct |
| 1561 | gagaagctgg tgttggccca gaagatgctt gaagagatta gaagccgtca accatttttc |
| 1621 | acccaacggg agctcgtgga tgaggaggca gatgaggctt acgaactact gagccaggct |
| 1681 | gagagctggc agcggctgca caatgagacc cgcactctgt ttcctgtcgt cctggagcag |
| 1741 | ctggatgact acaatgctaa gttgtcagat ctccaggaag cacttgacca ggcccttaac |
| 1801 | tatgtcaggg atgccgaaga catgaacagg gccacagcag ccaggcagcg ggaccatgag |
| 1861 | aaacaacagg aaagagtgag ggaacaaatg gaagtggtga acatgtctct gagcacatct |
| 1921 | gcggactctc tgacaacacc tcgtctaact ctttcagaac ttgatgatat aataaagaat |
| 1981 | gcgtcaggga tttatgcaga aatagatgga gccaaaagtg aactacaagt aaaactatct |
| 2041 | aacctaagta acctcagcca tgatttagtc caagaagcta ttgaccatgc acaggacctt |
| 2101 | caacaagaag ctaatgaatt gagcaggaag ttgcacagtt cagatatgaa cgggctggta |
| 2161 | cagaaggctt tggatgcatc aaatgtctat gaaaatattg ttaattatgt tagtgaagcc |
| 2221 | aatgaaacag cagaatttgc tttgaacacc actgaccgaa tttatgatgc ggtgagtggg |
| 2281 | attgatactc aaatcattta ccataaagat gaaagtgaga acctcctcaa tcaagccaga |
| 2341 | gaactgcaag caaaggcaga gtctagcagt gatgaagcag tggctgacac tagcaggcgt |
| 2401 | gtgggtggag ccctagcaag gaaaagtgcc cttaaaacca gactcagtga tgccgttaag |
| 2461 | caactacaag cagcagagag aggggatgcc cagcagcgcc tggggcagtc tagactgatc |
| 2521 | accgaggaag ccaacaggac gacgatggag gtgcagcagg ccactgcccc catggccaac |
| 2581 | aatctaacca actggtcaca gaatcttcaa cattttgact cttctgctta caacactgca |
| 2641 | gtgaactctg ctagggatgc agtaagaaat ctgaccgagg ttgtccctca gctcctggat |
| 2701 | cagcttcgta cggttgagca gaagcgacct gcaagcaacg tttctgccag catccagagg |
| 2761 | atccgagagc tcattgctca gaccagaagt gttgccagca agatccaagt ctccatgatg |
| 2821 | tttgatggcc agtcagctgt ggaagtgcac tcgagaacca gtatggatga cttaaaggcc |
| 2881 | ttcacgtctc tgagcctgta catgaaaccc cctgtgaagc ggccggaact gaccgagact |
| 2941 | gcagatcagt ttatcctgta cctcggaagc aaaaacgcca aaaaagagta tatgggtctt |
| 3001 | gcaatcaaaa atgataatct ggtatacgtc tataatttgg gaactaaaga tgtggagatt |
| 3061 | cccctggact ccaagcccgt cagttcctgg cctgcttact tcagcattgt caagattgaa |
| 3121 | agggtgggaa aacatggaaa ggtgttttta acagtcccga gtctaagtag cacagcagag |
| 3181 | gaaaagttca ttaaaaaggg ggaattttcg ggagatgact ctctgctgga cctggaccct |
| 3241 | gaggacacag tgttttatgt tggtggagtg ccttccaact tcaagctccc taccagctta |
| 3301 | aacctgcctg gctttgttgg ctgcctggaa ctggccactt tgaataatga tgtgatcagc |
| 3361 | ttgtacaact ttaagcacat ctataatatg gacccctcca catcagtgcc atgtgcccga |
| 3421 | gataagctgg ccttcactca gagtcgggct gccagttact tcttcgatgg ctccggttat |
| 3481 | gccgtggtga gagacatcac aaggagaggg aaatttggtc aggtgactcg ctttgacata |
| 3541 | gaagttcgaa caccagctga caacggcctt attctcctga tggtcaatgg aagtatgttt |
| 3601 | ttcagactgg aaatgcgcaa tggttaccta catgtgttct atgattttgg attcagcggt |
| 3661 | ggccctgtgc atcttgaaga tacgttaaag aaagctcaaa ttaatgatgc aaaataccat |
| 3721 | gagatctcaa tcatttacca caatgataag aaaatgatct tggtagttga cagaaggcat |
| 3781 | gtcaagagca tggataatga aaagatgaaa atacctttta cagatatata cattggagga |
| 3841 | gctcctccag aaatcttaca atccagggcc ctcagagcac accttcccct agatatcaac |
| 3901 | ttcagaggat gcatgaaggg cttccagttc caaaagaagg acttcaattt actggagcag |
| 3961 | acagaaaccc tgggagttgg ttatggatgc ccagaagact cacttatatc tcgcagagca |
| 4021 | tatttcaatg gacagagctt cattgcttca attcagaaaa tatctttctt tgatggcttt |
| 4081 | gaaggaggtt ttaatttccg aacattacaa ccaaatgggt tactattcta ttatgcttca |
| 4141 | gggtcagacg tgttctccat ctcactggat aatggtactg tcatcatgga tgtaaaggga |
| 4201 | atcaaagttc agtcagtaga taagcagtac aatgatgggc tgtcccactt cgtcattagc |
| 4261 | tctgtctcac ccacaagata tgaactgata gtagataaaa gcagagttgg gagtaagaat |
| 4321 | cctaccaaag ggaaaataga acagacacaa gcaagtgaaa agaagtttta cttcggtggc |
| 4381 | tcaccaatca gtgctcagta tgctaatttc actggctgca taagtaatgc ctactttacc |
| 4441 | agggtggata gagatgtgga ggttgaagat ttccaacggt atactgaaaa ggtccacact |
| 4501 | tctctttatg agtgtcccat tgagtcttca ccattgtttc tcctccataa aaaaggaaaa |
| 4561 | aatttatcca agcctaaagc aagtcagaat aaaaagggag ggaaaagtaa agatgcacct |
| 4621 | tcatgggatc ctgttgctct gaaactccca gagcggaata ctccaagaaa ctctcattgc |
| 4681 | cacctttcca acagccctag agcaatagag cacgcctatc aatatggagg aacagccaac |
| 4741 | agccgccaag agtttgaaca cttaaaagga gattttggtg ccaaatctca gttttccatt |
| 4801 | cgtctgagaa ctcgttcctc ccatggcatg atcttctatg tctcagatca agaagagaat |
| 4861 | gacttcatga ctctattttt ggcccatggc cgcttggttt acatgtttaa tgttggtcac |
| 4921 | aaaaaactga agattagaag ccaggagaaa tacaatgatg gcctgtggca tgatgtgata |
| 4981 | tttattcgag aaaggagcag tggccgactg gtaattgatg gtctccgagt cctagaagaa |
| 5041 | agtcttcctc ctactgaagc tacctggaaa atcaagggtc ccatttattt gggaggtgtg |
| 5101 | gctcctggaa aggctgtgaa aaatgttcag attaactcca tctacagttt tagtggctgt |
| 5161 | ctcagcaatc tccagctcaa tggggcctcc atcacctctg cttctcagac attcagtgtg |
| 5221 | accccttgct ttgaaggccc catggaaaca ggaacttact tttcaacaga aggaggatac |
| 5281 | gtggttctag atgaatcttt caatattgga ttgaagtttg aaattgcatt tgaagtccgt |
| 5341 | cccagaagca gttccggaac cctggtccac ggccacagtg tcaatgggga gtacctaaat |
| 5401 | gttcacatga aaaatggaca ggtcatagtg aaagtcaata atggcatcag agatttttcc |
| 5461 | acctcagtta cacccaagca gagtctctgt gatggcagat ggcacagaat tacagttatt |
| 5521 | agagattcta atgtggttca gttggatgtg gactctgaag tgaaccatgt ggttggaccc |
| 5581 | ctgaatccaa aaccaattga tcacagggag cctgtgtttg ttggaggtgt tccagaatct |
| 5641 | ctactgacac cacgcttggc ccccagcaaa cccttcacag gctgcatacg ccactttgtg |
| 5701 | attgatggac acccagtgag cttcagtaaa gcagccctgg tcagcggcgc cgtaagcatc |
| 5761 | aactcctgtc cagcagcctg acatgacaga gcacagctgc ccaaatacaa agttctttag |
| 5821 | agcactgaaa gaaacacaaa gccagccagg aggaacagta actcttcctt cgggtggaag |
| 5881 | ctttcatcga gttgaacagg acttaaacga atcatcaggg accggatatt tcttatttct |
| 5941 | catttggatt cttaaccttg aatccaaagt gtctgcaatg gacaacaatt gaaggagtgg |
| 6001 | caaacttact tgtattgaga gcacacgcaa ttcctactgg tgaaattact gtttctgttt |
| 6061 | ctaataaaat agaagggatt ccaaataaac acttgcacac atttttgaag tgcggctaga |
| 6121 | ttctcagatt cacctttctt ccagggaaga taactttcaa tctatataaa aatctctgtc |
| 6181 | ctaaaactac ctttctttat tttgaagaga cttactaact tacatataat ctaaattaga |
| 6241 | tgatagattt gtttttagcc cttttgtttg gtctatcagt ataagaagaa tattttaggt |
| 6301 | ttatagctga agttatcaag gtttaataaa gtaaatttct aacagaatac tagaaaaatg |
| 6361 | cagtataatt taattttttc taaataagaa acacaggaaa tcaactactt tttccccttc |
| 6421 | cttatctcct taaaagaaaa ataaaattgt acatgagagg aggcttctgt aggttattat |
| 6481 | taccattatt gtgtgttcta tgggaatcat tgaggatatc acagcaaaaa cagtaggaca |
| 6541 | aaatcataaa attcaattta agagtacaca agtcctttta ttaaaagttt gctcctagcc |
| 6601 | tgggcaacat aatgagatcc catctctgca aaaaaatttg tacatgggca tacacctgta |
| 6661 | gtcccagcta cttgggaggc tgagacggga ggatcgctta agctcaggag ttcaaggctg |
| 6721 | cagtgagcta tgactgctga ctgtacctgc actccagcct gggcaacaga gtgagatcct |
| 6781 | gtctcaaaaa caaagtgtgc tctccacata cctgcaacac aactagtctt atttctaaaa |
| 6841 | tgttataatc ttttttccaa gtagctacat taatatagtc tagaaaaaaa tggacttgaa |
| 6901 | tagctggtag aatattaaaa tatagaaatg aaataaaaga attatatcta aaaacctcaa |
| 6961 | ctcagaagac agaaaaagag aaaataggcc ctgatatcaa cagaattaac aatacataaa |
| 7021 | aggagtaact tttgagggga gaggatataa aatattttga ggaattacca aggggaataa |
| 7081 | aacaatgtta ccttgaaatg attatatata tattacatat tggtatatat gtccatacct |
| 7141 | acctatatcc cctgctaccc ttctgtctga aatatacaaa taatgataat gttgaagata |
| 7201 | tcgataaaca tagctaatgt ctgttcatag aggacttact aagtgccagc caccatgata |
| 7261 | agctaaagtt aattatttta tttgttc |

By “lama4 polypeptide” is meant a polypeptide having substantial identity to GenBank Accession No. NP_{—}001098676 or fragment thereof, and corresponds to SEQ ID NO: 6, shown below:

| SEQ ID NO: 6 | |
|---|---|
| 1 | malssawrsv lplwllwsaa csraasgddn afpfdiegss avgrqdppet seprvalgrl |
| 61 | ppaaekcnag ffhtlsgecv pcdcngnsne cldgsgycvh cqrnttgehc ekcldgyigd |
| 121 | sirgapqfcq pcpcplphla nfaescyrkn gavrcicnen yagpncerca pgyygnplli |
| 181 | gstckkcdcs gnsdpnlife dcdevtgqcr nclrnttgfk cercapgyyg dariakncav |
| 241 | cncgggpcds vtgecleegf epptgmdcpt iscdkcvwdl tdalrlaals ieegksgvls |
| 301 | vssgaaahrh vneinatiyl lktklseren qyalrkiqin naentmksll sdveelveke |
| 361 | nqasrkgqlv qkesmdtinh asqlveqahd mrdkiqeinn kmlyygeehe lspkeisekl |
| 421 | vlaqkmleei rsrqpfftqr elvdeeadea yellsqaesw qrlhnetrtl fpvvleqldd |
| 481 | ynaklsdlqe aldqalnyvr daedmnrata arqrdhekqq ervreqmevv nmslstsads |
| 541 | lttprltlse lddiiknasg iyaeidgaks elqvklsnls nlshdlvqea idhaqdlqqe |
| 601 | anelsrklhs sdmnglvqka ldasnvyeni vnyvseanet aefalnttdr iydaysgidt |
| 661 | qiiyhkdese nllnqarelq akaesssdea vadtsrrvgg alarksalkt rlsdavkqlq |
| 721 | aaergdaqqr lgqsrlitee anrttmevqq atapmannlt nwsqnlqhfd ssayntavns |
| 781 | ardavrnlte vvpqlldqlr tveqkrpasn vsasiqrire liaqtrsvas kiqvsmmfdg |
| 841 | qsavevhsrt smddlkafts lslymkppvk rpeltetadq filylgskna kkeymglaik |
| 901 | ndnlvyvynl gtkdveipld skpvsswpay fsivkiervg khgkvfltvp slsstaeekf |
| 961 | ikkgefsgdd slldldpedt vfyvggvpsn fklptslnlp gfvgclelat lnndvislyn |
| 1021 | fkhiynmdps tsvpcardkl aftqsraasy ffdgsgyavv rditrrgkfg qvtrfdievr |
| 1081 | tpadnglill mvngsmffrl emrngylhvf ydfgfsggpv hledtlkkaq indakyheis |
| 1141 | iiyhndkkmi lvvdrrhvks mdnekmkipf tdiyiggapp eilqsralra hlpldinfrg |
| 1201 | cmkgfqfqkk dfnlleqtet lgvgygcped slisrrayfn gqsfiasiqk isffdgfegg |
| 1261 | fnfrtlqpng llfyyasgsd vfsisldngt vimdvkgikv qsvdkqyndg lshfvissvs |
| 1321 | ptryelivdk srvgsknptk gkieqtqase kkfyfggspi saqyanftgc isnayftrvd |
| 1381 | rdvevedfqr ytekvhtsly ecpiessplf llhkkgknls kpkasqnkkg gkskdapswd |
| 1441 | pvalklpern tprnshchls nspraiehay qyggtansrq efehlkgdfg aksqfsirlr |
| 1501 | trsshgmify vsdqeendfm tlflahgrlv ymfnvghkkl kirsqekynd glwhdvifir |
| 1561 | erssgrlvid glrvleeslp pteatwkikg piylggvapg kavknvqins iysfsgclsn |
| 1621 | lqlngasits asqtfsvtpc fegpmetgty fsteggyvvl desfniglkf eiafevrprs |
| 1681 | ssgtlvhghs vngeylnvhm kngqvivkvn ngirdfstsv tpkqslcdgr whritvirds |
| 1741 | nvvqldvdse vnhvvgplnp kpidhrepvf vggvpesllt prlapskpft gcirhfvidg |
| 1801 | hpvsfskaal vsgavsinsc paa |

By “cox15 nucleic acid molecule” is meant a nucleic acid molecule that encodes a cox15 polypeptide. An exemplary cox15 polynucleotide is provided at GenBank Accession No.: NM_{—}078470 and corresponds to SEQ ID NO: 7 shown below.

| SEQ ID NO: 7 | |
|---|---|
| 1 | cacaaggtcg cagggccgtt atgaggggac cccgggactc gaaccttggc tccacagctg |
| 61 | agccattctc gctacctgcc cctcgtcacg ccctccgttt ccacaccttt aacgcctcaa |
| 121 | agatagaagg tgccgcccaa ggggctggaa ggagctgagg aaacgactcc agaagaaatc |
| 181 | accactgact acgactcccg ccggcccgcc ccggggagcc ttcggccgac cgtcccctcc |
| 241 | cccgccacct tccgcaccgg ccttcccgga cggtatccgc gctcgttttc gctcagagga |
| 301 | ggccccctgc cttttcatgc tccacgcgtt cctccctcgt gcgcctgcag tttccacttg |
| 361 | gaatttgggc tccggcgcgc accagctaag aagcgcgtca acagctgcgc gcgcccgtgc |
| 421 | gcgcgtcccc gacacctacg ccccagcagc ccccgcgaaa gcggagtcgc aacgcaggcg |
| 481 | cacttctgtt cgctccggtc cccagagaag gcggggctcc cgctgcccga cccggaagtg |
| 541 | cttctctttt ccttggcgga ggagggagac cacagagccc tgggttgtgg aagaggtggc |
| 601 | tgttccctgt catcagtatg cagcgattgc tctttccgcc gttgagggcc ttgaagggga |
| 661 | ggcagtatct gccgctcctg gctcctaggg cagcgcctag agcacagtgt gattgcatca |
| 721 | ggcgcccttt gaggccaggg caatacagca ccatctctga agtagctttg caatctggaa |
| 781 | ggggtacagt gtcccttccc tcaaaggctg ctgagcgggt ggtgggccga tggctcctgg |
| 841 | tctgcagtgg aacagtggct ggagcagtta ttcttggtgg agtaactagg ttgacagagt |
| 901 | ctggcctctc gatggtagat tggcatttaa taaaggagat gaagccacct acaagccaag |
| 961 | aggaatggga agcagaattc caaagatacc agcaatttcc agaatttaaa atcttgaatc |
| 1021 | atgatatgac actgacagaa ttcaagttca tctggtacat ggagtactca caccgaatgt |
| 1081 | ggggtcgcct tgtaggcctt gtgtacatcc tgcctgctgc ctacttttgg agaaagggct |
| 1141 | ggctcagccg tggcatgaaa ggacgtgttc ttgccctctg tggcctcgtc tgcttccagg |
| 1201 | gtctgttggg atggtatatg gtgaaaagtg gactagaaga aaaatcagac tcccatgaca |
| 1261 | tccctcgggt cagtcagtac cgccttgctg cccacctggg atcagccctg gttctttatt |
| 1321 | gtgccagctt gtggacctca ctgtcactgc tactccctcc gcacaagttg cctgaaaccc |
| 1381 | accaactcct acagttgaga cgatttgctc atggaacagc aggtctggtg ttccttacgg |
| 1441 | ccctctcagg ggcttttgtg gcagggctag atgctgggct tgtttataac tcctttccca |
| 1501 | aaatgggaga atcctggatc ccggaggacc tctttacctt ctcccccatc ctgaggaatg |
| 1561 | tttttgagaa tcccaccatg gtgcagtttg atcaccggat tctgggaatc acttcagtca |
| 1621 | ctgccattac agtgctctac ttcctctctc ggagaattcc ccttcctaga aggaccaaga |
| 1681 | tggcagcagt gactctgctg gctttggcgt atacacaggt gggcttgggc atcagcacgc |
| 1741 | tgctgatgta tgtcccaact cctctggccg ccactcacca gtcaggctcc ttggctttgc |
| 1801 | tcactggtgc tctttggctg atgaatgaac tccgaagagt cccaaaatga ttcttagagg |
| 1861 | accagcctcc tgggactgtg actgcttttg agagctcttc agagatcata agaacttggg |
| 1921 | cttttctacg agatgacctt gacataccaa gtggtttcca aatggtcaac ttacttaaaa |
| 1981 | atcttttcct gttttgagat agtcactgga tcaagaatgc attaagtgtg gttaccctaa |
| 2041 | atgttccctt ttaaatctgc ttttcatgtt gaaaatcagt tttaatgtag agaaagaaat |
| 2101 | gtctgccatt tgctgcttaa caggctttgt gtcaggtttt tcagtgttgg caagctcttg |
| 2161 | gttctacgtg gatgatttct acgtggatgt tctcctgagt ccttaattct gcctaaatag |
| 2221 | aatttcttta tgatcttaac ttcacttcca ttaggtgaag attgaaagga taggattgac |
| 2281 | atacccaaag tagccagctg gtcttcagca aaaaaaaaaa agctaatgtc ttctaatatc |
| 2341 | ctgattttca gaaatgggga aattgcagat tttgacaagc ttaatgtgta tatgtagcaa |
| 2401 | aatggttttt aagtacttgg aaaaggaggt aatcgccaaa tagttccatt tttttttttt |
| 2461 | tttttgagac agagtttcac tcttgttgcc caggctggag tacaatggcg tgatctcagc |
| 2521 | tcactctgca acctccactt cccaggttca agcgattctc atgcctcagc ctcccgagta |
| 2581 | gctgggacta caggcacaag tcaccatgcc tggctaattt tgtattttta gtagagaagg |
| 2641 | ggtttcacca cattggccgg gctggtcttg aactcctgac ctcaggtaat ccgcccatct |
| 2701 | cggcttccca aagtgctgag attacaggca tgggccacca cgcccagcta gttcctcttt |
| 2761 | tgataaggct gttaacatta caaggtcaga gagaggaatg gaatcgacag tataaattgg |
| 2821 | ttcctgagag ggtttcctaa cagctttgta ataagaaaaa tatggccttg atggtgtagt |
| 2881 | gcatggacat ctacccagat aaacaaattg ttgactgctg aattaatgtg attttggtct |
| 2941 | ctattgatat ttcatagggc cctgtcttat tcaactttac ttttaaaatc agtgatttgg |
| 3001 | atgaaggcat cagaaacatc tgatttgagg atggagcaac ccaatattgc taatataata |
| 3061 | gatgacagtg gcaactgaaa acagtcccag aatgacccca tcgagatgaa attaaacaga |
| 3121 | ttagaaatgt aacatactca tttaaattaa ataagttgta taagtttggg ctgaagaaac |
| 3181 | ctggccttat atcatttcat ataaaaaaga cgggatttta gttgtcctca ggttcaatat |
| 3241 | gaaccaagta gtattcatgt tgttattata tgataacaga aaaattagtg gtacttcagg |
| 3301 | ttttatcagt agaagtgtca cagccatagt caaaataccc ctaacatact gcatttcact |
| 3361 | ctgggtacca aatttaagca agatattgat ggccactaag tgtgtatcca gaagaagaga |
| 3421 | tcagaatgat gagtccagaa cccctgtcac acagggaata ggtgaaggaa ctagggatat |
| 3481 | ttaactggag cagagaacat ctggagagag agcaagattc ctgtccttga gaagttgaag |
| 3541 | ccgtctcatg cagaagaggg aatgagcttc cattatgctg cagagctggc agcaatggat |
| 3601 | gaaagttgca ggaagttagt tttagttctg tagaaggcaa gagtagggta gaccaaatca |
| 3661 | aatgctcact ggggtcaggc tgatggagac agtaacactg gcgagcaggt gccccagctc |
| 3721 | ggactatact gctgagttct ggtctgttgc tgccactcta caggtccagg gttgttagat |
| 3781 | cttctgggtt tttgttttgc tttttgaaat aaggaaaata agcttatctt aagtttattt |
| 3841 | ttccttggat atacctagat tccatacagt gacaatacaa atacaaagga agaacatctg |
| 3901 | gatttcatcc ttgacctcat ttacaaagca aaaagagatg ctggattcag atataaatgt |
| 3961 | ttaattttgc agtgtttaag tcagcaaatc ttctgttttt taaaaataag ccacatatct |
| 4021 | agatttttct gtgaaagctc tcaattaagt gttgggaact aatttcaaga ttttaaaaaa |
| 4081 | tgttatgcag gcttaacgtg tctgagagcc ataaatgacc taacgtttcc cgttagtctc |
| 4141 | tgaactaggt gatctcagtt ctctttcaac tccagtactc tgtaagtttc tgtgacctgt |
| 4201 | agtgtaccat tctcaggtgg tgatatggtt tggatgtttt gtcccctcca aatctcatgt |
| 4261 | tcaaagtgtg acattcagtg ttagaggtgg gcctagtagg aggtatttgg gtcatggggg |
| 4321 | cggatccctt atgtatgact ttgtgccatc cccatagtaa tgagtgagtt ctcactctgg |
| 4381 | ttgtttatgc aagagctggt tgtttaaaag agcctggaac ttcctcctct ctcgcttgct |
| 4441 | ctctctcacc atgtgacaca ctggctcccc ttcaccttct gccatgattg taagcttcgt |
| 4501 | gaggccctca ccagaagcag atgccagcgc catgcttcct atacagttct atgcagaaca |
| 4561 | gtgagccaaa taaacctctt ttctttctaa aaattgtcag tatttcctta tagcaatgca |
| 4621 | agccgactaa cacaggtgat tcttagcaaa acagtttgtc aatttttcat aaatgctgga |
| 4681 | ccctggctgg gcttatgcta ttgcctagaa gagattccca acttctctct tatttgaatc |
| 4741 | ttagaaaaat cccaaagccc aagcctcatc taagaccaat taagtcacaa tccctggagg |
| 4801 | aagtaaaagg catattttta aagttcccca ggtgattcca atgtgcagac aagtctaggg |
| 4861 | |

By “cox15 polypeptide” is meant a polypeptide having substantial identity to GenBank Accession No. NP_{—}510870 or a fragment there, and corresponds to SEQ ID NO: 8, shown below.

| SEQ ID NO: 8 | |
|---|---|
| 1 | mqrllfpplr alkgrqylpl lapraapraq cdcirrplrp gqystiseva lqsgrgtvsl |
| 61 | pskaaervvg rwllvcsgtv agavilggvt rltesglsmv dwhlikemkp ptsqeeweae |
| 121 | fqryqqfpef kilnhdmtlt efkfiwymey shrmwgrlvg lvyilpaayf wrkgwlsrgm |
| 181 | kgrvlalcgl vcfqgllgwy mvksgleeks dshdiprvsq yrlaahlgsa lvlycaslwt |
| 241 | slslllpphk lpethqllql rrfahgtagl vfltalsgaf vagldaglvy nsfpkmgesw |
| 301 | ipedlftfsp ilrnvfenpt mvqfdhrilg itsvtaitvl yflsrriplp rrtkmaavtl |
| 361 | lalaytqvgl gistllmyvp tplaathqsg slalltgalw lmnelrrvpk |

By “egr1 nucleic acid molecule” is meant a nucleic acid molecule encoding an egr1 polypeptide. An exemplary egr1 nucleic acid molecule is provided at GenBank Accession No. NM_{—}001964, and corresponds to SEQ ID NO: 9, shown below.

| SEQ ID NO; 9 | |
|---|---|
| 1 | gcgcagaact tggggagccg ccgccgccat ccgccgccgc agccagcttc cgccgccgca |
| 61 | ggaccggccc ctgccccagc ctccgcagcc gcggcgcgtc cacgcccgcc cgcgcccagg |
| 121 | gcgagtcggg gtcgccgcct gcacgcttct cagtgttccc cgcgccccgc atgtaacccg |
| 181 | gccaggcccc cgcaactgtg tcccctgcag ctccagcccc gggctgcacc cccccgcccc |
| 241 | gacaccagct ctccagcctg ctcgtccagg atggccgcgg ccaaggccga gatgcagctg |
| 301 | atgtccccgc tgcagatctc tgacccgttc ggatcctttc ctcactcgcc caccatggac |
| 361 | aactacccta agctggagga gatgatgctg ctgagcaacg gggctcccca gttcctcggc |
| 421 | gccgccgggg ccccagaggg cagcggcagc aacagcagca gcagcagcag cgggggcggt |
| 481 | ggaggcggcg ggggcggcag caacagcagc agcagcagca gcaccttcaa ccctcaggcg |
| 541 | gacacgggcg agcagcccta cgagcacctg accgcagagt cttttcctga catctctctg |
| 601 | aacaacgaga aggtgctggt ggagaccagt taccccagcc aaaccactcg actgcccccc |
| 661 | atcacctata ctggccgctt ttccctggag cctgcaccca acagtggcaa caccttgtgg |
| 721 | cccgagcccc tcttcagctt ggtcagtggc ctagtgagca tgaccaaccc accggcctcc |
| 781 | tcgtcctcag caccatctcc agcggcctcc tccgcctccg cctcccagag cccacccctg |
| 841 | agctgcgcag tgccatccaa cgacagcagt cccatttact cagcggcacc caccttcccc |
| 901 | acgccgaaca ctgacatttt ccctgagcca caaagccagg ccttcccggg ctcggcaggg |
| 961 | acagcgctcc agtacccgcc tcctgcctac cctgccgcca agggtggctt ccaggttccc |
| 1021 | atgatccccg actacctgtt tccacagcag cagggggatc tgggcctggg caccccagac |
| 1081 | cagaagccct tccagggcct ggagagccgc acccagcagc cttcgctaac ccctctgtct |
| 1141 | actattaagg cctttgccac tcagtcgggc tcccaggacc tgaaggccct caataccagc |
| 1201 | taccagtccc agctcatcaa acccagccgc atgcgcaagt accccaaccg gcccagcaag |
| 1261 | acgccccccc acgaacgccc ttacgcttgc ccagtggagt cctgtgatcg ccgcttctcc |
| 1321 | cgctccgacg agctcacccg ccacatccgc atccacacag gccagaagcc cttccagtgc |
| 1381 | cgcatctgca tgcgcaactt cagccgcagc gaccacctca ccacccacat ccgcacccac |
| 1441 | acaggcgaaa agcccttcgc ctgcgacatc tgtggaagaa agtttgccag gagcgatgaa |
| 1501 | cgcaagaggc ataccaagat ccacttgcgg cagaaggaca agaaagcaga caaaagtgtt |
| 1561 | gtggcctctt cggccacctc ctctctctct tcctacccgt ccccggttgc tacctcttac |
| 1621 | ccgtccccgg ttactacctc ttatccatcc ccggccacca cctcataccc atcccctgtg |
| 1681 | cccacctcct tctcctctcc cggctcctcg acctacccat cccctgtgca cagtggcttc |
| 1741 | ccctccccgt cggtggccac cacgtactcc tctgttcccc ctgctttccc ggcccaggtc |
| 1801 | agcagcttcc cttcctcagc tgtcaccaac tccttcagcg cctccacagg gctttcggac |
| 1861 | atgacagcaa ccttttctcc caggacaatt gaaatttgct aaagggaaag gggaaagaaa |
| 1921 | gggaaaaggg agaaaaagaa acacaagaga cttaaaggac aggaggagga gatggccata |
| 1981 | ggagaggagg gttcctctta ggtcagatgg aggttctcag agccaagtcc tccctctcta |
| 2041 | ctggagtgga aggtctattg gccaacaatc ctttctgccc acttcccctt ccccaattac |
| 2101 | tattcccttt gacttcagct gcctgaaaca gccatgtcca agttcttcac ctctatccaa |
| 2161 | agaacttgat ttgcatggat tttggataaa tcatttcagt atcatctcca tcatatgcct |
| 2221 | gaccccttgc tcccttcaat gctagaaaat cgagttggca aaatggggtt tgggcccctc |
| 2281 | agagccctgc cctgcaccct tgtacagtgt ctgtgccatg gatttcgttt ttcttggggt |
| 2341 | actcttgatg tgaagataat ttgcatattc tattgtatta tttggagtta ggtcctcact |
| 2401 | tgggggaaaa aaaaaaaaga aaagccaagc aaaccaatgg tgatcctcta ttttgtgatg |
| 2461 | atgctgtgac aataagtttg aacctttttt tttgaaacag cagtcccagt attctcagag |
| 2521 | catgtgtcag agtgttgttc cgttaacctt tttgtaaata ctgcttgacc gtactctcac |
| 2581 | atgtggcaaa atatggtttg gtttttcttt tttttttttt ttgaaagtgt tttttcttcg |
| 2641 | tccttttggt ttaaaaagtt tcacgtcttg gtgccttttg tgtgatgcgc cttgctgatg |
| 2701 | gcttgacatg tgcaattgtg agggacatgc tcacctctag ccttaagggg ggcagggagt |
| 2761 | gatgatttgg gggaggcttt gggagcaaaa taaggaagag ggctgagctg agcttcggtt |
| 2821 | ctccagaatg taagaaaaca aaatctaaaa caaaatctga actctcaaaa gtctattttt |
| 2881 | ttaactgaaa atgtaaattt ataaatatat tcaggagttg gaatgttgta gttacctact |
| 2941 | gagtaggcgg cgatttttgt atgttatgaa catgcagttc attattttgt ggttctattt |
| 3001 | tactttgtac ttgtgtttgc ttaaacaaag tgactgtttg gcttataaac acattgaatg |
| 3061 | cgctttattg cccatgggat atgtggtgta tatccttcca aaaaattaaa acgaaaataa |
| 3121 | agtagctgcg attggg |

By “egr1 polypeptide” is meant a protein having substantial identity to GenBank Accession No. NP_{—}001955 or a fragment thereof, and corresponds to SEQ ID NO: 10, shown below. In preferred embodiments, the protein has early growth response activity.

| SEQ ID NO: 10 | |
|---|---|
| 1 | maaakaemql msplqisdpf gsfphsptmd nypkleemml lsngapqflg aagapegsgs |
| 61 | nssssssggg ggggggsnss sssstfnpqa dtgeqpyehl taesfpdisl nnekvlvets |
| 121 | ypsqttrlpp itytgrfsle papnsgntlw peplfslvsg lvsmtnppas sssapspaas |
| 181 | sasasqsppl scavpsndss piysaaptfp tpntdifpep qsqafpgsag talqypppay |
| 241 | paakggfqvp mipdylfpqq qgdlglgtpd qkpfqglesr tqqpsltpls tikafatqsg |
| 301 | sqdlkalnts yqsqlikpsr mrkypnrpsk tppherpyac pvescdrrfs rsdeltrhir |
| 361 | ihtgqkpfqc ricmrnfsrs dhltthirth tgekpfacdi cgrkfarsde rkrhtkihlr |
| 421 | qkdkkadksv vassatssls sypspvatsy pspvttsyps pattsypspv ptsfsspgss |
| 481 | typspvhsgf pspsvattys svppafpaqv ssfpssavtn sfsastglsd mtatfsprti |
| 541 | eic |

By “gas6 nucleic acid molecule” is meant a polynucleotide encoding a gas6 polypeptide. An exemplary gas6 nucleic acid molecule is provided at GenBank Accession No. NM_{—}000820, and corresponds to SEQ ID NO: 11 shown below.

| SEQ ID NO: 11 | |
|---|---|
| 1 | ccgagcgctt gaggtgccgc agccgccgcc gccgccgccg ccgcgatgtg accttcaggg |
| 61 | ccgccaggac gggatgaccg gagcctccgc cccgcggcgc ccgcggctcg cctcggcctc |
| 121 | ccgggcgctc tgaccgcgcg tccccggccc gccatggccc cttcgctctc gcccgggccc |
| 181 | gccgccctgc gccgcgcgcc gcagctgctg ctgctgctgc tggccgcgga gtgcgcgctt |
| 241 | gccgcgctgt tgccggcgcg cgaggccacg cagttcctgc ggcccaggca gcgccgcgcc |
| 301 | tttcaggtct tcgaggaggc caagcagggc cacctggaga gggagtgcgt ggaggagctg |
| 361 | tgcagccgcg aggaggcgcg ggaggtgttc gagaacgacc ccgagacgga ttatttttac |
| 421 | ccaagatact tagactgcat caacaagtat gggtctccgt acaccaaaaa ctcaggcttc |
| 481 | gccacctgcg tgcaaaacct gcctgaccag tgcacgccca acccctgcga taggaagggg |
| 541 | acccaagcct gccaggacct catgggcaac ttcttctgcc tgtgtaaagc tggctggggg |
| 601 | ggccggctct gcgacaaaga tgtcaacgaa tgcagccagg agaacggggg ctgcctccag |
| 661 | atctgccaca acaagccggg tagcttccac tgttcctgcc acagcggctt cgagctctcc |
| 721 | tctgatggca ggacctgcca agacatagac gagtgcgcag actcggaggc ctgcggggag |
| 781 | gcgcgctgca agaacctgcc cggctcctac tcctgcctct gtgacgaggg ctttgcgtac |
| 841 | agctcccagg agaaggcttg ccgagatgtg gacgagtgtc tgcagggccg ctgtgagcag |
| 901 | gtctgcgtga actccccagg gagctacacc tgccactgtg acgggcgtgg gggcctcaag |
| 961 | ctgtcccagg acatggacac ctgtgaggac atcttgccgt gcgtgccctt cagcgtggcc |
| 1021 | aagagtgtga agtccttgta cctgggccgg atgttcagtg ggacccccgt gatccgactg |
| 1081 | cgcttcaaga ggctgcagcc caccaggctg gtagctgagt ttgacttccg gacctttgac |
| 1141 | cccgagggca tcctcctctt tgccggaggc caccaggaca gcacctggat cgtgctggcc |
| 1201 | ctgagagccg gccggctgga gctgcagctg cgctacaacg gtgtcggccg tgtcaccagc |
| 1261 | agcggcccgg tcatcaacca tggcatgtgg cagacaatct ctgttgagga gctggcgcgg |
| 1321 | aatctggtca tcaaggtcaa cagggatgct gtcatgaaaa tcgcggtggc cggggacttg |
| 1381 | ttccaaccgg agcgaggact gtatcatctg aacctgaccg tgggaggtat tcccttccat |
| 1441 | gagaaggacc tcgtgcagcc tataaaccct cgtctggatg gctgcatgag gagctggaac |
| 1501 | tggctgaacg gagaagacac caccatccag gaaacggtga aagtgaacac gaggatgcag |
| 1561 | tgcttctcgg tgacggagag aggctctttc taccccggga gcggcttcgc cttctacagc |
| 1621 | ctggactaca tgcggacccc tctggacgtc gggactgaat caacctggga agtagaagtc |
| 1681 | gtggctcaca tccgcccagc cgcagacaca ggcgtgctgt ttgcgctctg ggcccccgac |
| 1741 | ctccgtgccg tgcctctctc tgtggcactg gtagactatc actccacgaa gaaactcaag |
| 1801 | aagcagctgg tggtcctggc cgtggagcat acggccttgg ccctaatgga gatcaaggtc |
| 1861 | tgcgacggcc aagagcacgt ggtcaccgtc tcgctgaggg acggtgaggc caccctggag |
| 1921 | gtggacggca ccaggggcca gagcgaggtg agcgccgcgc agctgcagga gaggctggcc |
| 1981 | gtgctcgaga ggcacctgcg gagccccgtg ctcacctttg ctggcggcct gccagatgtg |
| 2041 | ccggtgactt cagcgccagt caccgcgttc taccgcggct gcatgacact ggaggtcaac |
| 2101 | cggaggctgc tggacctgga cgaggcggcg tacaagcaca gcgacatcac ggcccactcc |
| 2161 | tgcccccccg tggagcccgc cgcagcctag gcccccacgg gacgcggcag gcttctcagt |
| 2221 | ctctgtccga gacagccggg aggagcctgg gggctcctca ccacgtgggg ccatgctgag |
| 2281 | agctgggctt tcctctgtga ccatcccggc ctgtaacata tctgtaaata gtgagatgga |
| 2341 | cttggggcct ctgacgccgc gcactcagcc gtgggcccgg gcgcggggag gccggcgcag |
| 2401 | cgcagagcgg gctcgaagaa aataattctc tattattttt attaccaagc gcttctttct |
| 2461 | gactctaaaa tatggaaaat aaaatattta cagaaagctt tgtaaaaaaa aaaaaaaaaa |
| 2521 | a |

By “gas6 polypeptide” is meant a protein having substantial identity to GenBank Accession No. NP_{—}000811 or a fragment thereof, and corresponds to SEQ ID NO: 12, shown below. In preferred embodiments, the protein has growth arrest specific activity.

| SEQ ID NO: 12 | |
|---|---|
| 1 | mapslspgpa alrrapqlll lllaaecala allpareatq flrprqrraf qvfeeakqgh |
| 61 | lerecveelc sreearevfe ndpetdyfyp ryldcinkyg spytknsgfa tcvqnlpdqc |
| 121 | tpnpcdrkgt qacqdlmgnf fclckagwgg rlcdkdvnec sqenggclqi chnkpgsfhc |
| 181 | schsgfelss dgrtcqdide cadseacgea rcknlpgsys clcdegfays sqekacrdvd |
| 241 | eclqgrceqv cvnspgsytc hcdgrgglkl sqdmdtcedi lpcvpfsvak svkslylgrm |
| 301 | fsgtpvirlr fkrlqptrlv aefdfrtfdp egillfaggh qdstwivlal ragrlelqlr |
| 361 | yngvgrvtss gpvinhgmwq tisveelarn lvikvnrdav mkiavagdlf qperglyhln |
| 421 | ltvggipfhe kdlvqpinpr ldgcmrswnw lngedttiqe tvkvntrmqc fsvtergsfy |
| 481 | pgsgfafysl dymrtpldvg testwevevv ahirpaadtg vlfalwapdl ravplsvalv |
| 541 | dyhstkklkk qlvvlaveht alalmeikvc dgqehvvtvs lrdgeatlev dgtrgqsevs |
| 601 | aaqlqerlav lerhlrspvl tfagglpdvp vtsapvtafy rgcmtlevnr rlldldeaay |
| 661 | khsditahsc ppvepaaa |

By “gap43 nucleic acid molecule” is meant a polynucleotide encoding a gap43 polypeptide. An exemplary gash nucleic acid molecule is provided at GenBank Accession No. NM_{—}001130064, and corresponds to SEQ ID NO: 13.

| SEQ ID NO: 13 | |
|---|---|
| 1 | actgaaggct agagaacaat tccgagaaag agacggagag agagggaaga aaaagacaga |
| 61 | tagatagata ttggggggaa ggagaaaaaa ggagaagaga gggaagagag gacagcggag |
| 121 | agagagcacc agagagagag ggagagagag agagagcgct agagagaggg agcgagcatg |
| 181 | tgcgatgagc aatagctgtg gaccttacag ttgctgctaa ctgccctggt gtgtgtgagg |
| 241 | gagagagagg gagggaggga gagagagcgc gctagcgcga gagagcgagt gagcaagcga |
| 301 | gcagaaaaga ggtggagagg gggggaataa gaaagagaga gaaggaaagg agagaaggca |
| 361 | ggaagaaggc aagggacgag acaaccatgc tgtgctgtat gagaagaacc aaacagaatt |
| 421 | aaaagggaac ctggtctctg ggttgttttc aacatctcaa gtgtgaattt tccctgtcaa |
| 481 | aatcttcaca aggaaaatga gtcacagcat cacctgggtg acgaggtcat aacacctcag |
| 541 | cccttgctta aaaaatttta tttctacttt tctattgtaa agagatctca aaacaggaag |
| 601 | ataaaattgg actgacagct ctacagccta gtcttttaga cagtgaacta ggccagcatt |
| 661 | ggcagacact ggcgatgaca aagtcctgct ctgaattatg ccaccccgca ctccactttt |
| 721 | taccttgcct gggaggcttg aggaaaaatc ttcagagagc agttcgacct agtccttatt |
| 781 | cacttggctt cttgactttc tggatttcaa gggttgaaaa aaatgatgac gaccaaaaga |
| 841 | ttgaacaaga tggtatcaaa ccagaagata aagctcataa ggccgcaacc aaaattcagg |
| 901 | ctagcttccg tggacacata acaaggaaaa agctcaaagg agagaagaag gatgatgtcc |
| 961 | aagctgctga ggctgaagct aataagaagg atgaagcccc tgttgccgat ggggtggaga |
| 1021 | agaagggaga aggcaccact actgccgaag cagccccagc cactggctcc aagcctgatg |
| 1081 | agcccggcaa agcaggagaa actccttccg aggagaagaa gggggagggt gatgctgcca |
| 1141 | cagagcaggc agccccccag gctcctgcat cctcagagga gaaggccggc tcagctgaga |
| 1201 | cagaaagtgc cactaaagct tccactgata actcgccgtc ctccaaggct gaagatgccc |
| 1261 | cagccaagga ggagcctaaa caagccgatg tgcctgctgc tgtcactgct gctgctgcca |
| 1321 | ccacccctgc cgcagaggat gctgctgcca aggcaacagc ccagcctcca acggagactg |
| 1381 | gggagagcag ccaagctgaa gagaacatag aagctgtaga tgaaaccaaa cctaaggaaa |
| 1441 | gtgcccggca ggacgagggt aaagaagagg aacctgaggc tgaccaagaa catgcctgaa |
| 1501 | ctctaagaaa tggctttcca catccccacc ctcccctctc ctgagcctgt ctctccctac |
| 1561 | cctcttctca gctccactct gaagtccctt cctgtcctgc tcacgtctgt gagtctgtcc |
| 1621 | tttcccaccc actagccctc tttctctctg tgtggcaaac atttaaaaaa aaaaaaaaaa |
| 1681 | agcaggaaag atcccaagtc aaacagtgtg gcttaaacat tttttgtttc ttggtgttgt |
| 1741 | tatggcaagt ttttggtaat gatgattcaa tcattttggg aaattcttgc actgtatcca |
| 1801 | agttatttga tctggtgcgt gtggccctgt gggagtccac tttcctctct ctctctctct |
| 1861 | ctgttccaag tgtgtgtgca atgttccgtt catctgagga gtccaaaata tcgagtgaat |
| 1921 | tcaaaatcat ttttgttttc ctccttttca atgtgatgga atgaacaaaa aggaaaaaat |
| 1981 | tcaaaaaacc cagtttgttt taaaaataaa taaataaagc aaatgtgcca attagcgtaa |
| 2041 | acttgcggct ctaaggctcc tttttcaacc cgaatattaa taaatcatga gagtaatcaa |
| 2101 | ggtcaaaaaa aaaaaaaaaa aaaaaaaaaa aaaaaaaaaa aaa |

By “gap43 polypeptide” is meant a protein having substantial identity to GenBank Accession No. NP_{—}001123536 or a fragment thereof, and corresponds to SEQ ID NO: 14, shown below. In preferred embodiments, the protein has growth arrest specific activity.

| SEQ ID NO: 14 | |
|---|---|
| 1 | mtkscselch palhflpclg glrknlqrav rpspyslgfl tfwisrvekn dddqkieqdg |
| 61 | ikpedkahka atkiqasfrg hitrkklkge kkddvqaaea eankkdeapv adgvekkgeg |
| 121 | tttaeaapat gskpdepgka getpseekkg egdaateqaa pqapasseek agsaetesat |
| 181 | kastdnspss kaedapakee pkqadvpaav taaaattpaa edaaakataq pptetgessq |
| 241 | aeenieavde tkpkesarqd egkeeepead qeha |

By “map3k9 nucleic acid molecule” is meant a polynucleotide encoding a mitogen-activated protein kinase kinase kinase 9 polypeptide. An exemplary map3k9 nucleic acid molecule is provided at GenBank Accession No. NM_{—}033141, and corresponds to SEQ ID NO: 15, shown below.

| SEQ ID NO: 15 | |
|---|---|
| 1 | atggagccct ccagagcgct tctcggctgc ctagcgagcg ccgccgctgc cgccccgccg |
| 61 | ggggaggatg gagcaggggc cggggccgag gaggaggagg aggaggagga ggaggcggcg |
| 121 | gcggcggtgg gccccgggga gctgggctgc gacgcgccgc tgccctactg gacggccgtg |
| 181 | ttcgagtacg aggcggcggg cgaggacgag ctgaccctgc ggctgggcga cgtggtggag |
| 241 | gtgctgtcca aggactcgca ggtgtccggc gacgagggct ggtggaccgg gcagctgaac |
| 301 | cagcgggtgg gcatcttccc cagcaactac gtgaccccgc gcagcgcctt ctccagccgc |
| 361 | tgccagcccg gcggcgagga ccccagttgc tacccgccca ttcagttgtt agaaattgat |
| 421 | tttgcggagc tcaccttgga agagattatt ggcatcgggg gctttgggaa ggtctatcgt |
| 481 | gctttctgga taggggatga ggttgctgtg aaagcagctc gccacgaccc tgatgaggac |
| 541 | atcagccaga ccatagagaa tgttcgccaa gaggccaagc tcttcgccat gctgaagcac |
| 601 | cccaacatca ttgccctaag aggggtatgt ctgaaggagc ccaacctctg cttggtcatg |
| 661 | gagtttgctc gtggaggacc tttgaataga gtgttatctg ggaaaaggat tcccccagac |
| 721 | atcctggtga attgggctgt gcagattgcc agagggatga actacttaca tgatgaggca |
| 781 | attgttccca tcatccaccg cgaccttaag tccagcaaca tattgatcct ccagaaggtg |
| 841 | gagaatggag acctgagcaa caagattctg aagatcactg attttggcct ggctcgggaa |
| 901 | tggcaccgaa ccaccaagat gagtgcggca gggacgtatg cttggatggc acccgaagtc |
| 961 | atccgggcct ccatgttttc caaaggcagt gatgtgtgga gctatggggt gctactttgg |
| 1021 | gagttgctga ctggtgaggt gccctttcga ggcattgatg gcttagcagt cgcttatgga |
| 1081 | gtggccatga acaaactcgc ccttcctatt ccttctacgt gcccagaacc ttttgccaaa |
| 1141 | ctcatggaag actgctggaa tcctgatccc cactcacgac catctttcac gaatatcctg |
| 1201 | gaccagctaa ccaccataga ggagtctggt ttctttgaaa tgcccaagga ctccttccac |
| 1261 | tgcctgcagg acaactggaa acacgagatt caggagatgt ttgaccaact cagggccaaa |
| 1321 | gaaaaggaac ttcgcacctg ggaggaggag ctgacgcggg ctgcactgca gcagaagaac |
| 1381 | caggaggaac tgctgcggcg tcgggagcag gagctggccg agcgggagat tgacatcctg |
| 1441 | gaacgggagc tcaacatcat catccaccag ctgtgccagg agaagccccg ggtgaagaaa |
| 1501 | cgcaagggca agttcaggaa gagccggctg aagctcaagg atggcaaccg catcagcctc |
| 1561 | ccttctgatt tccagcacaa gttcacggtg caggcctccc ctaccatgga taaaaggaag |
| 1621 | agtcttatca acagccgctc cagtcctcct gcaagcccca ccatcattcc tcgccttcga |
| 1681 | gccatccagt tgacaccagg tgaaagcagc aaaacctggg gcaggagctc agtcgtccca |
| 1741 | aaggaggaag gggaggagga ggagaagagg gccccaaaga agaagggacg gacgtggggg |
| 1801 | ccagggacgc ttggtcagaa ggagcttgcc tcgggagatg aaggatcccc tcagagacgt |
| 1861 | gagaaagcta atggtttaag taccccatca gaatctccac atttccactt gggcctcaag |
| 1921 | tccctggtag atggatataa gcagtggtcg tccagtgccc ccaacctggt gaagggccca |
| 1981 | aggagtagcc cggccctgcc agggttcacc agccttatgg agatggcctt gctggcagcc |
| 2041 | agttgggtgg tgcccatcga cattgaagag gatgaggaca gtgaaggccc agggagtgga |
| 2101 | gagagtcgcc tacagcattc acccagccag tcctacctct gtatcccatt ccctcgtgga |
| 2161 | gaggatggcg atggcccctc cagtgatgga atccatgagg agcccacccc agtcaactcg |
| 2221 | gccacgagta cccctcagct gacgccaacc aacagcctca agcggggcgg tgcccaccac |
| 2281 | cgccgctgcg aggtggctct gctcggctgt ggggctgttc tggcagccac aggcctaggg |
| 2341 | tttgacttgc tggaagctgg caagtgccag ctgcttcccc tggaggagcc tgagccacca |
| 2401 | gcccgggagg agaagaaaag acgggagggt ctttttcaga ggtccagccg tcctcgtcgg |
| 2461 | agcaccagcc ccccatcccg aaagcttttc aagaaggagg agcccatgct gttgctagga |
| 2521 | gacccctctg cctccctgac gctgctctcc ctctcctcca tctccgagtg caactccaca |
| 2581 | cgctccctgc tgcgctccga cagcgatgaa attgtcgtgt atgagatgcc agtcagccca |
| 2641 | gtcgaggccc ctcccctgag tccatgtacc cacaaccccc tggtcaatgt ccgagtagag |
| 2701 | cgcttcaaac gagatcctaa ccaatctctg actcccaccc atgtcaccct caccaccccc |
| 2761 | tcgcagccca gcagtcaccg gcggactcct tctgatgggg cccttaagcc agagactctc |
| 2821 | ctagccagca ggagcccctc cagcaatggg ttgagcccca gtcctggagc aggaatgttg |
| 2881 | aaaaccccca gtcccagccg agacccaggt gaattccccc gtctccctga ccccaatgtg |
| 2941 | gtcttccccc caaccccaag gcgctggaac actcagcagg actctacctt ggagagaccc |
| 3001 | aagactctgg agtttctgcc tcggccgcgt ccttctgcca accggcaacg gctggaccct |
| 3061 | tggtggtttg tgtcccccag ccatgcccgc agcacctccc cagccaacag ctccagcaca |
| 3121 | gagacgccca gcaacctgga ctcctgcttt gctagcagta gcagcactgt agaggagcgg |
| 3181 | cctggacttc cagccctgct cccgttccag gcagggccgc tgcccccgac tgagcggacg |
| 3241 | ctcctggacc tggatgcaga ggggcagagt caggacagca ccgtgccgct gtgcagagcg |
| 3301 | gaactgaaca cacacaggcc tgccccttat gagatccagc aggagttctg gtcttagcac |
| 3361 | gaaaaggatt ggggcgggca agggggacag ccagcggaga tgaggggagc tggcgggcac |
| 3421 | agccctttct cagggttgga ccccctgaga tccagcccta cttcttgcac tgataatgca |
| 3481 | ctttgaagat ggaagggatg gaaacagggc cacttcagag ggtctcctgc cctgcagggc |
| 3541 | ctttctaccc gtgtccactg gaggggctgt ggccatcagc tctggctgtg taggggagga |
| 3601 | aggggtgcat gcatgtcccc caccctccac agtcttcctt gcctttagag tgaccctgca |
| 3661 | gagtcactca gccaaatctg tctgctgctc cctctcctca gccagttggg tgtgcgcaga |
| 3721 | gctgtcatag ggtccctttg tcagccccga gttcagcttc ccaaacacca gtgttggata |
| 3781 | ttctgtgatt gattttggtc ctcctccgct gtcccccaac acccaggaat gggaatctgg |
| 3841 | cttggttcga gataggagct tttctgtgtc ctaagccctt tcatgctagc aggaagactg |
| 3901 | aaagcaaggt ggcccagtgt ggggtcatag ggcttgatag acctggcact gcctatctgc |
| 3961 | acttccaggt gccccaccta tttatctgag cccacaggtg gaaaggggaa ctgcctcagt |
| 4021 | gagaacgggg ggacggggat gttaggaaaa atacagtaaa gttgcaatga agaggttcat |
| 4081 | gaagtatgtc cttgttcttt ttggaaactc tcggcaaagg gcaaaccagc aagtattgag |
| 4141 | ggtacccatc tagctacttg gggtcaggac ctcgtcagac caggttcgga tacaatcatc |
| 4201 | tgctcatccc aggaatagtt tcttggggga ctcactcact ggtgccagtt ctaagtcaga |
| 4261 | gacaaaattc cactgtctgt tccttttgct gtctgaactt tatgtgttac tcccttcctt |
| 4321 | tggtcttcac tctaatccct ggagtttgtg ggcttttggt tatgtttggt tagtagatat |
| 4381 | caccgcaatg ccctagaaca gctatgaagc agaataccat atggccacct ggacattggg |
| 4441 | acttgggaat tcactctcaa ctgggccatc catgttgtga tgcccttgaa gtaaaatgga |
| 4501 | gccagcagga gtaccttctg taaatgcatg tggcaaagtg ctatttatag ggtgcccagg |
| 4561 | gagccgctga tgtacaataa ccttgaggtc ccccatactg aaaactgacc aaggcctgtg |
| 4621 | cacaggtagc ccctcatgct gggctctgga ccatgagctg agtaggaagg atagcagagg |
| 4681 | ccaaccctga ccttcctgga agttgtttcc ttaacttgaa tgttgagctt cctctaaagc |
| 4741 | tttctcgtgt atgtcttctc catgccacta ctctgaggcc tcctgtgtta tgtgtgaaca |
| 4801 | gttgtcttta tgtgggaatg acgacttgat tgggagtaga gtctcaaggt cattcccctc |
| 4861 | ttccctcaag actctctgaa tgctgctcca ctgtcttttg tcttggaggt cactcagcag |
| 4921 | gttccttgca tttgctgcct ggatgtgcag ctggcaacag tgatgaattg gtcactgctc |
| 4981 | tttctctata actgggatag atgtcctgcc ttggggtcac taaaggggtg accttgttcc |
| 5041 | ttgctttatg agcccattag cactttggtt caaggggccc accaagtctt ggacgggaag |
| 5101 | gcgctactgg ttttattgcc caaggttttg ttattgcttc tcttctgtgt ccttctcttt |
| 5161 | gttcagtgaa gccaatatgt aagatactgt ttttgtcccc attcccctac tcctgagcta |
| 5221 | ggaggaaaaa atgtgaatct taccagcagt tccagccaac caagtgattc ttcttcattc |
| 5281 | ttgatgggga gaagtacata caaagtttgt tctgacaggg cgcggtggct cacgcctgta |
| 5341 | atcccagcgc tttgggaggc agaggcaggt ggatcacctg aggtcgggag ttcgagacca |
| 5401 | gcctgaccaa catggagata tcctgtctct actaaaaata caaaaaaatt agccaggcat |
| 5461 | ggtggcacgt gcctgtaatc ccagctactc gcaaggctga ggcaggagaa tcgcttgaac |
| 5521 | ctgggaggcg gaggttgcag tgagccaaga ttgcgccatt gcactccagc ctgggcaaca |
| 5581 | agagagaaac tctgtctcaa aa |

By “mapk39 polypeptide” is meant a protein having substantial identity to GenBank Accession No. NP_{—}149132 or a fragment thereof, and corresponds to SEQ ID NO: 16, shown below.

| | SEQ ID NO: 16 |
|---|---|
| 1 | mepsrallgc lasaaaaapp gedgagagae eeeeeeeeaa aavgpgelgc daplpywtav |
| 61 | feyeaagede ltlrlgdvve vlskdsqvsg degwwtgqln qrvgifpsny vtprsafssr |
| 121 | cqpggedpsc yppiqlleid faeltleeii giggfgkvyr afwigdevav kaarhdpded |
| 181 | isqtienvrq eaklfamlkh pniialrgvc lkepnlclvm efarggplnr vlsgkrippd |
| 241 | ilvnwavqia rgmnylhdea ivpiihrdlk ssnililqkv engdlsnkil kitdfglare |
| 301 | whrttkmsaa gtyawmapev irasmfskgs dvwsygvllw elltgevpfr gidglavayg |
| 361 | vamnklalpi pstcpepfak lmedcwnpdp hsrpsftnil dqlttieesg ffempkdsfh |
| 421 | clqdnwkhei qemfdqlrak ekelrtweee ltraalqqkn qeellrrreq elaereidil |
| 481 | erelniiihq lcqekprvkk rkgkfrksrl klkdgnrisl psdfqhkftv qasptmdkrk |
| 541 | slinsrsspp asptiiprlr aiqltpgess ktwgrssvvp keegeeeekr apkkkgrtwg |
| 601 | pgtlgqkela sgdegspqrr ekanglstps esphfhlglk slvdgykqws ssapnlvkgp |
| 661 | rsspalpgft slmemallaa swvvpidiee dedsegpgsg esrlqhspsq sylcipfprg |
| 721 | edgdgpssdg iheeptpvns atstpqltpt nslkrggahh rrcevallgc gavlaatglg |
| 781 | fdlleagkcq llpleepepp areekkrreg lfqrssrprr stsppsrklf kkeepmlllg |
| 841 | dpsasltlls lssisecnst rsllrsdsde ivvyempvsp veapplspct hnplvnvrve |
| 901 | rfkrdpnqsl tpthvtlttp sqpsshrrtp sdgalkpetl lasrspssng lspspgagml |
| 961 | ktpspsrdpg efprlpdpnv vfpptprrwn tqqdstlerp ktleflprpr psanrqrldp |
| 1021 | wwfvspshar stspanssst etpsnldscf asssstveer pglpallpfq agplpptert |
| 1081 | lldldaegqs qdstvplcra elnthrpapy eiqqefws |

### Inhibitory Nucleic Acids

In certain cases, it may be advantageous to inhibit the expression of certain cell adhesion molecules, for example, in order to promote growth of the cell in suspension.

Accordingly, in certain aspects of the invention, inhibitory nucleotides are used to inhibit the expression of cell adhesion molecules.

In certain preferred aspects, for example, the invention features a cell comprising an expression vector comprising a nucleic acid molecule encoding a siat7e, lama4, cdk13, cox15, egr1, or gas6 inhibitory nucleic acid molecule, and a virus.

Inhibitory nucleic molecules are not limited to only those listed above, and may be designed to any sialyltransferase, or any laminin. The design and testing of inhibitory oligonucleotides is known and easily performed by one of skill in the art. For example, on the world wide web, invitrogen.com offers oligonucletide design tools to the public.

Inhibitory nucleic acid molecules are nucleobase oligomers that inhibit the expression of a cdk13, siat7e, lama4, cox15, egr1, gas6, map3k9, or gap43 nucleic acid molecule or polypeptide. Such oligonucleotides can be used to generate cells having altered growth characteristics (e.g., altered cell-cell or cell-substrate adhesion, rate of proliferation, growth to particular cell density) that are desirable for certain applications, such as vaccine production and the production of recombinant therapeutic polypeptides. Such oligonucleotides include single and double stranded nucleic acid molecules (e.g., DNA, RNA, and analogs thereof) that bind a nucleic acid molecule that encodes a siat7e, lama4, cdk13, cox15, egr1 or gas6 polypeptide (e.g., antisense molecules, siRNA, shRNA) as well as nucleic acid molecules that bind directly to a siat7e, lama4, cdk13, cox15, egr1 or gas6polypeptide to modulate its biological activity (e.g., aptamers).

siRNA

Short twenty-one to twenty-five nucleotide double-stranded RNAs are effective at down-regulating gene expression (Zamore et al., Cell 101: 25-33; Elbashir et al., Nature 411: 494-498, 2001, hereby incorporated by reference). The therapeutic effectiveness of an siRNA approach in mammals was demonstrated in vivo by McCaffrey et al. (Nature 418: 38-39.2002).

Given the sequence of a target gene, siRNAs may be designed to inactivate that gene. Such siRNAs, for example, could be administered directly to an affected tissue, or administered systemically. The nucleic acid sequence of siat7e, lama4, cdk13, cox15, egr1 or gas6 gene can be used to design small interfering RNAs (siRNAs). The 21 to 25 nucleotide siRNAs may be used, for example, as therapeutics to treat a vascular disease or disorder.

The inhibitory nucleic acid molecules of the present invention may be employed as double-stranded RNAs for RNA interference (RNAi)-mediated knock-down of siat7e, lama4, cdk13, cox15, egr1 or gas6 expression. In one embodiment, siat7e, lama4, cdk13, cox15, egr1 or gas6 expression is reduced in a CHO or HEK cell. RNAi is a method for decreasing the cellular expression of specific proteins of interest (reviewed in Tuschl, Chembiochem 2:239-245, 2001; Sharp, Genes & Devel. 15:485-490, 2000; Hutvagner and Zamore, Curr. Opin. Genet. Devel. 12:225-232, 2002; and Hannon, Nature 418:244-251, 2002). The introduction of siRNAs into cells either by transfection of dsRNAs or through expression of siRNAs using a plasmid-based expression system is increasingly being used to create loss-of-function phenotypes in mammalian cells.

In one embodiment of the invention, double-stranded RNA (dsRNA) molecule is made that includes between eight and nineteen consecutive nucleobases of a nucleobase oligomer of the invention. The dsRNA can be two distinct strands of RNA that have duplexed, or a single RNA strand that has self-duplexed (small hairpin (sh)RNA). Typically, dsRNAs are about 21 or 22 base pairs, but may be shorter or longer (up to about 29 nucleobases) if desired. dsRNA can be made using standard techniques (e.g., chemical synthesis or in vitro transcription). Kits are available, for example, from Ambion (Austin, Tex.) and Epicentre (Madison, Wis.). Methods for expressing dsRNA in mammalian cells are described in Brummelkamp et al. Science 296:550-553, 2002; Paddison et al. Genes & Devel. 16:948-958, 2002. Paul et al. Nature Biotechnol. 20:505-508, 2002; Sui et al. Proc. Natl. Acad. Sci. USA 99:5515-5520, 2002; Yu et al. Proc. Natl. Acad. Sci. USA 99:6047-6052, 2002; Miyagishi et al. Nature Biotechnol. 20:497-500, 2002; and Lee et al. Nature Biotechnol. 20:500-505, 2002, each of which is hereby incorporated by reference. RNA Polymerase III promoters suitable for the expression of an siRNA in a mammalian cell include the well-characterized U6 and H1 promoters. U6 and H1 promoters are used to drive the expression of siRNAs in mammalian cells (Sui et al., Proc Natl Acad Sci USA 99, 5515-5520, 2002, Brummelkamp et al Science 296:550-553, 2002).

Antisense Oligonucleotides

Inhibitory nucleic acid molecules include antisense oligonucleotides that specifically hybridize with one or more siat7e, lama4, cdk13, cox15, egr1 or gas6 polynucleotides. The specific hybridization of the nucleobase oligomer with siat7e, lama4, cdk13, cox15, egr1 or gas6 polynucleotide (e.g., RNA, DNA) interferes with the normal function of that siat7e, lama4, cdk13, cox15, egr1 or gas6polynucleotide, reducing the amount of siat7e, lama4, cdk13, cox15, egr1 or gas6polypeptide produced.

The invention features a nucleobase oligomer of up to about 30 nucleobases in length. Desirably, when administered to a cell, the oligomer inhibits expression of siat7e, lama4, cdk13, cox15, egr1 or gas6. A nucleobase oligomer of the invention may also contain, e.g., an additional 20, 40, 60, 85, 120, or more consecutive nucleobases that are complementary to an siat7e, lama4, cdk13, cox15, egr1 or gas6 polynucleotide. The nucleobase oligomer (or a portion thereof) may contain a modified backbone. Phosphorothioate, phosphorodithioate, and other modified backbones are known in the art. The nucleobase oligomer may also contain one or more non-natural linkages.

Ribozymes

Catalytic RNA molecules or ribozymes that include an antisense siat7e, lama4, cdk13, cox15, egr1 or gas6 sequence of the present invention can be used to inhibit expression of a siat7e, lama4, cdk13, cox15, egr1 or gas6 nucleic acid molecule. The inclusion of ribozyme sequences within antisense RNAs confers RNA-cleaving activity upon them, thereby increasing the activity of the constructs. The design and use of target RNA-specific ribozymes is described in Haseloff et al., Nature 334:585-591. 1988, and U.S. Patent Application Publication No. 2003/0003469 A1, each of which is incorporated by reference.

Accordingly, the invention also features a catalytic RNA molecule that includes, in the binding arm, an antisense RNA having between eight and nineteen consecutive nucleobases. In preferred embodiments of this invention, the catalytic nucleic acid molecule is formed in a hammerhead or hairpin motif. Examples of such hammerhead motifs are described by Rossi et al., Aids Research and Human Retroviruses, 8:183, 1992. Example of hairpin motifs are described by Hampel et al., “RNA Catalyst for Cleaving Specific RNA Sequences,” filed Sep. 20, 1989, which is a continuation-in-part of U.S. Ser. No. 07/247,100 filed Sep. 20, 1988, Hampel and Tritz, Biochemistry, 28:4929, 1989, and Hampel et al., Nucleic Acids Research, 18: 299, 1990. These specific motifs are not limiting in the invention and those skilled in the art will recognize that all that is important in an enzymatic nucleic acid molecule of this invention is that it has a specific substrate binding site which is complementary to one or more of the target gene RNA regions, and that it have nucleotide sequences within or surrounding that substrate binding site which impart an RNA cleaving activity to the molecule.

Small hairpin RNAs consist of a stem-loop structure with optional 3′ UU-overhangs. While there may be variation, stems can range from 21 to 31 base pair (desirably 25 to 29 bp), and the loops can range from 4 to 30 bp (desirably 4 to 23 bp). For expression of shRNAs within cells, plasmid vectors containing either the polymerase III H1-RNA or U6 promoter, a cloning site for the stem-looped RNA insert, and a 4-5-thymidine transcription termination signal can be employed. The Polymerase III promoters generally have well-defined initiation and stop sites and their transcripts lack poly(A) tails. The termination signal for these promoters is defined by the polythymidine tract, and the transcript is typically cleaved after the second uridine. Cleavage at this position generates a 3′ UU overhang in the expressed shRNA, which is similar to the 3′ overhangs of synthetic siRNAs. Additional methods for expressing the shRNA in mammalian cells are described in the references cited above.

### Delivery of Nucleobase Oligomers

Naked inhibitory nucleic acid molecules, or analogs thereof, are capable of entering mammalian cells and inhibiting expression of a gene of interest. Nonetheless, it may be desirable to utilize a formulation that aids in the delivery of oligonucleotides or other nucleobase oligomers to cells (see, e.g., U.S. Pat. Nos. 5,656,611, 5,753,613, 5,785,992, 6,120,798, 6,221,959, 6,346,613, and 6,353,055, each of which is hereby incorporated by reference).

### Knockdown of Polypeptide Expression

As described in more detail below, cells having reduced expression of siat7e, lama4, cdk13, cox15, egr1 or gash have altered growth characteristics (e.g., altered cell-cell or cell-substrate adhesion, rate of proliferation, growth to particular cell density) that are desirable for certain applications, including vaccine production. Such cells are generated using any method known in the art. In one embodiment, a targeting vector is used that creates a knockout mutation in a gene of interest. The targeting vector is introduced into a suitable cell line to generate one or more cell lines that carry a knockout mutation. By a “knockout mutation” is meant an artificially-induced alteration in a nucleic acid molecule (created by recombinant DNA technology or deliberate exposure to a mutagen) that reduces the biological activity of the polypeptide normally encoded therefrom by at least about 50%, 75%, 80%, 90%, 95%, or more relative to the unmutated gene. The mutation can be, without limitation, an insertion, deletion, frameshift mutation, or a missense mutation. The targeting construct may result in the disruption of the gene of interest, e.g., by insertion of a heterologous sequence containing stop codons, or the construct may be used to replace the wild-type gene with a mutant form of the same gene, e.g. a “knock-in.” In another example, FRT sequences may be introduced into the cell such that they flank the gene of interest. Transient or continuous expression of the FLP protein is then used to induce site-directed recombination, resulting in the excision of the gene of interest. The use of the FLP/FRT system is well established in the art and is described in, for example, U.S. Pat. No. 5,527,695, and in Lyznik et al. (Nucleic Acid Research 24:3784-3789, 1996).

Furthermore, the targeting construct may contain a sequence that allows for conditional expression of the gene of interest. For example, a sequence may be inserted into the gene of interest that results in the protein not being expressed in the presence of tetracycline. Such conditional expression of a gene is described in, for example, Yamamoto et al. (Cell 101:57-66, 2000)).

Conditional knockout cells are also produced using the Cre-lox recombination system. Cre is an enzyme that excises DNA between two recognition sites termed loxP. The cre transgene may be under the control of an inducible, developmentally regulated, tissue specific, or cell-type specific promoter. In the presence of Cre, the gene, for example a nucleic acid sequence described herein, flanked by loxP sites is excised, generating a knockout. This system is described, for example, in Kilby et al. (Trends in Genetics 9:413-421, 1993).

### Viral Propagation

The cells of the present invention are extremely useful for the propagation of virus particles, for example influenza virus particles, because they may be grown at high density due to their altered growth characteristics. Inactivated viruses, viral polypeptides, and fragments thereof may be used in the production of prophylactic and therapeutic vaccines. Alternatively, cells of the invention may be used to produce viruses for use as vectors for gene therapy applications.

In one embodiment, the cells of the invention are MDCK cells. If desired, one skilled in the art appreciates that the compositions and methods of the invention employs virtually any other cells that are amenable for viral infection and growth in suspension due to their expression of a siat7e, lama4, cdk13, cox15, egr1 or gas6 inhibitory nucleic acid molecule or polypeptide. For instance, the cell can be a Vero cell. The Vero cell line is derived from kidney epithelial cells of the African Green Monkey. Studies have indicated that the Vero line is a suitable system for the primary isolation and cultivation of influenza A viruses (E. A. Govorkova, N. V. Kaverin, L. V. Gubareva, B. Meignier, and R. G. Webster, J. Infect. Dis. 172:250-253, 1995), and further that Vero cells are suitable for isolation and productive replication of influenza A and B viruses (Govorkova et al. J. Virol. 1996 August; 70(8): 5519-5524).

In certain preferred examples, the cells of the invention comprise an expression vector. The expression vector can comprise a nucleic acid molecule encoding a polypeptide or inhibitory nucleic acid molecule selected from the group consisting of, but not limited to, cdk13, siat7e, lama4, cox15, egr1, gas6, map3k9, and gap43, and a virus.

In other certain examples, the cell can comprise an expression vector comprising a nucleic acid molecule encoding, for example, a sialyltransferase or a laminin inhibitory nucleic acid molecule. In other example, the cell can comprise an expression vector comprising a nucleic acid molecule encoding, for example, a siat7e, lama4, cdk13, cox15, egr1, or gas6 inhibitory nucleic acid molecule, and a virus. In certain cases, the cell expresses an increased level of a siat7e, lama4, cdk13, cox15, egr1, or gas6 nucleic acid molecule or polypeptide relative to a control cell. In other certain cases, the cell expresses a decreased level of a siat7e, lama4, cdk13, cox15, egr1, or gas6 nucleic acid molecule or polypeptide relative to a control cell.

More specifically, the cell may express an increased level of siat7e nucleic acid molecule or polypeptide relative to a control cell. In other examples, the cell may express a decreased level of lama4 nucleic acid molecule or polypeptide relative to a control cell.

The invention also features cells that comprise a mutation that alters the expression or activity of a polypeptide selected from the group consisting of cdk13, siat7e, lama4, cox15, egr1, gas6, map3k9, and gap43 polypeptide, and a virus.

Any mutation that alters the expression of the polypeptide is appropriate according to the invention, however in certain cases the mutation is a deletion, missense mutation, or frameshift.

Cells of the invention as described herein may be cultured in suspension. For instance, cells can be cultures in spinner flasks in suspension. In some cases, attached lines that have been adapted to growth in suspension are cultured in spinner flasks. Spinner flasks are either plastic or glass bottles with a central magnetic stirrer shaft and side arms for the addition and removal of cells and medium, and gassing with CO2 enriched air. Inoculated spinner flasks are placed on a stirrer and incubated under the culture conditions appropriate for the cell line. Cultures should be stirred at 100-250 revolutions per minute. Spinner flask systems designed to handle culture volumes of 1-12 liters are available commercially.

It is also possible to culture the cells in a bioreactor. Numerous cell culture bioreactors are commercially available that provide culture bioreactors for research and development through production applications. Bioreactors are suitable for mammalian, animal, plant, algae, and insect cell culture. Culturing cells in a bioreactor provides for cell culture at high volume, for example at 1 L or more volumes, and thus provides high yield of viral product.

In preferred embodiments, the bioreactor is a wave bioreactor. The wave bioreactor is a cell culture system for 0.1 to 500 liters. Using the wave bioreactor, the culture medium and cells only contact a presterile, disposable chamber that is placed on a special rocking platform. The rocking motion of this platform induces waves in the culture fluid. These waves provide mixing and oxygen transfer, resulting in a perfect environment for cell growth that can easily support over 10×106 cells/ml.

Other bioreactors are known in the art, for example those described by U.S. Pat. No. 6,943,08 and U.S. Pat. No. 7,198,940, both references are incorporated in their entireties herein.

Cells that are cultured by the methods of the invention as described herein have characteristics that are different from or altered from control cells. For instance, cells cultured by the methods of the invention may have altered growth characteristics relative to a control cell, such as increased or decreased adhesive characteristics. Adhesive characteristics may be measured by cell aggregation or in a shear flow chamber. The altered growth characteristics may be, but are not limited to, increased cell density or an increased cell population size relative to a control cell.

The cells of the invention as described herein have applications in producing immunogenic compositions, vaccines, viruses. When cultured, for example when cultured in suspension, the cells of the invention may express increased levels of an immunogenic composition relative to a control cell. The cells of the invention may express increased levels of a vaccine relative to a control cell. The cells of the invention may express increased levels of a virus relative to a control cell.

### Viruses

As described herein, the invention features cells for viral propagation having modified growth characteristics that allow them to be grown to high density and to grow in suspension. The modified growth characteristics are related to cell's expression of a recombinant polypeptide selected from the group consisting of: cdk13, siat7e, lama4, cox15, egr1, gas6, map3k9, and gap43, or a nucleic acid molecule encoding a siat7e, lama4, cdk13, cox15, egr1, or gas6 inhibitory nucleic acid molecule. The invention also features, as described herein, methods of producing a vaccine or an immunogenic composition comprising a virus, methods of producing a vaccine or an immunogenic composition comprising infecting a cell with a virus.

In certain embodiments of the invention, the cells that comprise the expression vector and the virus, or the cells that are infected with the virus are MDCK cells. MDCK cells are susceptible to viruses selected from, but not limited to: Coxsackievirus B5vesicular stomatitis (Indiana); vaccinia; coxsackievirus B5; reovirus 2, 3; adenovirus 4, 5; vesicular exanthema of swine; infectious canine hepatitis Reovirus type 2vesicular stomatitis (Indiana); vaccinia; coxsackievirus B5; reovirus 2, 3; adenovirus 4, 5; vesicular exanthema of swine; infectious canine hepatitis Adeno-associated virus 4vesicular stomatitis (Indiana); vaccinia; coxsackievirus B5; reovirus 2, 3; adenovirus 4, 5; vesicular exanthema of swine; infectious canine hepatitis Vaccinia virusvesicular stomatitis (Indiana); vaccinia; coxsackievirus B5; reovirus 2, 3; adenovirus 4, 5; vesicular exanthema of swine; infectious canine hepatitis Vesicular stomatitis virusvesicular stomatitis (Indiana); vaccinia; coxsackievirus B5; reovirus 2, 3; adenovirus 4, 5; vesicular exanthema of swine; infectious canine hepatitis Adeno-associated virus 5vesicular stomatitis (Indiana); vaccinia; coxsackievirus B5; reovirus 2, 3; adenovirus 4, 5; vesicular exa. Information about MDCK cell virus susceptibility is publicly available on the world wide web at http://www.atcc.org/ATCCAdvancedCatalogSearch/ProductDetails/tabid/452/Default.aspx? ATCCNum=CCL-34&Template=cellBiology.

In certain examples, the virus is a virus that has been found to infect humans. Examples of viruses that have been found in humans include but are not limited to Retroviridae (e.g. human immunodeficiency viruses, such as HIV-1 (also referred to as HDTV-III, LAVE or HTLV-III/LAV, or HIV-III; and other isolates, such as HIV-LP; Picornaviridae (e.g. polio viruses, hepatitis A virus; enteroviruses, human Coxsackie viruses, rhinoviruses, echoviruses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviridae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, orbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus (HSV)₁ and 2, varicella zoster virus, cytomegalovirus (CMV), herpes virus; Poxyiridae (variola viruses, vaccinia viruses, pox viruses); and Iridoviridae (e.g. African swine fever virus); and unclassified viruses (e.g. the agent of delta hepatitis (thought to be a defective satellite of hepatitis B virus), the agents of non-A, non-B hepatitis (class 1=internally transmitted; class 2=parenterally transmitted (i.e. Hepatitis C); Norwalk and related viruses, and astroviruses).

In other certain examples, the virus in a influenza virus, and more particularly a human influenza virus. Influenza viruses include, but are not limited to, Influenza A H1N1, H3N2, H5N1, Influenza B, and West Nile virus.

The mature influenza virus contains both HA and NA proteins in its outer envelope. The HA is present as trimers. Each HA monomer consists of two polypeptides (HA1 and HA2) linked by a disulfide bond. These polypeptides are derived by cleavage of a single precursor protein, HA0, during maturation of the influenza virus. In part, because these molecules are tightly folded, the HA0 and the mature HA1 and HA2 differ slightly in their conformation and antigenic characteristics. Furthermore, the HA0 is more stable and resistant to denaturation and to proteolysis. Recently it has been reported that a baculovirus/insect cell culture derived recombinant HA0 conferred protective immunity to influenza (Wilkinson, B., MicroGeneSys Recombinant Influenza Vaccine, PMA/CBER Viral Influenza Meeting, Dec. 8, 1994). One limitation of recombinant HA0 vaccines is their inability to stimulate immune responses against non-HA antigens that may provide greater and more durable protection, especially for high-risk populations that do not respond well to immunization.

Influenza A viruses possess a genome of eight single-stranded negative-sense viral RNAs (vRNAs) that encode a total of ten proteins. The influenza virus life cycle begins with binding of the hemagglutinin (HA) to sialic acid-containing receptors on the surface of the host cell, followed by receptor-mediated endocytosis. The low pH in late endosomes triggers a conformational shift in the HA, thereby exposing the N-terminus of the HA2 subunit (the so-called fusion peptide). The fusion peptide initiates the fusion of the viral and endosomal membrane, and the matrix protein (M1) and RNP complexes are released into the cytoplasm. RNPs consist of the nucleoprotein (NP), which encapsidates vRNA, and the viral polymerase complex, which is formed by the PA, PB1, and PB2 proteins. RNPs are transported into the nucleus, where transcription and replication take place. The RNA polymerase complex catalyzes three different reactions: synthesis of an mRNA with a 5′ cap and 3′ polyA structure, of a full-length complementary RNA (cRNA), and of genomic vRNA using the cDNA as a template. Newly synthesized vRNAs, NP, and polymerase proteins are then assembled into RNPs, exported from the nucleus, and transported to the plasma membrane, where budding of progeny virus particles occurs. The neuraminidase (NA) protein plays a crucial role late in infection by removing sialic acid from sialyloligosaccharides, thus releasing newly assembled virions from the cell surface and preventing the self aggregation of virus particles. Although virus assembly involves protein-protein and protein-vRNA interactions, the nature of these interactions is largely unknown.

Although influenza B and C viruses are structurally and functionally similar to influenza A virus, there are some differences. For example, influenza B virus does not have a M2 protein. Similarly, influenza C virus does not have a M2 protein. In certain preferred embodiments, the virus is an adenovirus.

### Vaccine Production

The invention also provides for a method of inducing an immunological response in an individual, particularly a human, which comprises inoculating the individual with a composition of the invention (e.g., a virus or adenovirus), in a suitable carrier for the purpose of inducing an immune response to protect said individual from infection with the virus or adenovirus. The administration of this immunological composition may be used either therapeutically in individuals already experiencing the viral or adenoviral infection, or may be used prophylactically to prevent the viral or adenoviral infection.

Therapeutic vaccines may reduce or alleviate a symptom associated with a viral or adenoviral infection, such as the severity of influenza. In some cases, a therapeutic vaccine will enhance the immune response of an individual infected with the virus. For example, the vaccines of the invention are useful for reducing the frequency or severity of symptomatic or asymptomatic influenza outbreaks. Symptomatic outbreaks are characterized by the appearance of influenza symptoms or other clinical symptoms of infection.

Prophylactic vaccines may be used to prevent or reduce the probability that a subject (e.g., a human) will be infected with a virus, for example an influenza virus. Most advantageously, a vaccine prevents the transmission of the virus from an infected individual to an uninfected individual. Also useful in the methods of the invention are vaccines that prevent the virus from establishing a latent infection in a virus infected subject.

Also useful as therapeutic or prophylactic vaccines are cellular vaccines, which contain cells infected with a virus with a mutation. Preferably, such vaccines include a cell (e.g., a dendritic cell) derived from the subject that requires vaccination. In general, the cell is obtained from a biological sample of the subject, such as a blood sample. Preferably, a dendritic cell or dendritic stem cell is obtained from the subject, and the cell is cultured in vitro to obtain a population of dendritic cells. The cultured cells are infected with a mutant virus. The infected cells are then re-introduced into the subject where they enhance or elicit an immune response against a wild-type virus.

The preparation of vaccines that contain immunogenic polypeptides is known to one skilled in the art. The polypeptide may serve as an antigen for vaccination, or an expression vector encoding the polypeptide, or fragments or variants thereof, might be delivered in vivo in order to induce an immunological response comprising the production of antibodies or a T cell immune response.

In certain embodiments, the invention features methods of producing a vaccine or immunogenic composition that comprise isolating a virus from a virus infected cell, the cell comprising an expression vector comprising a nucleic acid molecule encoding a polypeptide selected from the group consisting of: cdk13, siat7e, lama4, cox15, egr1, gas6, map3k9, and gap43, and thereby producing a vaccine or an immunogenic composition.

In related embodiments, the invention features methods of producing a vaccine or an immunogenic composition in a cell comprising infecting a cell comprising an expression vector comprising a nucleic acid molecule encoding a polypeptide such as a sialyltransferase or a laminin, or in preferred embodiments a polypeptide selected from the group consisting of cdk13, siat7e, lama4, cox15, egr1, gas6, map3k9, and gap43 or a nucleic acid molecule encoding a siat7e, lama4, cdk13, cox15, egr1, or gas6 inhibitory nucleic acid molecule with a virus producing virus in the cell, and harvesting the virus, thereby producing a vaccine in the cell.

The method of producing a vaccine or an immunogenic composition in a cell can comprise infecting a cell, wherein the cell comprises a mutation that alters the expression or activity of a polypeptide selected from the group consisting of cdk13, siat7e, lama4, cox15, egr1, gas6, map3k9, and gap43 polypeptide with a virus, producing virus in the cell; and harvesting the virus, thereby producing a virus or an immunogenic composition in the cell.

The cell can be any cell that is capable of viral infection and growth in suspension, and that is able to produce the virus or immunogenic composition; however preferred cells for use in the invention are MDCK cells.

In certain examples, the method further comprises the step of inactivating the virus. Viral inactivation provides the virus in a non-active form. Any method of inactivation is possible according to the methods of the invention; however in certain preferred embodiments, the viral inactivation is heat inactivation.

Inactivated virus vaccines and immunogenic compositions of the invention are provided by inactivating replicated virus of the invention using known methods, such as, but not limited to, formalin or .beta.-propiolactone treatment. Inactivated vaccine types that can be used in the invention can include whole-virus (WV) vaccines or subvirion (SV) (split) vaccines. The WV vaccine contains intact, inactivated virus, while the SV vaccine contains purified virus disrupted with detergents that solubilize the lipid-containing viral envelope, followed by chemical inactivation of residual virus.

Other attenuating mutations can be introduced into influenza virus genes by site-directed mutagenesis to rescue infectious viruses bearing these mutant genes. Attenuating mutations can be introduced into non-coding regions of the genome, as well as into coding regions. Such attenuating mutations can also be introduced into genes other than the HA or NA, e.g., the PB2 polymerase gene (Subbarao et al., 1993). Thus, new donor viruses can also be generated bearing attenuating mutations introduced by site-directed mutagenesis, and such new donor viruses can be used in the reduction of live attenuated reassortants H1N1 and H3N2 vaccine candidates in a manner analogous to that described above for the A/AA/6/60 ca donor virus. Similarly, other known and suitable attenuated donor strains can be reasserted with influenza virus of the invention to obtain attenuated vaccines suitable for use in the vaccination of mammals (Enami et al., 1990; Muster et al., 1991; Subbarao et al., 1993).

It is preferred that such attenuated viruses maintain the genes from the virus that encode antigenic determinants substantially similar to those of the original clinical isolates. This is because the purpose of the attenuated vaccine is to provide substantially the same antigenicity as the original clinical isolate of the virus, while at the same time lacking infectivity to the degree that the vaccine causes minimal change of inducing a serious pathogenic condition in the vaccinated mammal.

The virus can thus be attenuated or inactivated, formulated and administered, according to known methods, as a vaccine to induce an immune response in an animal, e.g., a mammal. Methods are well-known in the art for determining whether such attenuated or inactivated vaccines have maintained similar antigenicity to that of the clinical isolate or high growth strain derived therefrom. Such known methods include the use of antisera or antibodies to eliminate viruses expressing antigenic determinants of the donor virus; chemical selection (e.g., amantadine or rimantidine); HA and NA activity and inhibition; and DNA screening (such as probe hybridization or PCR) to confirm that donor genes encoding the antigenic determinants (e.g., HA or NA genes) are not present in the attenuated viruses. See, e.g., Robertson et al., 1988; Kilbourne, 1969; Aymard-Henry et al., 1985; Robertson et al., 1992.

Live, attenuated influenza virus vaccines, can also be used for preventing or treating influenza virus infection, according to known method steps. Attenuation is preferably achieved in a single step by transfer of attenuated genes from an attenuated donor virus to a replicated isolate or reasserted virus according to known methods (see, e.g., Murphy, 1993). Since resistance to influenza A virus is mediated by the development of an immune response to the HA and NA glycoproteins, the genes coding for these surface antigens must come from the reassorted viruses or high growth clinical isolates. The attenuated genes are derived from the attenuated parent. In this approach, genes that confer attenuation preferably do not code for the HA and NA glycoproteins. Otherwise, these genes could not be transferred to reabsortants bearing the surface antigens of the clinical virus isolate.

### Therapeutic and Prophylactic Methods

The administration of the compositions of the invention (or the antisera that it elicits) may be for either a “prophylactic” or “therapeutic” purpose. When provided prophylactically, the compositions of the invention (e.g. vaccines or immunogenic compositions), may be provided before or at the onset or at the early stages of any symptom of a pathogen infection. In certain preferred examples, the prophylactic administration of the composition serves to prevent or attenuate any subsequent infection.

When provided prophylactically, immunogenic compositions of the invention are provided before, or at the onset or at the early stages of any symptom of a disease becomes manifest. The prophylactic administration of the composition serves to prevent or attenuate one or more symptoms associated with the disease.

When provided therapeutically, an attenuated or inactivated viral vaccine is provided upon the detection of a symptom of actual infection. The therapeutic administration of the compound(s) serves to attenuate any actual infection.

When provided therapeutically, a gene therapy composition is provided upon the detection of a symptom or indication of the disease. The therapeutic administration of the compound(s) serves to attenuate a symptom or indication of that disease.

The protection provided by the immunogenic composition or vaccine need not be absolute, i.e., the viral (e.g. influenza) infection need not be totally prevented or eradicated, if there is a significant improvement compared with a control population or set of patients. Protection may be limited to mitigating the severity or rapidity of onset of symptoms of the virus infection.

In certain examples, the invention features methods of producing an immune response in a subject comprising administering to the subject a pharmaceutical composition of the invention as described herein, in an amount sufficient to generate an immune response, and thereby producing an immune response in a subject.

In other certain examples, the invention features a method of treating or preventing a subject suffering from a viral infection comprising administering to the subject a pharmaceutical composition of the invention as described herein, in an amount sufficient to generate an immune response, and thereby treating a subject suffering from a viral infection.

The immune response can be a protective immune response, or a cell-mediated immune response. The immune response may be a humoral immune response. The immune response that is generated may be both a cell-mediated immune response and a humoral immune response.

In certain cases, the invention may comprise isolating immune cells from the subject; and testing an immune response of the isolated immune cells in vitro.

### Recombinant Polypeptide Expression

Methods for expressing a recombinant polypeptide, such as a therapeutic biological polypeptide or immunogenic polypeptide, involve the transfection of cells of the invention (e.g., a cell comprising an expression vector comprising a nucleic acid molecule encoding a siat7e, lama4, cdk13, cox15, egr or gas6 nucleic acid molecule or a cell comprising an expression vector comprising a nucleic acid molecule encoding a siat7e, lama4, cdk13, cox15, egr or gas6 inhibitory nucleic acid molecule) with a nucleic acid molecule encoding a recombinant protein, variant, or fragment thereof. Such nucleic acid molecules can be delivered to cells in vitro or to the cells of a subject having a disease or disorder amenable to treatment with the recombinant polypeptide. The nucleic acid molecules must be delivered to the cells in a form in which they can be taken up so that therapeutically effective levels of the protein or a fragment thereof can be produced.

Transducing viral (e.g., retroviral, adenoviral, and adeno-associated viral) vectors can be used for polynucleotide expression, especially because of their high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). For example, a polynucleotide encoding a therapeutic protein, variant, or a fragment thereof, can be cloned into a retroviral vector and expression can be driven from its endogenous promoter, from the retroviral long terminal repeat, or from a promoter specific for a target cell type of interest. Other viral vectors that can be used include, for example, a vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; Le Gal La Salle et al., Science 259:988-990, 1993; and Johnson, Chest 107:77 S-83S, 1995). Retroviral vectors are particularly well developed and have been used in clinical settings (Rosenberg et al., N. Engl. J. Med 323:370, 1990; Anderson et al., U.S. Pat. No. 5,399,346). For some applications, a viral vector is used to administer a polynucleotide.

Non-viral approaches can also be employed for the introduction of therapeutic to a cell where recombinant protein expression is desired. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Preferably the nucleic acid molecules are administered in combination with a liposome and protamine.

Gene transfer can also be achieved using non-viral means involving transfection in vitro. Such methods include the use of calcium phosphate, DEAE dextran, electroporation, and protoplast fusion. Liposomes can also be potentially beneficial for delivery of DNA into a cell. Transplantation of normal genes into the affected tissues of a patient can also be accomplished by transferring a normal nucleic acid into a cultivatable cell type ex vivo (e.g., an autologous or heterologous primary cell or progeny thereof), after which the cell (or its descendants) are injected into a targeted tissue.

cDNA expression of a recombinant protein can be directed from any suitable promoter (e.g., the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

Polypeptides and Analogs

Also included in the invention are recombinant polypeptides or fragments thereof that are modified in ways that enhance or inhibit their ability to be expressed by a cell of the invention. The invention provides methods for altering an amino acid sequence or nucleic acid sequence by producing an alteration in the sequence. Such alterations may include certain mutations, deletions, insertions, or post-translational modifications. The invention further includes analogs of any naturally-occurring polypeptide of the invention. Analogs can differ from a naturally-occurring polypeptide of the invention by amino acid sequence differences, by post-translational modifications, or by both. Analogs of the invention will generally exhibit at least 85%, more preferably 90%, and most preferably 95% or even 99% identity with all or part of a naturally-occurring amino, acid sequence of the invention. The length of sequence comparison is at least 5, 10, 15 or 20 amino acid residues, preferably at least 25, 50, or 75 amino acid residues, and more preferably more than 100 amino acid residues. Again, in an exemplary approach to determining the degree of identity, a BLAST program may be used, with a probability score between e⁻³ and e⁻¹⁰⁰ indicating a closely related sequence. Modifications include in vivo and in vitro chemical derivatization of polypeptides, e.g., acetylation, carboxylation, phosphorylation, or glycosylation; such modifications may occur during polypeptide synthesis or processing or following treatment with isolated modifying enzymes. Analogs can also differ from the naturally-occurring polypeptides of the invention by alterations in primary sequence. These include genetic variants, both natural and induced (for example, resulting from random mutagenesis by irradiation or exposure to ethanemethylsulfate or by site-specific mutagenesis as described in Sambrook, Fritsch and Maniatis, Molecular Cloning: A Laboratory Manual (2d ed.), CSH Press, 1989, or Ausubel et al., supra). Also included are cyclized peptides, molecules, and analogs which contain residues other than L-amino acids, e.g., D-amino acids or non-naturally occurring or synthetic amino acids, e.g., .beta. or .gamma. amino acids.

In addition to full-length polypeptides, the invention also includes fragments of any one of the polypeptides of the invention. As used herein, the term “a fragment” means at least 5, 10, 13, or 15. In other embodiments a fragment is at least 20 contiguous amino acids, at least 30 contiguous amino acids, or at least 50 contiguous amino acids, and in other embodiments at least 60 to 80 or more contiguous amino acids. Fragments of the invention can be generated by methods known to those skilled in the art or may result from normal protein processing (e.g., removal of amino acids from the nascent polypeptide that are not required for biological activity or removal of amino acids by alternative mRNA splicing or alternative protein processing events).

Analogs have a chemical structure designed to mimic the reference proteins functional activity. Such analogs are administered according to methods of the invention. Protein analogs may exceed the physiological activity of the original polypeptide. Methods of analog design are well known in the art, and synthesis of analogs can be carried out according to such methods by modifying the chemical structures such that the resultant analogs increase the therapeutic activity of a reference polypeptide. These chemical modifications include, but are not limited to, substituting alternative R groups and varying the degree of saturation at specific carbon atoms of a reference fusion polypeptide. Preferably, the fusion protein analogs are relatively resistant to in vivo degradation, resulting in a more prolonged therapeutic effect upon administration. Assays for measuring functional activity include, but are not limited to, those described in the Examples below.

### Immunogenic Compositions and Vaccine Administration

Compositions of the present invention are produced by any of the methods of the invention as described herein.

For instance, the invention described methods of producing a vaccine or immunogenic composition that comprise isolating a virus from the cells as described herein, and incorporating an effective amount of the virus into a pharmaceutically acceptable excipient.

Pharmaceutical compositions of the present invention, suitable for inoculation or for administration, comprise immunogenic compositions produced by the methods as described herein, viruses produced by the methods as described herein, and optionally further comprising a pharmaceutically acceptable carrier, for example a sterile aqueous or non-aqueous solutions, suspensions, and emulsions. The compositions can further comprise auxiliary agents or excipients, as known in the art. See, e.g., Berkow et al., 1987; Avery's Drug Treatment, 1987; Osol, 1980; Katzung, 1992. The composition of the invention is generally presented in the form of individual doses (unit doses).

The immunogenic compositions are capable of generating a protective immune response to a virus or pathogen when administered to a mammal. In preferred embodiments, the response is a humoral response.

A pharmaceutical composition according to the present invention may further or additionally comprise another agent or compound, for example, for gene therapy, immunosuppressants, anti-inflammatory agents or immune enhancers, and for vaccines, chemotherapeutics including, but not limited to, gamma globulin, amantadine, guanidine, hydroxybenzimidazole, interferon-o, interferon-.beta., interferon-.gamma., tumor necrosis factor-alpha, thiosemicarbarzones, methisazone, rifampin, ribavirin, a pyrimidine analog, a purine analog, foscarnet, phosphonoacetic acid, acyclovir, dideoxynucleosides, a protease inhibitor, or ganciclovir.

The composition can also contain variable but small quantities of endotoxin-free formaldehyde, and preservatives, which have been found safe and not contributing to undesirable effects in the organism to which the composition is administered.

Formulation of the viruses of the invention can be carried out using methods that are standard in the art. Numerous pharmaceutically acceptable solutions for use in vaccine preparation are well known and can readily be adapted for use in the present invention by those of skill in this art (see, e.g., Remington's Pharmaceutical Sciences (18th edition), ed. A. Gennaro, 1990, Mack Publishing Co., Easton, Pa.). For example, the viruses can be diluted in a physiologically acceptable solution, such as sterile saline or sterile buffered saline. In another example, the viruses can be administered and formulated, for example, as a clarified suspension, or a fluid harvested from cell cultures infected with the virus.

The immunogenic compositions and vaccines of the invention can be administered using methods that are well known in the art, and appropriate amounts of the vaccines to be administered can readily be determined by those of skill in the art. What is determined to be an appropriate amount of virus to administer can be determined by consideration of factors such as, e.g., the size and general health of the subject to whom the virus is to be administered. For example, the viruses of the invention can be formulated as sterile aqueous solutions containing between 10² and 10⁸, e.g., 10³ to 10⁷ or 10⁴ to 10⁶, infectious units (e.g., plaque-forming units or tissue culture infectious doses) in a dose volume of 0.1 to 1.0 ml, to be administered by, for example, intramuscular, subcutaneous, or intradermal routes. In addition, because certain viruses (e.g., flaviviruses) may be capable of infecting the human host via mucosal routes, such as the oral route (Gresikova et al., “Tick-borne Encephalitis,” In The Arboviruses, Ecology and Epidemiology, Monath (ed.), CRC Press, Boca Raton, Fla., 1988, Volume IV, 177-203), the viruses can be administered by mucosal (e.g., oral) routes as well. Solid forms suitable for injection may also be prepared as emulsions, or with the polypeptides encapsulated in liposomes.

In certain preferred examples, the mode of administration is selected from the group consisting of topical administration, oral administration, injection by needle, needleless jet injection, intradermal administration, intramuscular administration, and gene gun administration.

Further, the vaccines of the invention can be administered in a single dose or, optionally, administration can involve the use of a priming dose followed by one or more booster doses that are administered, e.g., 2-6 months later, as determined to be appropriate by those of skill in the art.

Vaccine antigens are usually combined with a pharmaceutically acceptable carrier, which includes any carrier that does not induce the production of antibodies harmful to the individual receiving the carrier. Suitable carriers typically comprise large macromolecules that are slowly metabolized, such as proteins, polysaccharides, polylactic acids, polyglycolic acids, polymeric amino acids, amino acid copolymers, lipid aggregates, and inactive virus particles. Such carriers are well known to those skilled in the art. These carriers may also function as adjuvants.

In certain examples, the compositions of the invention may also include an adjuvant; adjuvants that are known to those skilled in the art can be used in the administration of the viruses of the invention. Adjuvants are immunostimulating agents that enhance vaccine effectiveness. Effective adjuvants include, but are not limited to, aluminum salts such as aluminum hydroxide and aluminum phosphate, muramyl peptides, bacterial cell wall components, saponin adjuvants, and other substances that act as immunostimulating agents to enhance the effectiveness of the composition.

Optionally, an adjuvant may be administered as a second agent in addition to the compositions of the invention. Adjuvants that can be used to enhance the immunogenicity of the viruses include, for example, liposomal formulations, synthetic adjuvants, such as (e.g., QS21), muramyl dipeptide, monophosphoryl lipid A, or polyphosphazine. Although these adjuvants are typically used to enhance immune responses to inactivated vaccines, they can also be used with live vaccines. In the case of a virus delivered via a mucosal route, for example, orally, mucosal adjuvants such as the heat-labile toxin of E. coli (LT) or mutant derivations of LT can be used as adjuvants. In addition, genes encoding cytokines that have adjuvant activities can be inserted into the viruses. Thus, genes encoding cytokines, such as GM-CSF, IL-2, IL-12, IL-13, or IL-5, can be inserted together with foreign antigen genes to produce a vaccine that results in enhanced immune responses, or to modulate immunity directed more specifically towards cellular, humoral, or mucosal responses. Additional adjuvants that can optionally be used in the invention include toll-like receptor (TLR) modulators.

Immunogenic compositions also typically contain diluents, such as water, saline, glycerol, ethanol. Auxiliary substances may also be present, such as wetting or emulsifying agents, pH buffering substances, and the like. Proteins may be formulated into the vaccine as neutral or salt forms. The vaccines are typically administered parenterally, by injection; such injection may be either subcutaneously or intramuscularly. Additional formulations are suitable for other forms of administration, such as by suppository or orally. Oral compositions may be administered as a solution, suspension, tablet, pill, capsule, or sustained release formulation.

In addition, the vaccine can also be administered to individuals to generate polyclonal antibodies (purified or isolated from serum using standard methods) that may be used to passively immunize an individual. These polyclonal antibodies can also serve as immunochemical reagents.

In addition, it is possible to prepare live attenuated microorganism vaccines that express recombinant polypeptides. Suitable attenuated microorganisms are known in the art, and include, for example, viruses and bacteria.

According to the present invention, an “effective amount” of a composition is one that is sufficient to achieve a desired biological effect. It is understood that the effective dosage will be dependent upon the age, sex, health, and weight of the recipient, kind of concurrent treatment, if any, frequency of treatment, and the nature of the effect wanted. The ranges of effective doses provided below are not intended to limit the invention and represent preferred dose ranges. However, the most preferred dosage will be tailored to the individual subject, as is understood and determinable by one of skill in the art.

Vaccines are administered in a manner compatible with the dose formulation. The immunogenic composition or the vaccine comprises an immunologically effective amount of the antigenic polypeptides and other previously mentioned components. By an immunologically effective amount is meant a single dose, or a vaccine administered in a multiple dose schedule, that is effective for the treatment or prevention of an infection. The dose administered will vary, depending on the subject to be treated, the subject's health and physical condition, the capacity of the subject's immune system to produce antibodies, the degree of protection desired, and other relevant factors. Precise amounts of the active ingredient required will depend on the judgement of the skilled practitioner, but typically range between 2 ug to 500 ug, preferably 5 ug to 250 ug, of antigen per dose.

### Kits

The invention provides kits featuring immunogenic compositions for the treatment or prevention of a viral infection, particularly viral influenza. The kits of the invention can also be used in methods of gene therapy to provide viruses used to deliver a therapeutic polypeptide. In one embodiment, the kit includes a therapeutic or prophylactic composition containing an effective amount of an inactivated virus or fragments thereof (e.g., influenza virus) in unit dosage form. In some embodiments, the kit comprises a sterile container which contains a therapeutic or prophylactic viral composition; such containers can be boxes, ampoules, bottles, vials, tubes, bags, pouches, blister-packs, or other suitable container forms known in the art. Such containers can be made of plastic, glass, laminated paper, metal foil, or other materials suitable for holding medicaments.

If desired the immunogenic compositions of the invention are provided together with instructions for administering the composition to a subject having or at risk of developing a viral infection. The instructions will generally include information about the use of the composition for the treatment or prevention of a viral infection. In other embodiments, the instructions include at least one of the following: description of the immunogenic composition; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

### EXAMPLES

It should be appreciated that the invention should not be construed to be limited to the examples that are now described; rather, the invention should be construed to include any and all applications provided herein and all equivalent variations within the skill of the ordinary artisan.

### Example 1

### Characterization of Genetically Modified MDCK Cell Line Cultivated in Suspension for its Support of Influenza Virus Replication

As described herein, the ability to modify cellular properties such as adhesion is of interest in the design and performance of biotechnology-related processes. Further, the strategy of applying bioinformatics techniques to characterize and manipulate phenotypic behaviors represents a potentially powerful tool for altering the properties of various cell lines.

In other work by the present inventors, the transcription profiles of anchorage-dependent and anchorage-independent HeLa cells was compared using DNA microarrays (1). The gene siat7e (ST6GalNac V) was identified as one of the genes that plays a role in controlling the degree of cell adhesion. The gene expression profile of two phenotypically distinct, anchorage-dependent and anchorage-independent, HeLa cell lines were compared. With the aid of several statistical methods, two genes, siat7e, and lama4 were identified as potential targets for further study. The human sialytransferase, siat7e, is a type II transmembrane glycosylating enzyme that catalyzes the transfer of sialic acid from CMP-Neu5Ac to both glycoproteins and glycolipids. The gene lama4 encodes laminin alpha4, a member of the laminin family of glycoproteins often associated with adhesion. Experiments were carried out to investigate the separate effects of altering their expression on adhesion in HeLa cells. Decreasing the expression of siat7e using short interfering RNA (siRNA) resulted in greater aggregation (i.e. clumping) and morphological changes as compared to untreated anchorage-independent HeLa cells. Similar effects were seen in anchorage-independent HeLa cells when the expression of lama4 was enhanced as compared to untreated anchorage-independent HeLa cells. Using a shear flow chamber, an attachment assay was developed; illustrating either increased expression of siat7e or decreased expression of lama4 in anchorage-dependent HeLa cells reduced cellular adhesion.

As described above, the results of this study are consistent with the roles siat7e and lama4 play in adhesion processes in vivo and indicate modifying the expression of either gene can influence adhesion in HeLa cells. Madin-Darby Canine Kidney (MDCK) cells have been previously established as hosts for the production of a number of viruses, including the avian influenza virus.

The conversion of anchorage-dependent cells to cells capable of growing in suspension will simplify the production process and represent an attractive replacement to the current production procedure in chicken embryonated eggs. In terms of large-scale virus production, MDCK cells grown in suspension conditions are more advantageous than the use of attached cell lines. MDCK cells have been reported in literature as good candidates for inactivated virus vaccine. Embryonated chicken eggs have been used for many decades as hosts for influenza virus propagation; however, continuous cell lines have several advantages over embryonated chicken eggs for inactivated virus vaccine production, including a more readily available host system, they are more robust and scalable, they allow for the production of avian strains, and the HA antigen is theoretically more similar to the native form. Described herein is the transfection of the anchorage-dependent MDCK cells with the human siat7e gene, its effect on the properties of the siat7e-expressing cells and their capability to produce the influenza virus.

From previous studies, siat7e was demonstrated to have an effect in cell adhesion. Consequently, the ability of the human siat7e gene to assist the adaptation of adherent MDCK cells into suspension was investigated. The rate of adaptation to suspension, morphological features, and viabilities of MDCK cells in the presence or absence of siat7e gene expression was compared.

The goals of the experiments are to determine the ability of genetically modified adherence-independent MDCK cell line cultivated in suspension to support replication of influenza viruses and to determine the virus yield in the suspension culture of genetically modified adherence-independent MDCK cell line in comparison with that of the parental MDCK cell line grown in monolayer. In the experiments, two variants of the MDCK cells are used:(1) genetically modified adherence-independent MDCK cell line cultivated in suspension, and (2) parental MDCK cell line grown in monolayer. The model influenza virus is inoculated into the growth media of each cell lines at three different doses (multiplicity of infection, m.o.i. [ID50/cell]=1.0; 0.1; and 0.01). The accumulation of the progeny virus is tested at sequential time points post infection by determination of virus titer (hemagglutination, HA, and infectivity, ID50). As a control the separate flask of each cell line is cultivated without virus inoculation for the whole time period (8 days). Before infection both cell lines were maintained at 37 C in an atmosphere of 5% CO₂. After virus inoculation, the temperature is maintained at 33 C (optimal for virus replication). Table 1 shows a sheet for sample collection.

TABLE 1

| | |
|---|---|
| M.o.i., | Sample number |

| ID₅₀/cell | 2 days p.i. | 4 days p.i. | 6 days p.i. | 8 days p.i. |
|---|---|---|---|---|
| 1.0 | 1 | 2 | 3 | 4 |
| 0.1 | 5 | 6 | 7 | 8 |
| 0.01 | — | 9 | 10 | 11 |
| Control | | | | 12 |

### Example 2

### Transfection of MDCK Cells with Human siat7e and its Effects on Cell-Cell Adhesion and Cell Spreading

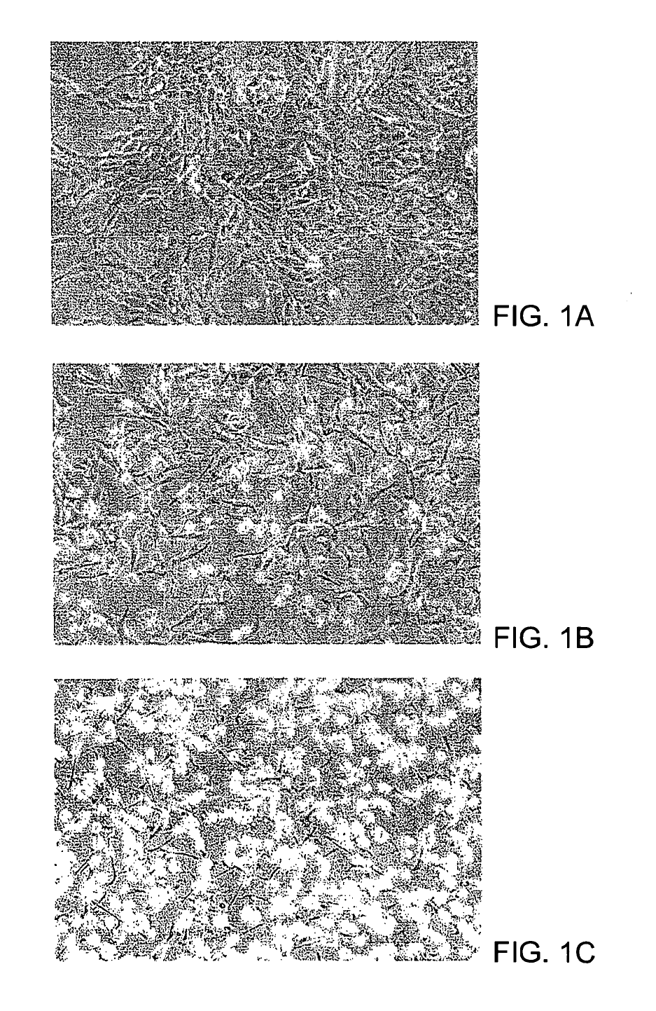

Anchorage-dependent MDCK cells exhibited changes in cell-cell adhesion and cell spreading behavior following the incorporation of the human siat7e gene, shown in FIG. 1. Cells transfected with the siat7e shown in FIG. 1B (clone 1) and FIG. 1C (clone 2) appear to spread less on the cell culture flask than the parental cells shown in FIG. 1A; the siat7e-expressing cells also lost their ability to form a tight junctions with the neighboring cells. It was also observed that when the siat7e-expressing cells undergo prolonged culture, some cells self-detach while maintaining their viability.

Assessment of transfection efficiencies with the siat7e plasmid using the FACSCalibur machine showed that approximately 4% of MDCK cells were transfected 24 hours after introducing the plasmid vector.

### Example 3

### Gene Expression Differences Between the Parental and the siat7e-Expressing MDCK Cells

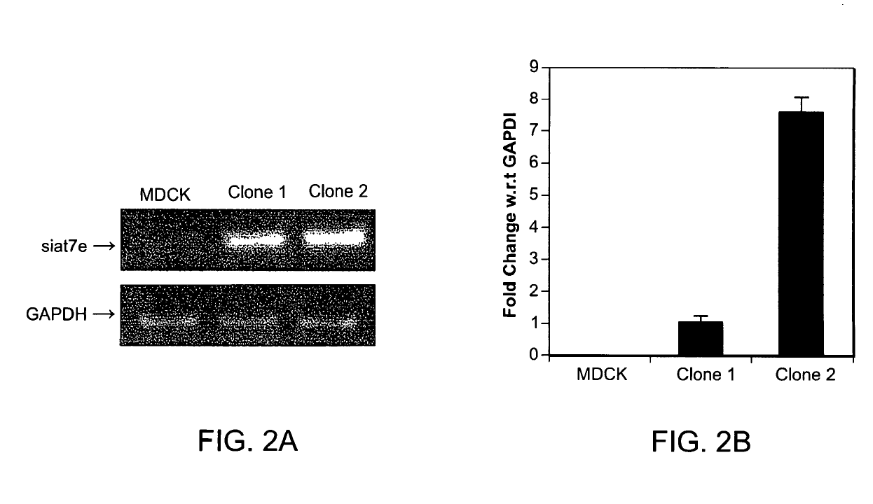

The detection of the siat7e mRNA in the parental and the siat7e-expressing cells and the expression of the housekeeping gene (endogenous GAPDH) are shown in FIG. 2A. Expression of siat7e can be seen in the transfected cells but no expression can be seen in the parental cells, while GADPH expression was detected in all samples. Real-time PCR was performed to quantify the expression of siat7e and the expression of the housekeeping gene in clones 1 and 2 (FIG. 2B). It was observed that the increase in the siat7e expression was correlated with the degree of cell-cell adhesion and cell spreading of these two transfected clones seen in FIG. 1.

The cells grew well in suspension in shake flasks. The cultures reached a concentration of 7×10⁵ cells/ml maintaining above 90% viability throughout the growth. It is interesting that the canine homolog of the human siat7e gene was not identified in the parental MDCK cells and that the human gene was successfully incorporated and transcribed, (FIG. 2A) modifying considerably the cell phenotype.

### Example 4

### Surface Charge Differences Between the Parental and the siat7e-Expressing MDCK Cells

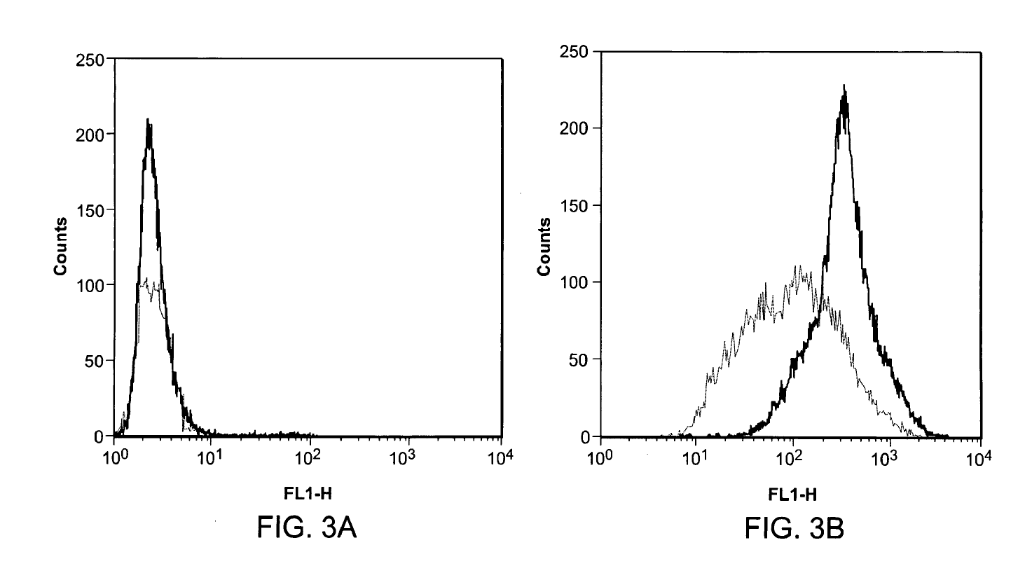

To assess cell surface difference between the two cell lines, the cell surface charge was measured using FITC-labeled cationized ferritin (24-26). The signal profiles from each cell line, with and without ferritin treatment, are shown in FIG. 3. Flow cytometric analysis showed a shift in the overall signal distribution of the siat7e-expressing cells (FIG. 3B). The shift indicates higher signal intensities emitted from the fluorescein (FITC) which corresponds to higher number of anionic sites on the membrane surface. No difference was observed when the ferritin was not present.

Thus, by using CF-FITC it was possible to determine that there is a change in the net charge on the surface of the siat7e-expressing cells. The increased negative charge might be associated with the increased number of sialic acids moieties attached to the cell surface gangliosides by siat7e. Elevated negative charge of the cell surface may contribute to a decreased cell-to-surface adhesion and to electrostatic repulsion between cells, and thus allowing the cells to grow in suspension.

### Example 5

### Growth Kinetics in Monolayer and Suspension of the Parental and siat7e-Expressing MDCK Cells

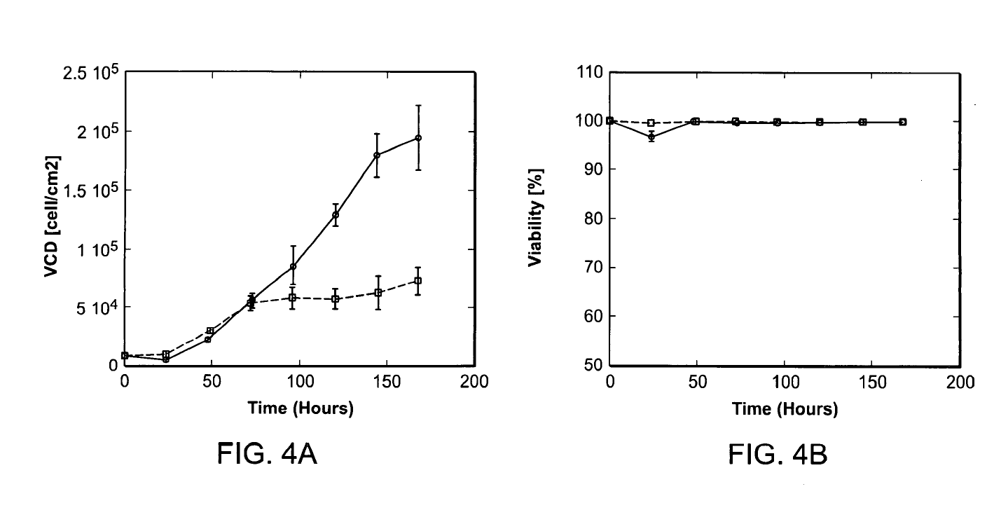

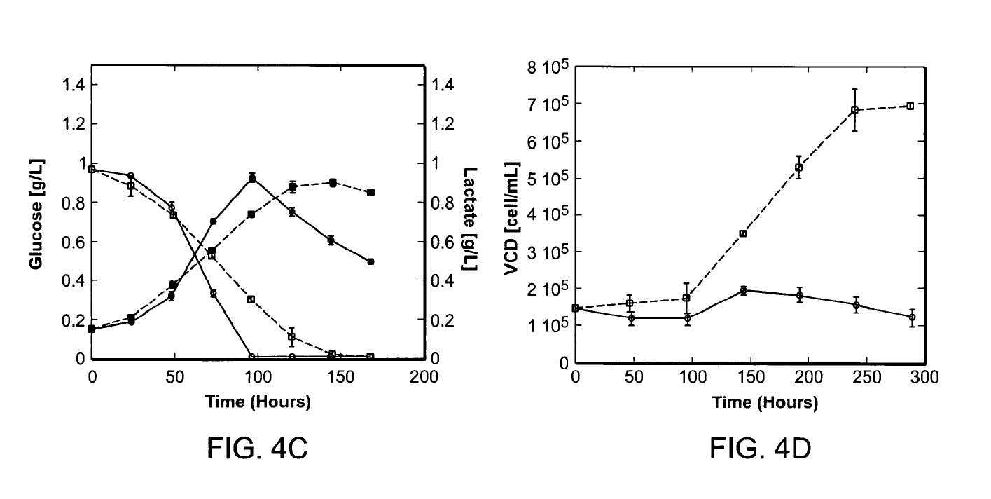

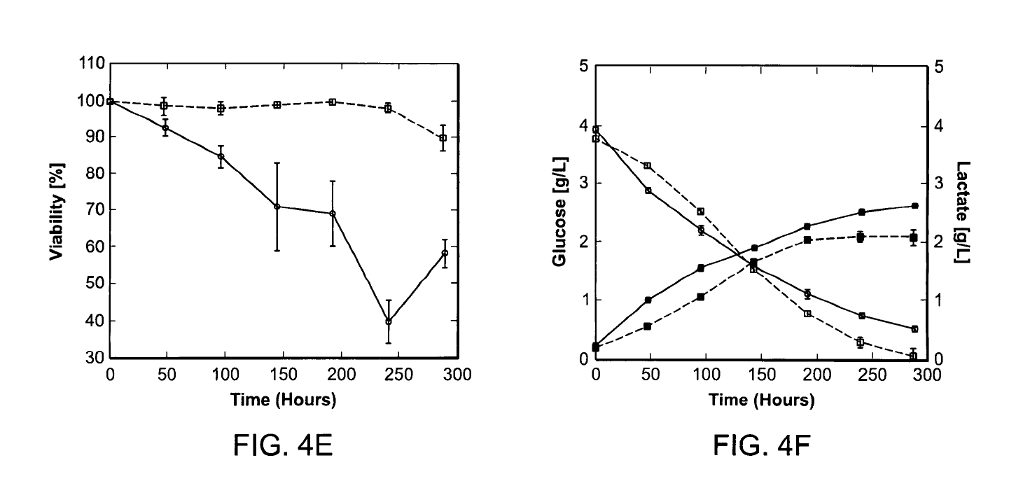

Growth, viability, glucose consumption and lactate production of the parental and the siat7e-expressing MDCK cells grown as a monolayer in T flasks are shown in FIG. 4 A-C, and grown as suspension culture in FIG. 4 D-F. The siat7e-expressing cells grew less than the parental cells in the T flask (FIG. 4A). Their density reached 7×10⁴ cells/cm² compared to 2×10⁵ cells/cm² of the parental cells after 179 hours of growth, although the percent viability of the cells was similar. Glucose consumption and lactate production in the two cell lines were similar until the siat7e-expressing cells approached peak density in the T flasks as shown in FIG. 4C. Opposite growth trends were observed when the two types of cells were propagated in shake flasks. The growth curve (FIG. 4D) demonstrates that siat7e-expressing cells were able to proliferate in suspension culture, whereas the parental cells could not. The siat7e-expressing cells grew exponentially to a concentration of 7×10⁵ cells/mL. High viabilities (FIG. 4E) of the siat7e-expressing cells were seen throughout the 12-day growth. These cells were at least 90 percent viable, while the viability of the parental MDCK cells decreased steadily. Metabolite profile shown in FIG. 4F demonstrates that parental MDCK cells were consuming glucose and producing lactate at a slightly higher rate than the siat7e-expressing cells. Microscopic analysis at the end of the growth showed that the surviving parental MDCK cells were aggregated in large clumps, while the siat7e-expressing cells appeared healthy.

### Example 6

### Influenza Virus Growth and HA Titer in Parental and siat7e-Expressing MDCK Cells

The yield of influenza virus in parental and siat7e-expressing MDCK cells was evaluated by analysis of growth kinetics of a model virus B/Victoria/504/2000 related to the constant number of cells (10⁶ cells). Table 2, shown below, shows virus titers in different cell substrates. Table 2 summarizes the highest values of both the viral and the HA titers.

TABLE 2

| | Viral Titer | | Virus Titer per 10⁶ cells | |
|---|---|---|---|---|
| | | EID₅₀/mL, | | EID₅₀, |
| Substrate | HAU/mL | log₁₀ | HAU | log₁₀ |
| MDCK | 1,810 | 8.35 ± 0.17 | 2,155 | 8.42 |
| monolayer | | | | |
| siat7e-expressing | 5,120 | 6.90 ± 0.12 | 8,606 | 7.12 |
| cells monolayer | | | | |
| siat7e-expressing | 40,960 | 7.87 ± 0.12 | 54,348 | 8.00 |
| cells^{c} suspension | | | | |

^{a}Influenza strain B/Victoria/504/2000 was used to infect the substrates between M.O.I.s of 1.0 and 2.0 TCID^{50.}
^{b}Hemagglutinin titers and infectious titers were measured using supernatant from whole cell lysate samples.
^{c}Cells were infected at 10⁷/mL density in suspension culture and then diluted to 10⁸/mL for propagation.

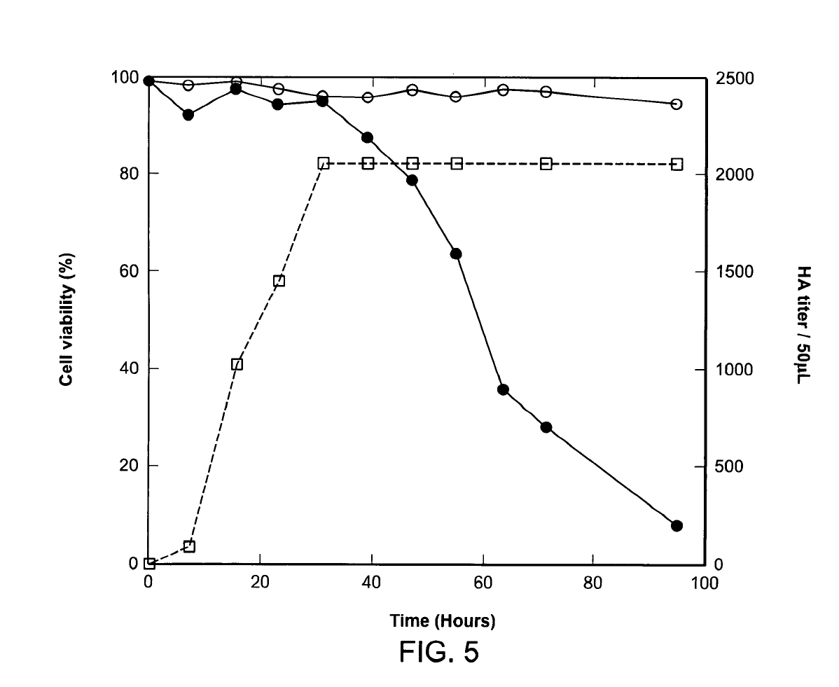

The values shown in Table 2 were obtained 36 to 48 hours post infection in the case of the adherent cells and 24-38 hours in the case of cells grown in suspension. The viral infectivity titers were similar in three growth conditions: monolayer culture of the anchorage-dependent parental MDCK cells, monolayer culture of the siat7e-expressing cells and the siat7e-expressing cells grown in suspension. However, considerable differences were observed for HA titers, expressed in hemagglutinating units (HAU). When calculated per 10⁶ cell, 2,155 HAU was obtained from the parental MDCK cells, 8,606 HAU from the siat7e-expressing cells grown in monolayer, and 54,348 HAU from the siat7e-expressing cells grown in suspension in shake flasks. FIG. 5 shows the cell viability of the infected siat7e-expressing cells grown in suspension and the HA titers over the time course of one representative kinetic experiment.

### Example 7

### Virus Antigenic Stability During Replication in Parental MDCK Cells and siat7e-Expressing Cells

The effect of different cell substrates on virus antigenic properties was evaluated in hemagglutination inhibition test (HA1). The HA1 titers of three ferret sera that were infected with egg-grown reference virus B/Victoria/504/2000 were determined using the B/Victoria output virus from the parental MDCK cells and the siat7e-expressing MDCK cells grown either in monolayers or in suspension. The results are shown in Table 3, below. Table 3 shows HA1 titers with viruses from different cells.

TABLE 3

| | HAI titers | | |
|---|---|---|---|
| Substrate | Sera No. 1 | Sera No. 2 | Sera No. 3 |
| Chicken Eggs | 128^{b} | 256 | 256 |
| siat7e-expressing | 256 | 512 | 512 |
| cells^{c} suspension | | | |
| siat7e-expressing | 64 | 128 | 128 |
| cells monolayer | | | |
| MDCK | 64 | 128 | 128 |
| monolayer | | | |

^{a}Sera were obtained from three ferrets 3 weeks after intranasal infection with egg-derived reference virus B/Victoria/504/2000.
^{b}Reciprocal of the highest dilution of serum capable of completely inhibiting HA activity of the respective virus. Data from a single representative experiment.
^{c}Cells were infected at 10^{τ}/mL density in suspension culture and then diluted to 10⁸/mL for propagation.

In all cases, the sera titers were within two-fold difference, demonstrating that cell-derived viruses were as antigenic as those obtained from the egg-derived reference virus. Direct DNA sequencing of RT-PCR products amplified from HA and NA (neuraminidase) viral gene segments, showed that the cell-derived viruses and the egg-derived reference virus had identical nucleotide sequences. These data demonstrate that replication of the virus in parental or siat7e-expressing cells did not alter the antigenic properties of the virus.

### Example 8

### Wave Bioreactor Growth

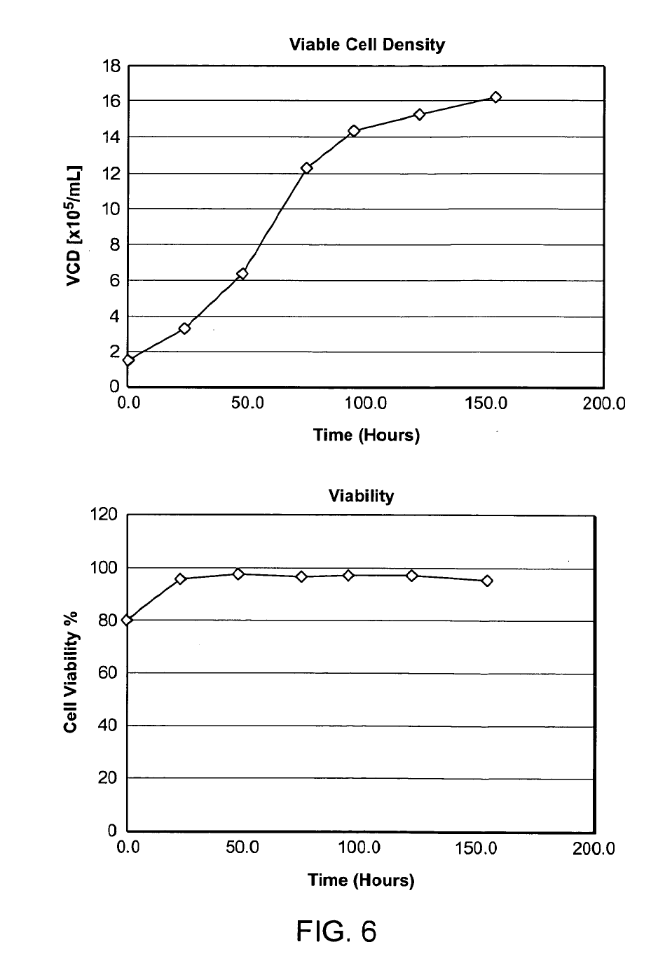

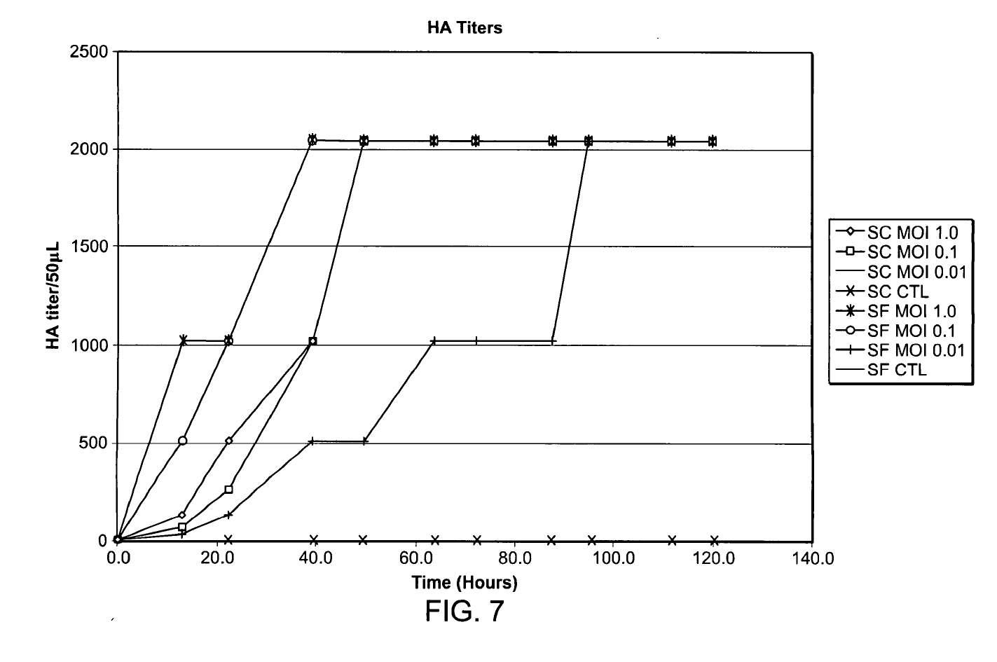

The growth of siat7e-expressing MDCK cells in suspension in bioreactors was investigated next. Table 4, shown below, details growth of MDCK_siat7e clone 2 p. 21 in a bioreactor. The Table lists the growth medium that was used, and the percent viability of the cells taken at the times indicated. VCD is viable cell density. FIG. 6 is two graphs that show the results of these experiments.

TABLE 4

| Hours | VCD | Viab % |
|---|---|---|
| 0.0 | 1.53 | 79.9 |
| 23.3 | 3.31 | 95.8 |
| 47.8 | 6.41 | 97.8 |
| 74.8 | 12.34 | 96.9 |
| 94.7 | 14.45 | 97.5 |
| 122.0 | 15.33 | 97.5 |
| 154.3 | 16.28 | 95.9 |

Previously, two genes were identified that have a role in cell adhesion (1): siat7e, a type II membrane glycosylating sialytransferase, and lama 4 which encodes laminin α4, a member of the laminin family of glycoproteins. These two genes were identified following a comparison of gene transcription of two phenotypically distinct HeLa cells, anchorage-dependent and anchorage-independent. It was demonstrated that decreased expression of siat7e in the anchorage-independent HeLa cells, or enhanced expression of lama4, resulted in greater aggregation and morphological changes compared with the untreated anchorage-independent HeLa cells. An opposite effect was observed when expression of siat7e was increased and lama4 expression was decreased in the anchorage-dependent HeLa cells.

Influenza virus is currently being produced in embryonated eggs (27). Since the production in eggs is quite cumbersome and time consuming, replacing the embryonated eggs process with mammalian cells, is an area that is currently being investigated. (5, 7, 9). However, because MDCK cells are anchorage-dependent, replacement of the embryonated eggs with these cells would still present a difficulty in production. Conversion of these cells to grow in suspension would simplify and shorten the production process.

Incorporation of the human gene siat7e into the MDCK cells, as shown herein, resulted in their conversion to anchorage-independent cells. Siat7e-expressing cells were not only able to grow in suspension and to produce identical virus to the one produced in embryonated eggs, their specific production of HA was about 20 times higher than the anchorage-dependent parental cells.

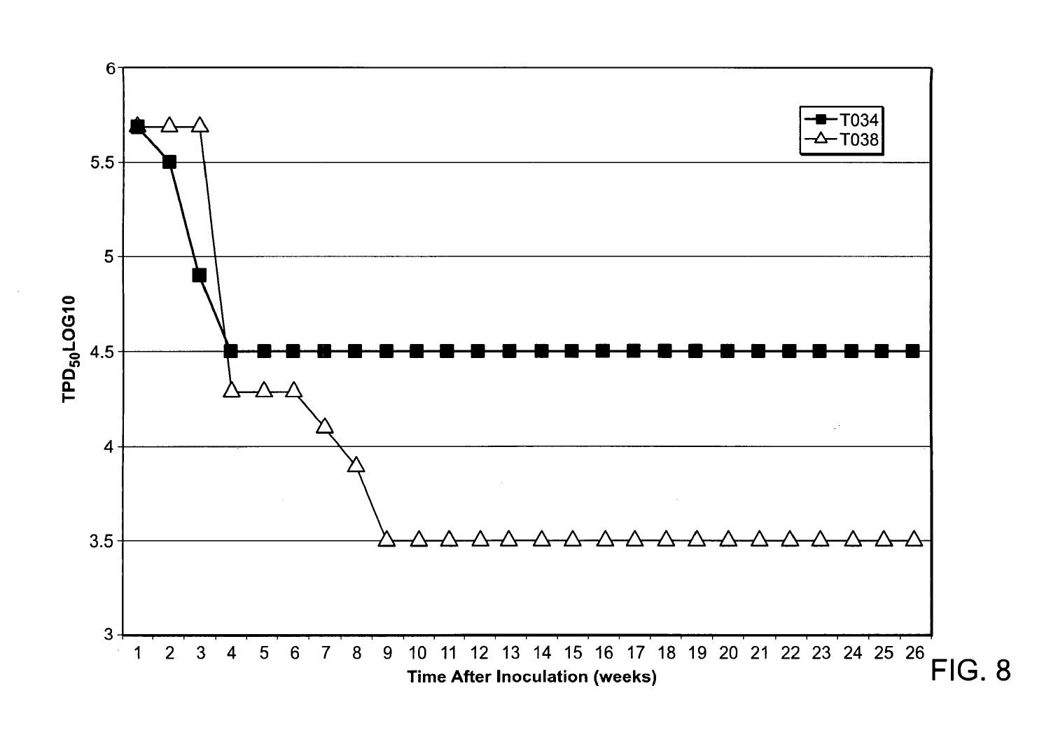

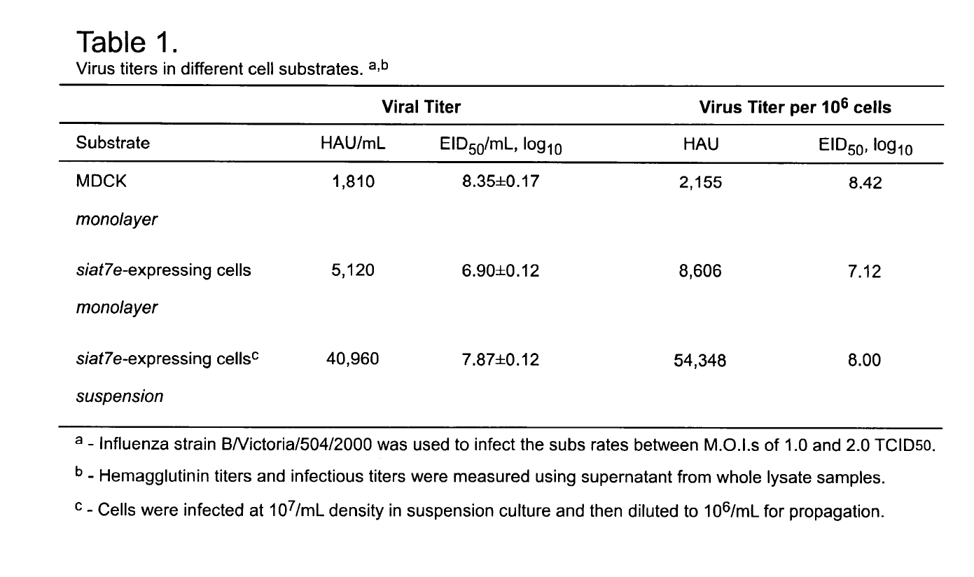

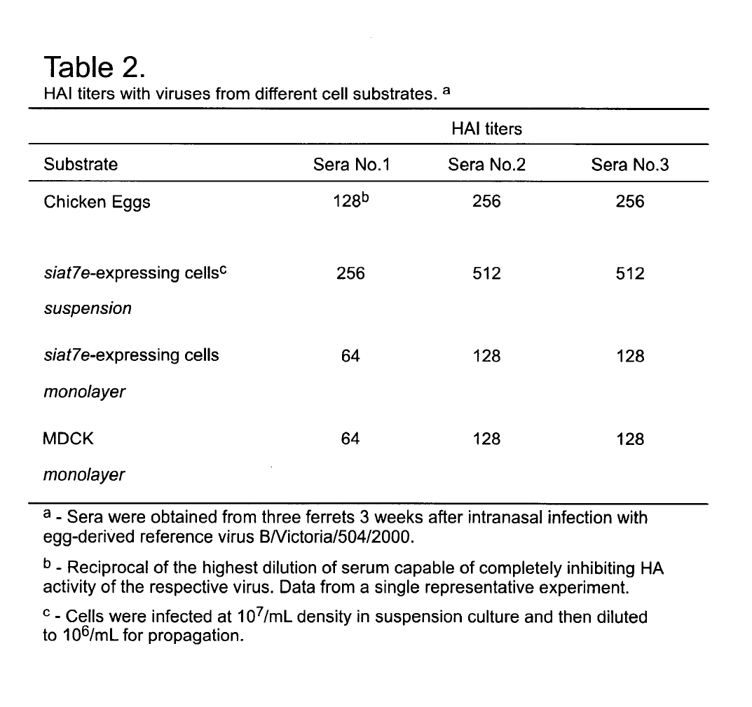

The tumorigenicity of the parental (T038) and the siat7e-expressing (T034) MDCK cells was also examined. FIG. 8 shows the tumorigenicity analysis, where the results are expressed in tumor producing dose at the 50% end point (TPD₅₀), i.e. the number of cells required for tumor formation, TPD₅₀ Log 10 over a period of 26 weeks. Results were generated from 5 nude mice at each dosage level. These results show that tumorigenicity did not vary considerably between the two cell lines.

### Methods

The Examples described were performed using, but not limited to, the following materials and methods.

### Cell Line and Virus

Madin Darby Canine Kidney (MDCK) cells were acquired from American Type Culture Collection (Manassas, Va.) (Cat. No. CCL-34). The MDCK cells were grown in 37° C., 5% CO₂ humid incubator using Minimal Essential Medium containing Earl's salts and L-glutamine (Invitrogen, Carlsbad, Calif.) and supplemented with Fetal Bovine Serum (Invitrogen) to a final concentration of 10%. Only cells growing in less than 20 passages were used for this study. Influenza virus strain B/Victoria/504/2000 was obtained from the influenza virus depository of the Center of Biologics Evaluations and Research, Food and Drugs Administration (Bethesda, Md.).

### Establishment of Stable MDCK Cell Line Expressing Siat7e

Escherichia coli DH5a competent cells (Invitrogen) were transformed with full-length human siat7e gene expression vector (Cat. No. EX-V1581-M03, Genecopoeia, Germantown, Md.). The plasmids were purified using the QIAprep Spin Miniprep kit (Qiagen, Germantown, Md.) and were used to transfect MDCK cells using Lipofectamine 2000 reagent under manufacturer's protocol (Invitrogen). The transfected procedure was as follows: day 1: MDCK cells were seeded at 2×10⁵ cells/well in a 24-well plate; day 2: 0.8 μg of plasmid DNA was mixed with 2.0 μL of Lipofectamine 2000 and incubated together with the cells in OptiMEM I medium (Invitrogen) for 4 hours; the cells were than washed and suspended in growth medium; day 3: G418 was added to the growth medium at a final concentration of 0.400 mg/mL, and the medium containing G418 (selective medium) was routinely replaced every 3 to 4 days for a period of 3 weeks. Stably transfected pool of siat7e-expressing cells were grown and banked. Finally, clones were isolated by limiting dilution in a 96-well plate.

### Gene Expression

RNA samples were isolated from parental MDCK cells and from clones of the siat7e-expressing cells using RNeasy Total RNA Isolation kit (Qiagen). Superscript One-Step RT-PCR kit (Invitrogen) was used for the reverse transcription and for PCR amplification experiments in accordance to the manufacturer's protocol, using the sense primer sequence 5′-TTACTCGCCACAAGATGCTG-3′ and antisense primer sequence 5′-GCACCATGCCATAAACATTG-3′. GAPDH was selected as the endogenous control gene and was amplified using sense primer sequence 5′-AACATCATCCCTGCTTCCAC-3′ and antisense primer sequence 5′-GACCACCTGGTCCTCAGTGT-3′. Briefly: cDNA synthesis was performed at 50° C. for 30 min, samples were incubated at 94° C. for 2 min to “hot-start” the DNA Taq polymerase. The PCR amplification cycle consisted of denaturation at 94° C. for 15 sec annealing at 55° C. for 30 sec, and extending at 72° C. for 10 sec (14 sec for the endogenous control). The target genes were amplified for 35 cycles with a final extension at 72° C. for 10 min. The end products were resolved on a 1% agarose gel at 130V for 30 minutes and captured on the gel imager (BioRad, Hercules, Calif.).

Real-time PCR was performed using Power SYBR® Green RNA-to-C_{T}™1-Step Kit (Applied Biosystems, Foster City, Calif.) with the same primer sequences described above. Briefly: cDNA samples were synthesized from 0.5 ng RNA sample and amplified under standard thermal cycler protocol (50° C. for 2 min, 95° C. for 10 min, and 40 cycles of 95° C. for 15 s and 60° C. for 1 min). Target Ct values were averaged from replicates and fold changes were calculated against the endogenous control, GAPDH.

### Cationized Ferritin Binding Assay

Cationized ferrtin (Electron Microscopy Sciences, Hatfield, Pa.) was conjugated with FITC using the FITC Protein Labeling kit (Pierce Biotechnology, Rockford, Ill.). Briefly: cationized ferritin was dialyzed with the supplied borate buffer and incubated with FITC solution at room temperature for 1 hour. Excess FITC dye was removed using a dialysis cassette (Pierce Biotechnology). Conjugated ferritin complex was quantified using E270 nm^{1%}=79.9 and MW=750,000 for native ferritin and a correction factor of 0.3 for FITC whose λmax=494 nm. The calculated F/P ratio was approximately 12. Approximately 1×10⁷ cells were detached from culture flasks using Hank's-based cell dissociation buffer (Invitrogen) and washed with PBS before resuspending in 1 mL PBS containing FITC-conjugated ferritin at 50 μg/mL final concentration (24-26). The mixture was incubated on a thermomixer at 4° C. for 1 hour and washed once with PBS. Cells were spun down and suspended in 1 mL cold PBS. The cells were immediately analyzed using the FACSCalibur flow cytometer.

### Growth Kinetics

For growth kinetics in anchorage-dependent manner, parental and siat7e-expressing MDCK cells were seeded at a concentration of 2×10⁵ cells per one 25 cm² culture flask; 21 flasks were seeded for each cell line. Glucose and lactate concentrations were measured using the YSI 2700 Select biochemistry analyzer (YSI Life Sciences, Yellow Springs, Ohio) and cell count was measured using Cedex (Innovatis AG, Bielefeld, Germany). Measurements were taken daily from 3 flasks. For growth kinetics in suspension culture, cells from each line were seeded at approximately 2×10⁵ cell/mL in three 125 mL vented shake flasks containing 30 mL of serum-supplemented Dulbecco's Modified Eagle's Medium (Invitrogen) and shaken at 90 RPM. Measurements were taken at 48 hours intervals.

### Virus Growth Evaluations in Monolayer and Suspension Culture

Monolayer culture: Parental MDCK cells or siat7e-expressing cells were grown to confluency in 25 cm² flasks (Corning, USA). After removal of the growth media, the cells were washed once with serum-free medium and the virus was added to each flask at a multiplicity of infection (MOI) of 2.0 TCID₅₀ (50%-tissue culture infectious dose). After adsorption for 1 hour at 37° C., the cells were washed with serum-free medium, and 10 ml of growth medium (containing 10% FBS) were added. The infected cells were incubated at 33° C. for the remainder of the experiment. Cell condition (appearance of cytopathogenic effect) was constantly monitored and samples were collected every 8 hours for virus infectivity and hemagglutination (HA) titers determination.

Suspension culture: siat7e-expressing cells grown in shake flasks were concentrated by centrifugation (600 rcf for 5 minutes) and resuspended in a serum-free medium at a density of 10⁷ cells/ml. After infection with the influenza virus at an MOI of 2.0 TCID₅₀, the cell suspension was incubated at constant shaking at 37° C. for 1 hour. At this time, the cells were precipitated and suspended in DMEM supplemented with 10% FBS to a density of 10⁶ cells/ml. The infected cells were incubated at 33° C. in the same conditions for the remainder of the experiment; the controlled culture was treated in the same way but without addition of the virus. Samples were taken every 8 hours during a period of 4 days and stored in aliquots at −70° C. for virus infectivity titer and HA titer determination. Cell concentration, viability and metabolic parameters were monitored at each time point.

### Determination of Virus Yield

Virus growth and concentration were determined by infectivity titer in chicken embryonated eggs (EID₅₀) and by HA titer using standard techniques described earlier (33-35).

### Determination of Virus Stability During Replication in MDCK Cells

Antigenic properties of the progeny virus harvested from the parental or the siat7 e-expressing cells (56 hours post infection) were characterized by hemagglutination inhibition test (HA1 test) using a set of three homologous ferret antisera specific to strain B/Victoria/504/2000. The HA1 test was performed in 96-well plates (two replicates for each serum sample) using 0.5% chicken red blood cells in PBS (pH 7.2) (35). Two viruses were considered antigenically indistinguishable if the corresponding HA1 titers did not exceed two-fold difference. In addition the nucleotide sequences of viral gene segments encoding viral surface glycoproteins, HA and NA, were determined by direct DNA-sequencing of the RT-PCR products and compared with those of the parental virus stock.

### Other Embodiments

From the foregoing description, it will be apparent that variations and modifications may be made to the invention described herein to adopt it to various usages and conditions. Such embodiments are also within the scope of the following claims.

All patents and publications mentioned in this specification are herein incorporated by reference to the same extent as if each independent patent and publication was specifically and individually indicated to be incorporated by reference.

### REFERENCES

1. Jaluria, P., Betenbaugh, M., Konstantopoulos, K., Frank, B. & Shiloach, J. (2007) Application of microarrays to identify and characterize genes involved in attachment dependence in HeLa cells. Metab Eng 9, 241-251.
2. Simonsen, L., Fukuda, K., Schonberger, L. B. & Cox, N. J. (2000) The impact of influenza epidemics on hospitalizations. J Infect Dis 181, 831-837.
3. Bardiya, N. & Bae, J. H. (2005) Influenza vaccines: recent advances in production technologies. Appl Microbiol Biotechnol 67, 299-305.
4. Kistner, O. et al. (1998) Development of a mammalian cell (Vero) derived candidate influenza virus vaccine. Vaccine 16, 960-968.
5. Govorkova, E. A., Kodihalli, S., Alymova, I. V., Fanget, B. & Webster, R. G. (1999) Growth and immunogenicity of influenza viruses cultivated in Vero or MDCK cells and in embryonated chicken eggs. Dev Biol Stand 98, 39-51; discussion 73-34.
6. Pau, M. G. et al. (2001) The human cell line PER.C6 provides a new manufacturing system for the production of influenza vaccines. Vaccine 19, 2716-2721.
7. Tree, J. A., Richardson, C., Fooks, A. R., Clegg, J. C. & Looby, D. (2001) Comparison of large-scale mammalian cell culture systems with egg culture for the production of influenza virus A vaccine strains. Vaccine 19, 3444-3450.
8. Ozaki, H. et al. (2004) Generation of high-yielding influenza A viruses in African green monkey kidney (Vero) cells by reverse genetics. J Virol 78, 1851-1857.
9. Youil, R. et al. (2004) Comparative study of influenza virus replication in Vero and MDCK cell lines. J Virol Methods 120, 23-31.
10. Frisch, S. M. & Francis, H. (1994) Disruption of epithelial cell-matrix interactions induces apoptosis. J Cell Biol 124, 619-626.
11. Folkman, J. & Moscona, A. (1978) Role of cell shape in growth control. Nature 273, 345-349.
12. Guadagno, T. M., Ohtsubo, M., Roberts, J. M. & Assoian, R. K. (1993) A link between cyclin A expression and adhesion-dependent cell cycle progression. Science 262, 1572-1575.
13. Hansen, L. K., Mooney, D. J., Vacanti, J. P. & Ingber, D. E. (1994) Integrin binding and cell spreading on extracellular matrix act at different points in the cell cycle to promote hepatocyte growth. Mol Biol Cell 5, 967-975.
14. Zhu, X., Ohtsubo, M., Bohmer, R. M., Roberts, J. M. & Assoian, R. K. (1996) Adhesion-dependent cell cycle progression linked to the expression of cyclin D1, activation of cyclin E-cdk2, and phosphorylation of the retinoblastoma protein. J Cell Biol 133, 391-403
15. Fang, F., Orend, G., Watanabe, N., Hunter, T. & Ruoslahti, E. (1996) Dependence of cyclin E-CDK2 kinase activity on cell anchorage. Science 271, 499-502.
16. Assoian, R. K. (1997) Anchorage-dependent cell cycle progression. J Cell Biol 136, 1-4.
17. Genzel, Y., Behrendt, I., Konig, S., Sann, H. & Reichl, U. (2004) Metabolism of MDCK cells during cell growth and influenza virus production in large-scale microcarrier culture. Vaccine 22, 2202-2208.
18. Genzel, Y., Fischer, M. & Reichl, U. (2006) Serum-free influenza virus production avoiding washing steps and medium exchange in large-scale microcarrier culture. Vaccine 24, 3261-3272.
19. Genzel, Y., Olmer, R. M., Schafer, B. & Reichl, U. (2006) Wave microcarrier cultivation of MDCK cells for influenza virus production in serum containing and serum-free media. Vaccine 24, 6074-6087.
20. Hu, A. Y. et al. (2008) Microcarrier-based MDCK cell culture system for the production of influenza H5N1 vaccines. Vaccine.
21. Tsuchida, A. et al. (2003) Synthesis of disialyl Lewis a (Le(a)) structure in colon cancer cell lines by a sialyltransferase, ST6GalNAc VI, responsible for the synthesis of alpha-series gangliosides. J Biol Chem 278, 22787-22794.
22. Hakomori, S. I. (2000) Cell adhesion/recognition and signal transduction through glycosphingolipid microdomain. Glycoconj J 17, 143-151.
23. Regina Todeschini, A. & Hakomori, S. I. (2008) Functional role of glycosphingolipids and gangliosides in control of cell adhesion, motility, and growth, through glycosynaptic microdomains. Biochim Biophys Acta 1780, 421-433.
24. Argueso, P., Tisdale, A., Spurr-Michaud, S., Sumiyoshi, M. & Gipson, I. K. (2006) Mucin characteristics of human corneal-limbal epithelial cells that exclude the rose bengal anionic dye. Invest Ophthalmol V is Sci 47, 113-119.
25. Danon, D., Goldstein, L., Marikovsky, Y. & Skutelsky, E. (1972) Use of cationized ferritin as a label of negative charges on cell surfaces. J Ultrastruct Res 38, 500-510.
26. King, C. A. & Preston, T. M. (1977) Fluoresceinated cationised ferritin as a membrane probe for anionic sites at the cell surface. FEBS Lett 73, 59-63.
27. Palese, P. Making better influenza virus vaccines? (2006) Emerg Infect Dis 12, 61-65.
28. Hatakeyama, S. et al. (2005) Enhanced expression of an alpha-2,6-linked sialic acid on MDCK cells improves isolation of human influenza viruses and evaluation of their sensitivity to a neuraminidase inhibitor. J Clin Microbiol 43, 4139-4146.
29. Matrosovich, M., Matrosovich, T., Carr, J., Roberts, N. A. & Klenk, H. D. (2003) Overexpression of the alpha-2,6-sialyltransferase in MDCK cells increases influenza virus sensitivity to neuraminidase inhibitors. J Virol 77, 8418-8425.
30. Oh, D. Y., Barr, I. G., Mosse, J. A. & Laurie, K. L. (2008) MDCK-SIAT1 cells show improved isolation rates for recent human influenza viruses compared to conventional MDCK cells. J Clin Microbiol 46, 2189-2194.
31. Vodeiko, G. M., McInnis, J., Chizhikov, V. & Levandowski, R. A. (2003) Genetic and phenotypic analysis of reassortants of high growth and low growth strains of influenza B virus. Vaccine 21, 3867-3874.
32. Lugovtsev, V. Y., Vodeiko, G. M., Strupczewski, C. M., Ye, Z. & Levandowski, R. A. (2007) Generation of the influenza B viruses with improved growth phenotype by substitution of specific amino acids of hemagglutinin. Virology 365, 315-323.
33. Lugovtsev, V. Y., Vodeiko, G. M. & Levandowski, R. A. (2005) Mutational pattern of influenza B viruses adapted to high growth replication in embryonated eggs. Virus Res 109, 149-157.
34. Palmer, D. F., Coleman, M. T., Dowdle, W. R., Schild, G. C. Advanced laboratory techniques for influenza diagnosis, Vol. 6. (Washington, D.C.; 1975).
35. WHO. (2002) WHO manual on animal influenza diagnosis and surveillance.

1. A method of producing an immunogenic composition comprising a virus, the method comprising:
isolating a virus from a virus infected cell, the cell comprising an expression vector comprising a nucleic acid molecule encoding a polypeptide selected from the group consisting of: siat7e, lama4, cdk13, cox15, egr1, gas6, map3k9, and gap43;
thereby producing an immunogenic composition comprising a virus.

2. A method of producing a virus comprising a polynucleotide encoding a recombinant polypeptide, the method comprising:
isolating a virus from a virus infected cell, the cell comprising an expression vector comprising a nucleic acid molecule encoding a polypeptide selected from the group consisting of: siat7e, lama4, cdk13, cox15, egr1, gas6, map3k9, and gap43,
thereby producing a virus comprising a polynucleotide encoding a recombinant polypeptide.

3. A method of producing a virus comprising a polynucleotide encoding a recombinant polypeptide, the method comprising:
isolating a virus from a virus infected cell, the cell comprising an expression vector comprising a nucleic acid molecule encoding a polypeptide corresponding to a sialyltransferase,
thereby producing a virus comprising a polynucleotide encoding a recombinant polypeptide.

4. A method of producing an virus comprising a polynucleotide encoding a recombinant polypeptide, the method comprising:
isolating a virus from a virus infected cell, the cell comprising an expression vector comprising a nucleic acid molecule encoding a polypeptide corresponding to a laminin glycoprotein,
thereby producing a virus comprising a polynucleotide encoding a recombinant polypeptide.

5. A method of producing an immunogenic composition comprising a virus, the method comprising:
isolating a virus from a virus infected cell, the cell comprising an expression vector comprising a nucleic acid molecule encoding a polypeptide corresponding to a sialyltransferase;
thereby producing an immunogenic composition comprising a virus.

6-13. (canceled)

14. A virus produced according to the method of claim 2.

15. A method of producing a vaccine or an immunogenic composition in a cell comprising:
infecting a cell comprising an expression vector comprising a nucleic acid molecule encoding a sialyltransferase polypeptide with a virus;
producing virus in the cell; and
harvesting the virus;
thereby producing a vaccine in the cell, or
A method of producing a vaccine or an immunogenic composition in a cell comprising:
infecting a cell comprising an expression vector comprising a nucleic acid molecule encoding a laminin glycoprotein with a virus;
producing virus in the cell; and
harvesting the virus;
thereby producing a vaccine in the cell, or,
A method of producing a vaccine or an immunogenic composition in a cell comprising:
infecting a cell comprising an expression vector comprising a nucleic acid molecule encoding a sialyltransferase polypeptide with a virus;
producing virus in the cell; and
harvesting the virus;
thereby producing an immunogenic composition in the cell, or,
A method of producing a vaccine or an immunogenic composition in a cell comprising:
infecting a cell comprising an expression vector comprising a nucleic acid molecule encoding a laminin glycoprotein with a virus;
producing virus in the cell; and
harvesting the virus;
thereby producing an immunogenic composition in the cell, or
A method of producing a vaccine or an immunogenic composition in a cell comprising:
infecting a cell, wherein the cell comprises a mutation that alters the expression or activity of a sialyltransferase polypeptide with a virus;
producing virus in the cell; and
harvesting the virus;
thereby producing a virus or an immunogenic composition in the cell, or
A method of producing a vaccine or an immunogenic composition in a cell comprising:
infecting a cell, wherein the cell comprises a mutation that alters the expression or activity of a laminin glycoprotein with a virus;
producing virus in the cell; and
harvesting the virus;
thereby producing a virus or an immunogenic composition in the cell, or
A method of producing a vaccine or an immunogenic composition in a cell comprising:
infecting a cell comprising an expression vector comprising a nucleic acid molecule encoding a polypeptide selected from the group consisting of: siat7e, lama4, cdk13, cox15, egr1, gas6, map3k9, and gap43 with a virus;
producing virus in the cell; and
harvesting the virus;
thereby producing a vaccine in the cell, or
A method of producing a vaccine or an immunogenic composition in a cell comprising:
infecting a cell comprising an expression vector comprising a nucleic acid molecule encoding a siat7e, lama4, cdk13, cox15, egr1, or gas6 inhibitory nucleic acid molecule with a virus;
producing virus in the cell; and
harvesting the virus;
thereby producing an immunogenic composition in the cell, or
A method of producing a vaccine or an immunogenic composition in a cell comprising:
infecting a cell, wherein the cell comprises a mutation that alters the expression or activity of a polypeptide selected from the group consisting of siat7e, lama4, cdk13, cox15, egr1, gas6, map3k9, and gap43 polypeptide with a virus;
producing virus in the cell; and
harvesting the virus;
thereby producing a virus or an immunogenic composition in the cell, or

16-45. (canceled)

46. An immunogenic composition produced by the method of claim 1 in a pharmaceutically acceptable carrier.

47. (canceled)

48. A vaccine produced by the method of claim 1.

49-52. (canceled)

53. A virus produced by the method of claim 1 in a pharmaceutically acceptable carrier.

54-56. (canceled)

57. A method of producing an immune response in a subject comprising:
administering to the subject the pharmaceutical composition of claim 46 in an amount sufficient to generate an immune response,
thereby producing an immune response in a subject.

58. A method of treating a subject suffering from a viral infection comprising:
administering to the subject the pharmaceutical composition of claim 46 in an amount sufficient to generate an immune response,
thereby treating a subject suffering from a viral infection.

59. A method of preventing a viral infection in a subject comprising:
administering to the subject the pharmaceutical composition of claim 46 in an amount sufficient to generate an immune response,
thereby preventing a viral infection in a subject.

60-70. (canceled)

71. The method of claim 57, wherein the pharmaceutical composition is administered in multiple doses over an extended period of time.

72-74. (canceled)

75. A method of polynucleotide therapy in a subject comprising:
identifying a gene product to be expressed;
preparing a composition according to claim 71, wherein the virus is an adenovirus or adeno-associated virus that expresses a coding sequence that codes for the gene product; and
administering the composition to a subject.

76-79. (canceled)

80. A cell comprising an expression vector comprising a nucleic acid molecule encoding a polypeptide selected from the group consisting of: siat7e, lama4, cdk13, cox15, egr1, gas6, map3k9, and gap43, and a virus, or
A cell comprising an expression vector comprising a nucleic acid molecule encoding a sialyltransferase inhibitory nucleic acid molecule, and a virus, or
A cell comprising an expression vector comprising a nucleic acid molecule encoding a laminin glycoprotein inhibitory nucleic acid molecule, and a virus, or
A cell comprising an expression vector comprising a nucleic acid molecule encoding a siat7e, lama4, cdk13, cox15, egr1, or gas6 inhibitory nucleic acid molecule, and a virus, or
A cell comprising a mutation that alters the expression or activity of a polypeptide selected from the group consisting of cdk13, siat7e, lama4, cox15, egr1, gas6, map3k9, and gap43 polypeptide, and a virus.

81-117. (canceled)

118. A method of producing a vaccine or immunogenic composition, the method comprising isolating a virus from the cell of claim 80, and incorporating an effective amount of the virus into a pharmaceutically acceptable excipient.

119-150. (canceled)

151. A kit comprising the immunogenic composition of claim 46 and instructions for use.

152. A kit comprising the vaccine of claim 48 and instructions for use.

153. A kit comprising the virus of claim 53 and instructions for use.

154-155. (canceled)


SEQUENCE LISTING (20 sequences)

SEQ ID NO: 1 - DNA, 1632 bp, Homo sapiens
ggaactacgcagagccagaccagcgggaccacagaatgggctgaggcggcggcggctgtttggataaagtcaacagcgggacgtggggcgtgacgccgtagtaaaagcccagcttgaaaatggagatgtatgaaacccttggaaaagtgggagagggaagttacggaacagtcatgaaatgtaaacataagaatactgggcagatagtggccattaagatattttatgagagaccagaacaatctgtcaacaaaattgcgatgagagaaataaagtttctaaagcaatttcatcacgaaaacctggtcaatctgattgaagtttttagacagaaaaagaaaattcatttggtatttgaatttattgaccacacagtattagatgagttacaacattattgtcatggactagagagtaagcgacttagaaaatacctcttccagatccttcgagcaattgactatcttcacagtaataatatcattcatcgagatataaaacctgagaatattttagtatcccagtcaggaattactaagctctgtgattttggttttgcacgaacactagcagctcctggggacatttatacggactatgtggccacacgctggtatagagctcccgaattagtattaaaagatacttcttatggaaaacctgtggatatctgggctttgggctgtatgatcattgagatggccactggaaatccctatcttcctagtagttctgatttggatttactccataaaattgttttgaaagtgggcaatttgtcacctcacttgcagaatatcttttccaagagccccatttttgctggggtagttcttcctcaagttcaacaccccaaaaatgcaagaaaaaaatatccaaagcttaatggattgttggcagatatagttcatgcttgtttacaaattgatcctgctgacaggatatcatctagtgatcttttgcatcatgagtattttactagagatggatttattgaaaaattcatgccagaactgaaagctaaattactgcaggaagcaaaagtcaattcattaataaagccaaaagagagttctaaagaaaatgaactcaggaaagatgaaagaaaaacagtttataccaatacactgctaagtagttcagttttgggaaaggaaatagaaaaagagaaaaagcccaaggagatcaaagtcagagttattaaagtcaaaggaggaagaggagatatctcagaaccaaaaaagaaagagtatgaaggtggacttggtcaacaggatgcaaatgaaaatgttcatcctatgtctccagatacaaaacttgtaaccattgaaccaccaaaccctatcaatcccagcactaactgtaatggcttgaaagaaaatccacattgcggaggttctgtgacaatgccacccatcaatctaactaacagtaatttgatggctgcaaatctcagttcaaatctctttcaccccagtgtgaggtgagctgtaacagagaagaaacctaaataatacaacattcctgtataatggtatttcaaagaatcgtgttcatagtgtctgtatgtaaactgaacttgaagaaaatatattgaaattaaagctgtataatgggccaaaaaaaaaaaaa

SEQ ID NO: 2 - protein, 455 aa, Homo sapiens
MEMYETLGKVGEGSYGTVMKCKHKNTGQIVAIKIFYERPEQSVNKIAMREIKFLKQFHHENLVNLIEVFRQKKKIHLVFEFIDHTVLDELQHYCHGLESKRLRKYLFQILRAIDYLHSNNIIHRDIKPENILVSQSGITKLCDFGFARTLAAPGDIYTDYVATRWYRAPELVLKDTSYGKPVDIWALGCMIIEMATGNPYLPSSSDLDLLHKIVLKVGNLSPHLQNIFSKSPIFAGVVLPQVQHPKNARKKYPKLNGLLADIVHACLQIDPADRISSSDLLHHEYFTRDGFIEKFMPELKAKLLQEAKVNSLIKPKESSKENELRKDERKTVYTNTLLSSSVLGKEIEKEKKPKEIKVRVIKVKGGRGDISEPKKKEYEGGLGQQDANENVHPMSPDTKLVTIEPPNPINPSTNCNGLKENPHCGGSVTMPPINLTNSNLMAANLSSNLFHPSVR

SEQ ID NO: 3 - DNA, 2048 bp, Homo sapiens
ctctgcaacagccgcgcttcccgggtcccgcggctcccgcgcgcgatctgccgcggccggctgctgggcaaaaatcagagccgcctccgccccattacccatcatggaaaccctccaggaaaaagtggccccggacgcgcgagcctgaggattctgcacaaaagaggtgcccaaaatgaagaccctgatgcgccatggtctggcagtgtgtttagcgctcaccaccatgtgcaccagcttgttgctagtgtacagcagcctcggcggccagaaggagcggcccccgcagcagcagcagcagcagcagcaacagcagcagcaggcgtcggccaccggcagctcgcagccggcggcggagagcagcacccagcagcgccccggggtccccgcgggaccgcggccactggacggatacctcggagtggcggaccacaagcccctgaaaatgcactgcagggactgtgccctggtgaccagctcagggcatctgctgcacagtcggcaaggctcccagattgaccagacagagtgtgtcatccgcatgaatgacgcccccacacgcggctatgggcgtgacgtgggcaatcgcaccagcctgagggtcatcgcgcattccagcatccagaggatcctccgcaaccgccatgacctgctcaacgtgagccagggcaccgtgttcatcttctggggccccagcagctacatgcggcgggacggcaagggccaggtctacaacaacctgcatctcctgagccaggtgctgccccggctgaaggccttcatgattactcgccacaagatgctgcagtttgatgagctcttcaagcaggagactggcaaagacaggaagatatccaacacttggctcagcactggctggtttacaatgacaattgcactggagctctgtgacaggatcaatgtttatggcatggtgcccccagacttctgcagggatcccaatcacccttcagtaccttatcattattatgaaccttttggacctgatgaatgtacaatgtacctctcccatgagcgaggacgcaagggcagtcatcaccgctttatcacagagaaacgagtctttaagaactgggcacggacattcaatattcacttttttcaaccagactggaaaccagaatcacttgctataaatcatcctgagaataaacctgtgttctaaggaatgagcatgccagactgtaatcccaggtattcactgcatcagacaccgagacactgaacttcctgagccaccagacaggaaagggtagcagaaaacagcttcactcctcaggaagtaccatggacagacgcctaccaggggtgacaaagcagtgcagttggattgtaaggaaaaattccggaattaatgcatcctaatgaatgttgtccccttcaatggtgttaccttaggagctgaacattcaattcagttacaccactatgactaaaaacagtttggatctcttagtattgcctttgaaactgcaacataagcaactcaacaatattagttgcattcctttatagacataccatgtcaaagacgtttttctatcaagttgtattctttcctgttctataacctttgtcatctgttagactctgtatgtgtgatttgtaaaaagcaggctgaaactatggacatgatttctgaagagcacatctccactgactttcataaagcaaatgtccaatatttatttattgagagttttttagtgcaatctgggccagtatttttatagattatgattatgtggtaatttatccttcctaactctttaatcctgaatgatggttggaaatggcctagaattaggttactctgttcacaatgctcattgttagcatgcaattggtatttgacttggaagtgttgtgttgtattttttgaacccctaggcttcaggaaaactgctcttttgtaaaaagaatagcgatgacattttctaatgtgcagaaatgttccaaaaggacaaaattgaaaaccaaaaactatgttattaaaacaaaaaaatgctaaaaaaaaaaaaaaaaaa

SEQ ID NO: 4 - protein, 336 aa, Homo sapiens
MKTLMRHGLAVCLALTTMCTSLLLVYSSLGGQKERPPQQQQQQQQQQQQASATGSSQPAAESSTQQRPGVPAGPRPLDGYLGVADHKPLKMHCRDCALVTSSGHLLHSRQGSQIDQTECVIRMNDAPTRGYGRDVGNRTSLRVIAHSSIQRILRNRHDLLNVSQGTVFIFWGPSSYMRRDGKGQVYNNLHLLSQVLPRLKAFMITRHKMLQFDELFKQETGKDRKISNTWLSTGWFTMTIALELCDRINVYGMVPPDFCRDPNHPSVPYHYYEPFGPDECTMYLSHERGRKGSHHRFITEKRVFKNWARTFNIHFFQPDWKPESLAINHPENKPVF

SEQ ID NO: 5 - DNA, 7287 bp, Homo sapiens
agcttagagtgggagggcctgggagtagaaggtaaaaagggagtggtgagaatgaatgtgagaaggaagccaggacagcgcagtccccagtcccgaacggccagggagaggaggtggcctagcgctggcggggctcaccccaatccgtctgccttttgatgccgtactctgctggttgcgcagccacctcgggatactgcacacggagaggagggaaaataagcgaggcaccgccgcaccacgcgggagacctacggagacccacagcgcccgagccctggaagagcactactggatgtcagcggagaaatggctttgagctcagcctggcgctcggttctgcctctgtggctcctctggagcgctgcctgctcccgcgccgcgtccggggacgacaacgcttttccttttgacattgaagggagctcagcggttggcaggcaagacccgcctgagacgagcgaaccccgcgtggctctgggacgcctgccgcctgcggccgagaaatgcaatgctggattctttcacaccctgtcgggagaatgtgtgccctgcgactgtaatggcaattccaacgagtgtttggacggctcaggatactgtgtgcactgccagcggaacacaacaggagagcactgtgaaaagtgtctggatggttatatcggagattccatcaggggagcaccccaattctgccagccgtgcccctgtcccctgccccacttggccaattttgcagaatcctgctataggaaaaatggagctgttcggtgcatttgtaacgaaaattatgctggacctaactgtgaaagatgtgctcccggttactatggaaaccccttactcattggaagcacctgtaagaaatgtgactgcagtggaaattcagatcccaacctgatctttgaagattgtgatgaagtcactggccagtgtaggaattgcttacgcaacaccaccggattcaagtgtgaacgttgcgctcctggctactatggggacgccaggatagccaagaactgtgcagtgtgcaactgcgggggaggcccatgtgacagtgtaaccggagaatgcttggaagaaggttttgaaccccctacaggcatggactgcccaaccataagctgtgataagtgcgtctgggacctgactgatgcactgcggttagcagcgctctccatcgaggaaggcaaatccggggtgctgagcgtatcctctggggccgccgctcataggcacgtgaatgaaatcaacgccaccatctacctcctcaaaacaaaattgtcagaaagagaaaaccaatacgccctaagaaagatacaaatcaacaatgctgagaacacgatgaaaagccttctgtctgacgtagaggaattagttgaaaaggaaaatcaagcctccagaaaaggacaacttgttcagaaggaaagcatggacaccattaaccacgcaagtcagctggtagagcaagcccatgatatgagggataaaatccaagagatcaacaacaagatgctctattatggggaagagcatgaacttagccccaaggaaatctctgagaagctggtgttggcccagaagatgcttgaagagattagaagccgtcaaccatttttcacccaacgggagctcgtggatgaggaggcagatgaggcttacgaactactgagccaggctgagagctggcagcggctgcacaatgagacccgcactctgtttcctgtcgtcctggagcagctggatgactacaatgctaagttgtcagatctccaggaagcacttgaccaggcccttaactatgtcagggatgccgaagacatgaacagggccacagcagccaggcagcgggaccatgagaaacaacaggaaagagtgagggaacaaatggaagtggtgaacatgtctctgagcacatctgcggactctctgacaacacctcgtctaactctttcagaacttgatgatataataaagaatgcgtcagggatttatgcagaaatagatggagccaaaagtgaactacaagtaaaactatctaacctaagtaacctcagccatgatttagtccaagaagctattgaccatgcacaggaccttcaacaagaagctaatgaattgagcaggaagttgcacagttcagatatgaacgggctggtacagaaggctttggatgcatcaaatgtctatgaaaatattgttaattatgttagtgaagccaatgaaacagcagaatttgctttgaacaccactgaccgaatttatgatgcggtgagtgggattgatactcaaatcatttaccataaagatgaaagtgagaacctcctcaatcaagccagagaactgcaagcaaaggcagagtctagcagtgatgaagcagtggctgacactagcaggcgtgtgggtggagccctagcaaggaaaagtgcccttaaaaccagactcagtgatgccgttaagcaactacaagcagcagagagaggggatgcccagcagcgcctggggcagtctagactgatcaccgaggaagccaacaggacgacgatggaggtgcagcaggccactgcccccatggccaacaatctaaccaactggtcacagaatcttcaacattttgactcttctgcttacaacactgcagtgaactctgctagggatgcagtaagaaatctgaccgaggttgtccctcagctcctggatcagcttcgtacggttgagcagaagcgacctgcaagcaacgtttctgccagcatccagaggatccgagagctcattgctcagaccagaagtgttgccagcaagatccaagtctccatgatgtttgatggccagtcagctgtggaagtgcactcgagaaccagtatggatgacttaaaggccttcacgtctctgagcctgtacatgaaaccccctgtgaagcggccggaactgaccgagactgcagatcagtttatcctgtacctcggaagcaaaaacgccaaaaaagagtatatgggtcttgcaatcaaaaatgataatctggtatacgtctataatttgggaactaaagatgtggagattcccctggactccaagcccgtcagttcctggcctgcttacttcagcattgtcaagattgaaagggtgggaaaacatggaaaggtgtttttaacagtcccgagtctaagtagcacagcagaggaaaagttcattaaaaagggggaattttcgggagatgactctctgctggacctggaccctgaggacacagtgttttatgttggtggagtgccttccaacttcaagctccctaccagcttaaacctgcctggctttgttggctgcctggaactggccactttgaataatgatgtgatcagcttgtacaactttaagcacatctataatatggacccctccacatcagtgccatgtgcccgagataagctggccttcactcagagtcgggctgccagttacttcttcgatggctccggttatgccgtggtgagagacatcacaaggagagggaaatttggtcaggtgactcgctttgacatagaagttcgaacaccagctgacaacggccttattctcctgatggtcaatggaagtatgtttttcagactggaaatgcgcaatggttacctacatgtgttctatgattttggattcagcggtggccctgtgcatcttgaagatacgttaaagaaagctcaaattaatgatgcaaaataccatgagatctcaatcatttaccacaatgataagaaaatgatcttggtagttgacagaaggcatgtcaagagcatggataatgaaaagatgaaaataccttttacagatatatacattggaggagctcctccagaaatcttacaatccagggccctcagagcacaccttcccctagatatcaacttcagaggatgcatgaagggcttccagttccaaaagaaggacttcaatttactggagcagacagaaaccctgggagttggttatggatgcccagaagactcacttatatctcgcagagcatatttcaatggacagagcttcattgcttcaattcagaaaatatctttctttgatggctttgaaggaggttttaatttccgaacattacaaccaaatgggttactattctattatgcttcagggtcagacgtgttctccatctcactggataatggtactgtcatcatggatgtaaagggaatcaaagttcagtcagtagataagcagtacaatgatgggctgtcccacttcgtcattagctctgtctcacccacaagatatgaactgatagtagataaaagcagagttgggagtaagaatcctaccaaagggaaaatagaacagacacaagcaagtgaaaagaagttttacttcggtggctcaccaatcagtgctcagtatgctaatttcactggctgcataagtaatgcctactttaccagggtggatagagatgtggaggttgaagatttccaacggtatactgaaaaggtccacacttctctttatgagtgtcccattgagtcttcaccattgtttctcctccataaaaaaggaaaaaatttatccaagcctaaagcaagtcagaataaaaagggagggaaaagtaaagatgcaccttcatgggatcctgttgctctgaaactcccagagcggaatactccaagaaactctcattgccacctttccaacagccctagagcaatagagcacgcctatcaatatggaggaacagccaacagccgccaagagtttgaacacttaaaaggagattttggtgccaaatctcagttttccattcgtctgagaactcgttcctcccatggcatgatcttctatgtctcagatcaagaagagaatgacttcatgactctatttttggcccatggccgcttggtttacatgtttaatgttggtcacaaaaaactgaagattagaagccaggagaaatacaatgatggcctgtggcatgatgtgatatttattcgagaaaggagcagtggccgactggtaattgatggtctccgagtcctagaagaaagtcttcctcctactgaagctacctggaaaatcaagggtcccatttatttgggaggtgtggctcctggaaaggctgtgaaaaatgttcagattaactccatctacagttttagtggctgtctcagcaatctccagctcaatggggcctccatcacctctgcttctcagacattcagtgtgaccccttgctttgaaggccccatggaaacaggaacttacttttcaacagaaggaggatacgtggttctagatgaatctttcaatattggattgaagtttgaaattgcatttgaagtccgtcccagaagcagttccggaaccctggtccacggccacagtgtcaatggggagtacctaaatgttcacatgaaaaatggacaggtcatagtgaaagtcaataatggcatcagagatttttccacctcagttacacccaagcagagtctctgtgatggcagatggcacagaattacagttattagagattctaatgtggttcagttggatgtggactctgaagtgaaccatgtggttggacccctgaatccaaaaccaattgatcacagggagcctgtgtttgttggaggtgttccagaatctctactgacaccacgcttggcccccagcaaacccttcacaggctgcatacgccactttgtgattgatggacacccagtgagcttcagtaaagcagccctggtcagcggcgccgtaagcatcaactcctgtccagcagcctgacatgacagagcacagctgcccaaatacaaagttctttagagcactgaaagaaacacaaagccagccaggaggaacagtaactcttccttcgggtggaagctttcatcgagttgaacaggacttaaacgaatcatcagggaccggatatttcttatttctcatttggattcttaaccttgaatccaaagtgtctgcaatggacaacaattgaaggagtggcaaacttacttgtattgagagcacacgcaattcctactggtgaaattactgtttctgtttctaataaaatagaagggattccaaataaacacttgcacacatttttgaagtgcggctagattctcagattcacctttcttccagggaagataactttcaatctatataaaaatctctgtcctaaaactacctttctttattttgaagagacttactaacttacatataatctaaattagatgatagatttgtttttagcccttttgtttggtctatcagtataagaagaatattttaggtttatagctgaagttatcaaggtttaataaagtaaatttctaacagaatactagaaaaatgcagtataatttaattttttctaaataagaaacacaggaaatcaactactttttccccttccttatctccttaaaagaaaaataaaattgtacatgagaggaggcttctgtaggttattattaccattattgtgtgttctatgggaatcattgaggatatcacagcaaaaacagtaggacaaaatcataaaattcaatttaagagtacacaagtccttttattaaaagtttgctcctagcctgggcaacataatgagatcccatctctgcaaaaaaatttgtacatgggcatacacctgtagtcccagctacttgggaggctgagacgggaggatcgcttaagctcaggagttcaaggctgcagtgagctatgactgctgactgtacctgcactccagcctgggcaacagagtgagatcctgtctcaaaaacaaagtgtgctctccacatacctgcaacacaactagtcttatttctaaaatgttataatcttttttccaagtagctacattaatatagtctagaaaaaaatggacttgaatagctggtagaatattaaaatatagaaatgaaataaaagaattatatctaaaaacctcaactcagaagacagaaaaagagaaaataggccctgatatcaacagaattaacaatacataaaaggagtaacttttgaggggagaggatataaaatattttgaggaattaccaaggggaataaaacaatgttaccttgaaatgattatatatatattacatattggtatatatgtccatacctacctatatcccctgctacccttctgtctgaaatatacaaataatgataatgttgaagatatcgataaacatagctaatgtctgttcatagaggacttactaagtgccagccaccatgataagctaaagttaattattttatttgttc

SEQ ID NO: 6 - protein, 1823 aa, Homo sapiens
MALSSAWRSVLPLWLLWSAACSRAASGDDNAFPFDIEGSSAVGRQDPPETSEPRVALGRLPPAAEKCNAGFFHTLSGECVPCDCNGNSNECLDGSGYCVHCQRNTTGEHCEKCLDGYIGDSIRGAPQFCQPCPCPLPHLANFAESCYRKNGAVRCICNENYAGPNCERCAPGYYGNPLLIGSTCKKCDCSGNSDPNLIFEDCDEVTGQCRNCLRNTTGFKCERCAPGYYGDARIAKNCAVCNCGGGPCDSVTGECLEEGFEPPTGMDCPTISCDKCVWDLTDALRLAALSIEEGKSGVLSVSSGAAAHRHVNEINATIYLLKTKLSERENQYALRKIQINNAENTMKSLLSDVEELVEKENQASRKGQLVQKESMDTINHASQLVEQAHDMRDKIQEINNKMLYYGEEHELSPKEISEKLVLAQKMLEEIRSRQPFFTQRELVDEEADEAYELLSQAESWQRLHNETRTLFPVVLEQLDDYNAKLSDLQEALDQALNYVRDAEDMNRATAARQRDHEKQQERVREQMEVVNMSLSTSADSLTTPRLTLSELDDIIKNASGIYAEIDGAKSELQVKLSNLSNLSHDLVQEAIDHAQDLQQEANELSRKLHSSDMNGLVQKALDASNVYENIVNYVSEANETAEFALNTTDRIYDAVSGIDTQIIYHKDESENLLNQARELQAKAESSSDEAVADTSRRVGGALARKSALKTRLSDAVKQLQAAERGDAQQRLGQSRLITEEANRTTMEVQQATAPMANNLTNWSQNLQHFDSSAYNTAVNSARDAVRNLTEVVPQLLDQLRTVEQKRPASNVSASIQRIRELIAQTRSVASKIQVSMMFDGQSAVEVHSRTSMDDLKAFTSLSLYMKPPVKRPELTETADQFILYLGSKNAKKEYMGLAIKNDNLVYVYNLGTKDVEIPLDSKPVSSWPAYFSIVKIERVGKHGKVFLTVPSLSSTAEEKFIKKGEFSGDDSLLDLDPEDTVFYVGGVPSNFKLPTSLNLPGFVGCLELATLNNDVISLYNFKHIYNMDPSTSVPCARDKLAFTQSRAASYFFDGSGYAVVRDITRRGKFGQVTRFDIEVRTPADNGLILLMVNGSMFFRLEMRNGYLHVFYDFGFSGGPVHLEDTLKKAQINDAKYHEISIIYHNDKKMILVVDRRHVKSMDNEKMKIPFTDIYIGGAPPEILQSRALRAHLPLDINFRGCMKGFQFQKKDFNLLEQTETLGVGYGCPEDSLISRRAYFNGQSFIASIQKISFFDGFEGGFNFRTLQPNGLLFYYASGSDVFSISLDNGTVIMDVKGIKVQSVDKQYNDGLSHFVISSVSPTRYELIVDKSRVGSKNPTKGKIEQTQASEKKFYFGGSPISAQYANFTGCISNAYFTRVDRDVEVEDFQRYTEKVHTSLYECPIESSPLFLLHKKGKNLSKPKASQNKKGGKSKDAPSWDPVALKLPERNTPRNSHCHLSNSPRAIEHAYQYGGTANSRQEFEHLKGDFGAKSQFSIRLRTRSSHGMIFYVSDQEENDFMTLFLAHGRLVYMFNVGHKKLKIRSQEKYNDGLWHDVIFIRERSSGRLVIDGLRVLEESLPPTEATWKIKGPIYLGGVAPGKAVKNVQINSIYSFSGCLSNLQLNGASITSASQTFSVTPCFEGPMETGTYFSTEGGYVVLDESFNIGLKFEIAFEVRPRSSSGTLVHGHSVNGEYLNVHMKNGQVIVKVNNGIRDFSTSVTPKQSLCDGRWHRITVIRDSNVVQLDVDSEVNHVVGPLNPKPIDHREPVFVGGVPESLLTPRLAPSKPFTGCIRHFVIDGHPVSFSKAALVSGAVSINSCPAA

SEQ ID NO: 7 - DNA, 4860 bp, Homo sapiens
cacaaggtcgcagggccgttatgaggggaccccgggactcgaaccttggctccacagctgagccattctcgctacctgcccctcgtcacgccctccgtttccacacctttaacgcctcaaagatagaaggtgccgcccaaggggctggaaggagctgaggaaacgactccagaagaaatcaccactgactacgactcccgccggcccgccccggggagccttcggccgaccgtcccctcccccgccaccttccgcaccggccttcccggacggtatccgcgctcgttttcgctcagaggaggccccctgccttttcatgctccacgcgttcctccctcgtgcgcctgcagtttccacttggaatttgggctccggcgcgcaccagctaagaagcgcgtcaacagctgcgcgcgcccgtgcgcgcgtccccgacacctacgccccagcagcccccgcgaaagcggagtcgcaacgcaggcgcacttctgttcgctccggtccccagagaaggcggggctcccgctgcccgacccggaagtgcttctcttttccttggcggaggagggagaccacagagccctgggttgtggaagaggtggctgttccctgtcatcagtatgcagcgattgctctttccgccgttgagggccttgaaggggaggcagtatctgccgctcctggctcctagggcagcgcctagagcacagtgtgattgcatcaggcgccctttgaggccagggcaatacagcaccatctctgaagtagctttgcaatctggaaggggtacagtgtcccttccctcaaaggctgctgagcgggtggtgggccgatggctcctggtctgcagtggaacagtggctggagcagttattcttggtggagtaactaggttgacagagtctggcctctcgatggtagattggcatttaataaaggagatgaagccacctacaagccaagaggaatgggaagcagaattccaaagataccagcaatttccagaatttaaaatcttgaatcatgatatgacactgacagaattcaagttcatctggtacatggagtactcacaccgaatgtggggtcgccttgtaggccttgtgtacatcctgcctgctgcctacttttggagaaagggctggctcagccgtggcatgaaaggacgtgttcttgccctctgtggcctcgtctgcttccagggtctgttgggatggtatatggtgaaaagtggactagaagaaaaatcagactcccatgacatccctcgggtcagtcagtaccgccttgctgcccacctgggatcagccctggttctttattgtgccagcttgtggacctcactgtcactgctactccctccgcacaagttgcctgaaacccaccaactcctacagttgagacgatttgctcatggaacagcaggtctggtgttccttacggccctctcaggggcttttgtggcagggctagatgctgggcttgtttataactcctttcccaaaatgggagaatcctggatcccggaggacctctttaccttctcccccatcctgaggaatgtttttgagaatcccaccatggtgcagtttgatcaccggattctgggaatcacttcagtcactgccattacagtgctctacttcctctctcggagaattccccttcctagaaggaccaagatggcagcagtgactctgctggctttggcgtatacacaggtgggcttgggcatcagcacgctgctgatgtatgtcccaactcctctggccgccactcaccagtcaggctccttggctttgctcactggtgctctttggctgatgaatgaactccgaagagtcccaaaatgattcttagaggaccagcctcctgggactgtgactgcttttgagagctcttcagagatcataagaacttgggcttttctacgagatgaccttgacataccaagtggtttccaaatggtcaacttacttaaaaatcttttcctgttttgagatagtcactggatcaagaatgcattaagtgtggttaccctaaatgttcccttttaaatctgcttttcatgttgaaaatcagttttaatgtagagaaagaaatgtctgccatttgctgcttaacaggctttgtgtcaggtttttcagtgttggcaagctcttggttctacgtggatgatttctacgtggatgttctcctgagtccttaattctgcctaaatagaatttctttatgatcttaacttcacttccattaggtgaagattgaaaggataggattgacatacccaaagtagccagctggtcttcagcaaaaaaaaaaaagctaatgtcttctaatatcctgattttcagaaatggggaaattgcagattttgacaagcttaatgtgtatatgtagcaaaatggtttttaagtacttggaaaaggaggtaatcgccaaatagttccatttttttttttttttttgagacagagtttcactcttgttgcccaggctggagtacaatggcgtgatctcagctcactctgcaacctccacttcccaggttcaagcgattctcatgcctcagcctcccgagtagctgggactacaggcacaagtcaccatgcctggctaattttgtatttttagtagagaaggggtttcaccacattggccgggctggtcttgaactcctgacctcaggtaatccgcccatctcggcttcccaaagtgctgagattacaggcatgggccaccacgcccagctagttcctcttttgataaggctgttaacattacaaggtcagagagaggaatggaatcgacagtataaattggttcctgagagggtttcctaacagctttgtaataagaaaaatatggccttgatggtgtagtgcatggacatctacccagataaacaaattgttgactgctgaattaatgtgattttggtctctattgatatttcatagggccctgtcttattcaactttacttttaaaatcagtgatttggatgaaggcatcagaaacatctgatttgaggatggagcaacccaatattgctaatataatagatgacagtggcaactgaaaacagtcccagaatgaccccatcgagatgaaattaaacagattagaaatgtaacatactcatttaaattaaataagttgtataagtttgggctgaagaaacctggccttatatcatttcatataaaaaagacgggattttagttgtcctcaggttcaatatgaaccaagtagtattcatgttgttattatatgataacagaaaaattagtggtacttcaggttttatcagtagaagtgtcacagccatagtcaaaatacccctaacatactgcatttcactctgggtaccaaatttaagcaagatattgatggccactaagtgtgtatccagaagaagagatcagaatgatgagtccagaacccctgtcacacagggaataggtgaaggaactagggatatttaactggagcagagaacatctggagagagagcaagattcctgtccttgagaagttgaagccgtctcatgcagaagagggaatgagcttccattatgctgcagagctggcagcaatggatgaaagttgcaggaagttagttttagttctgtagaaggcaagagtagggtagaccaaatcaaatgctcactggggtcaggctgatggagacagtaacactggcgagcaggtgccccagctcggactatactgctgagttctggtctgttgctgccactctacaggtccagggttgttagatcttctgggtttttgttttgctttttgaaataaggaaaataagcttatcttaagtttatttttccttggatatacctagattccatacagtgacaatacaaatacaaaggaagaacatctggatttcatccttgacctcatttacaaagcaaaaagagatgctggattcagatataaatgtttaattttgcagtgtttaagtcagcaaatcttctgttttttaaaaataagccacatatctagatttttctgtgaaagctctcaattaagtgttgggaactaatttcaagattttaaaaaatgttatgcaggcttaacgtgtctgagagccataaatgacctaacgtttcccgttagtctctgaactaggtgatctcagttctctttcaactccagtactctgtaagtttctgtgacctgtagtgtaccattctcaggtggtgatatggtttggatgttttgtcccctccaaatctcatgttcaaagtgtgacattcagtgttagaggtgggcctagtaggaggtatttgggtcatgggggcggatcccttatgtatgactttgtgccatccccatagtaatgagtgagttctcactctggttgtttatgcaagagctggttgtttaaaagagcctggaacttcctcctctctcgcttgctctctctcaccatgtgacacactggctccccttcaccttctgccatgattgtaagcttcgtgaggccctcaccagaagcagatgccagcgccatgcttcctatacagttctatgcagaacagtgagccaaataaacctcttttctttctaaaaattgtcagtatttccttatagcaatgcaagccgactaacacaggtgattcttagcaaaacagtttgtcaatttttcataaatgctggaccctggctgggcttatgctattgcctagaagagattcccaacttctctcttatttgaatcttagaaaaatcccaaagcccaagcctcatctaagaccaattaagtcacaatccctggaggaagtaaaaggcatatttttaaagttccccaggtgattccaatgtgcagacaagtctaggg

SEQ ID NO: 8 - protein, 410 aa, Homo sapiens
MQRLLFPPLRALKGRQYLPLLAPRAAPRAQCDCIRRPLRPGQYSTISEVALQSGRGTVSLPSKAAERVVGRWLLVCSGTVAGAVILGGVTRLTESGLSMVDWHLIKEMKPPTSQEEWEAEFQRYQQFPEFKILNHDMTLTEFKFIWYMEYSHRMWGRLVGLVYILPAAYFWRKGWLSRGMKGRVLALCGLVCFQGLLGWYMVKSGLEEKSDSHDIPRVSQYRLAAHLGSALVLYCASLWTSLSLLLPPHKLPETHQLLQLRRFAHGTAGLVFLTALSGAFVAGLDAGLVYNSFPKMGESWIPEDLFTFSPILRNVFENPTMVQFDHRILGITSVTAITVLYFLSRRIPLPRRTKMAAVTLLALAYTQVGLGISTLLMYVPTPLAATHQSGSLALLTGALWLMNELRRVPK

SEQ ID NO: 9 - DNA, 3136 bp, Homo sapiens
gcgcagaacttggggagccgccgccgccatccgccgccgcagccagcttccgccgccgcaggaccggcccctgccccagcctccgcagccgcggcgcgtccacgcccgcccgcgcccagggcgagtcggggtcgccgcctgcacgcttctcagtgttccccgcgccccgcatgtaacccggccaggcccccgcaactgtgtcccctgcagctccagccccgggctgcacccccccgccccgacaccagctctccagcctgctcgtccaggatggccgcggccaaggccgagatgcagctgatgtccccgctgcagatctctgacccgttcggatcctttcctcactcgcccaccatggacaactaccctaagctggaggagatgatgctgctgagcaacggggctccccagttcctcggcgccgccggggccccagagggcagcggcagcaacagcagcagcagcagcagcgggggcggtggaggcggcgggggcggcagcaacagcagcagcagcagcagcaccttcaaccctcaggcggacacgggcgagcagccctacgagcacctgaccgcagagtcttttcctgacatctctctgaacaacgagaaggtgctggtggagaccagttaccccagccaaaccactcgactgccccccatcacctatactggccgcttttccctggagcctgcacccaacagtggcaacaccttgtggcccgagcccctcttcagcttggtcagtggcctagtgagcatgaccaacccaccggcctcctcgtcctcagcaccatctccagcggcctcctccgcctccgcctcccagagcccacccctgagctgcgcagtgccatccaacgacagcagtcccatttactcagcggcacccaccttccccacgccgaacactgacattttccctgagccacaaagccaggccttcccgggctcggcagggacagcgctccagtacccgcctcctgcctaccctgccgccaagggtggcttccaggttcccatgatccccgactacctgtttccacagcagcagggggatctgggcctgggcaccccagaccagaagcccttccagggcctggagagccgcacccagcagccttcgctaacccctctgtctactattaaggcctttgccactcagtcgggctcccaggacctgaaggccctcaataccagctaccagtcccagctcatcaaacccagccgcatgcgcaagtaccccaaccggcccagcaagacgcccccccacgaacgcccttacgcttgcccagtggagtcctgtgatcgccgcttctcccgctccgacgagctcacccgccacatccgcatccacacaggccagaagcccttccagtgccgcatctgcatgcgcaacttcagccgcagcgaccacctcaccacccacatccgcacccacacaggcgaaaagcccttcgcctgcgacatctgtggaagaaagtttgccaggagcgatgaacgcaagaggcataccaagatccacttgcggcagaaggacaagaaagcagacaaaagtgttgtggcctcttcggccacctcctctctctcttcctacccgtccccggttgctacctcttacccgtccccggttactacctcttatccatccccggccaccacctcatacccatcccctgtgcccacctccttctcctctcccggctcctcgacctacccatcccctgtgcacagtggcttcccctccccgtcggtggccaccacgtactcctctgttccccctgctttcccggcccaggtcagcagcttcccttcctcagctgtcaccaactccttcagcgcctccacagggctttcggacatgacagcaaccttttctcccaggacaattgaaatttgctaaagggaaaggggaaagaaagggaaaagggagaaaaagaaacacaagagacttaaaggacaggaggaggagatggccataggagaggagggttcctcttaggtcagatggaggttctcagagccaagtcctccctctctactggagtggaaggtctattggccaacaatcctttctgcccacttccccttccccaattactattccctttgacttcagctgcctgaaacagccatgtccaagttcttcacctctatccaaagaacttgatttgcatggattttggataaatcatttcagtatcatctccatcatatgcctgaccccttgctcccttcaatgctagaaaatcgagttggcaaaatggggtttgggcccctcagagccctgccctgcacccttgtacagtgtctgtgccatggatttcgtttttcttggggtactcttgatgtgaagataatttgcatattctattgtattatttggagttaggtcctcacttgggggaaaaaaaaaaaagaaaagccaagcaaaccaatggtgatcctctattttgtgatgatgctgtgacaataagtttgaacctttttttttgaaacagcagtcccagtattctcagagcatgtgtcagagtgttgttccgttaacctttttgtaaatactgcttgaccgtactctcacatgtggcaaaatatggtttggtttttctttttttttttttttgaaagtgttttttcttcgtccttttggtttaaaaagtttcacgtcttggtgccttttgtgtgatgcgccttgctgatggcttgacatgtgcaattgtgagggacatgctcacctctagccttaaggggggcagggagtgatgatttgggggaggctttgggagcaaaataaggaagagggctgagctgagcttcggttctccagaatgtaagaaaacaaaatctaaaacaaaatctgaactctcaaaagtctatttttttaactgaaaatgtaaatttataaatatattcaggagttggaatgttgtagttacctactgagtaggcggcgatttttgtatgttatgaacatgcagttcattattttgtggttctattttactttgtacttgtgtttgcttaaacaaagtgactgtttggcttataaacacattgaatgcgctttattgcccatgggatatgtggtgtatatccttccaaaaaattaaaacgaaaataaagtagctgcgattggg

SEQ ID NO: 10 - protein, 543 aa, Homo sapiens
MAAAKAEMQLMSPLQISDPFGSFPHSPTMDNYPKLEEMMLLSNGAPQFLGAAGAPEGSGSNSSSSSSGGGGGGGGGSNSSSSSSTFNPQADTGEQPYEHLTAESFPDISLNNEKVLVETSYPSQTTRLPPITYTGRFSLEPAPNSGNTLWPEPLFSLVSGLVSMTNPPASSSSAPSPAASSASASQSPPLSCAVPSNDSSPIYSAAPTFPTPNTDIFPEPQSQAFPGSAGTALQYPPPAYPAAKGGFQVPMIPDYLFPQQQGDLGLGTPDQKPFQGLESRTQQPSLTPLSTIKAFATQSGSQDLKALNTSYQSQLIKPSRMRKYPNRPSKTPPHERPYACPVESCDRRFSRSDELTRHIRIHTGQKPFQCRICMRNFSRSDHLTTHIRTHTGEKPFACDICGRKFARSDERKRHTKIHLRQKDKKADKSVVASSATSSLSSYPSPVATSYPSPVTTSYPSPATTSYPSPVPTSFSSPGSSTYPSPVHSGFPSPSVATTYSSVPPAFPAQVSSFPSSAVTNSFSASTGLSDMTATFSPRTIEIC

SEQ ID NO: 11 - DNA, 2521 bp, Homo sapiens
ccgagcgcttgaggtgccgcagccgccgccgccgccgccgccgcgatgtgaccttcagggccgccaggacgggatgaccggagcctccgccccgcggcgcccgcggctcgcctcggcctcccgggcgctctgaccgcgcgtccccggcccgccatggccccttcgctctcgcccgggcccgccgccctgcgccgcgcgccgcagctgctgctgctgctgctggccgcggagtgcgcgcttgccgcgctgttgccggcgcgcgaggccacgcagttcctgcggcccaggcagcgccgcgcctttcaggtcttcgaggaggccaagcagggccacctggagagggagtgcgtggaggagctgtgcagccgcgaggaggcgcgggaggtgttcgagaacgaccccgagacggattatttttacccaagatacttagactgcatcaacaagtatgggtctccgtacaccaaaaactcaggcttcgccacctgcgtgcaaaacctgcctgaccagtgcacgcccaacccctgcgataggaaggggacccaagcctgccaggacctcatgggcaacttcttctgcctgtgtaaagctggctgggggggccggctctgcgacaaagatgtcaacgaatgcagccaggagaacgggggctgcctccagatctgccacaacaagccgggtagcttccactgttcctgccacagcggcttcgagctctcctctgatggcaggacctgccaagacatagacgagtgcgcagactcggaggcctgcggggaggcgcgctgcaagaacctgcccggctcctactcctgcctctgtgacgagggctttgcgtacagctcccaggagaaggcttgccgagatgtggacgagtgtctgcagggccgctgtgagcaggtctgcgtgaactccccagggagctacacctgccactgtgacgggcgtgggggcctcaagctgtcccaggacatggacacctgtgaggacatcttgccgtgcgtgcccttcagcgtggccaagagtgtgaagtccttgtacctgggccggatgttcagtgggacccccgtgatccgactgcgcttcaagaggctgcagcccaccaggctggtagctgagtttgacttccggacctttgaccccgagggcatcctcctctttgccggaggccaccaggacagcacctggatcgtgctggccctgagagccggccggctggagctgcagctgcgctacaacggtgtcggccgtgtcaccagcagcggcccggtcatcaaccatggcatgtggcagacaatctctgttgaggagctggcgcggaatctggtcatcaaggtcaacagggatgctgtcatgaaaatcgcggtggccggggacttgttccaaccggagcgaggactgtatcatctgaacctgaccgtgggaggtattcccttccatgagaaggacctcgtgcagcctataaaccctcgtctggatggctgcatgaggagctggaactggctgaacggagaagacaccaccatccaggaaacggtgaaagtgaacacgaggatgcagtgcttctcggtgacggagagaggctctttctaccccgggagcggcttcgccttctacagcctggactacatgcggacccctctggacgtcgggactgaatcaacctgggaagtagaagtcgtggctcacatccgcccagccgcagacacaggcgtgctgtttgcgctctgggcccccgacctccgtgccgtgcctctctctgtggcactggtagactatcactccacgaagaaactcaagaagcagctggtggtcctggccgtggagcatacggccttggccctaatggagatcaaggtctgcgacggccaagagcacgtggtcaccgtctcgctgagggacggtgaggccaccctggaggtggacggcaccaggggccagagcgaggtgagcgccgcgcagctgcaggagaggctggccgtgctcgagaggcacctgcggagccccgtgctcacctttgctggcggcctgccagatgtgccggtgacttcagcgccagtcaccgcgttctaccgcggctgcatgacactggaggtcaaccggaggctgctggacctggacgaggcggcgtacaagcacagcgacatcacggcccactcctgcccccccgtggagcccgccgcagcctaggcccccacgggacgcggcaggcttctcagtctctgtccgagacagccgggaggagcctgggggctcctcaccacgtggggccatgctgagagctgggctttcctctgtgaccatcccggcctgtaacatatctgtaaatagtgagatggacttggggcctctgacgccgcgcactcagccgtgggcccgggcgcggggaggccggcgcagcgcagagcgggctcgaagaaaataattctctattatttttattaccaagcgcttctttctgactctaaaatatggaaaataaaatatttacagaaagctttgtaaaaaaaaaaaaaaaaaa

SEQ ID NO: 12 - protein, 678 aa, Homo sapiens
MAPSLSPGPAALRRAPQLLLLLLAAECALAALLPAREATQFLRPRQRRAFQVFEEAKQGHLERECVEELCSREEAREVFENDPETDYFYPRYLDCINKYGSPYTKNSGFATCVQNLPDQCTPNPCDRKGTQACQDLMGNFFCLCKAGWGGRLCDKDVNECSQENGGCLQICHNKPGSFHCSCHSGFELSSDGRTCQDIDECADSEACGEARCKNLPGSYSCLCDEGFAYSSQEKACRDVDECLQGRCEQVCVNSPGSYTCHCDGRGGLKLSQDMDTCEDILPCVPFSVAKSVKSLYLGRMFSGTPVIRLRFKRLQPTRLVAEFDFRTFDPEGILLFAGGHQDSTWIVLALRAGRLELQLRYNGVGRVTSSGPVINHGMWQTISVEELARNLVIKVNRDAVMKIAVAGDLFQPERGLYHLNLTVGGIPFHEKDLVQPINPRLDGCMRSWNWLNGEDTTIQETVKVNTRMQCFSVTERGSFYPGSGFAFYSLDYMRTPLDVGTESTWEVEVVAHIRPAADTGVLFALWAPDLRAVPLSVALVDYHSTKKLKKQLVVLAVEHTALALMEIKVCDGQEHVVTVSLRDGEATLEVDGTRGQSEVSAAQLQERLAVLERHLRSPVLTFAGGLPDVPVTSAPVTAFYRGCMTLEVNRRLLDLDEAAYKHSDITAHSCPPVEPAAA

SEQ ID NO: 13 - DNA, 2143 bp, Homo sapiens
actgaaggctagagaacaattccgagaaagagacggagagagagggaagaaaaagacagatagatagatattggggggaaggagaaaaaaggagaagagagggaagagaggacagcggagagagagcaccagagagagagggagagagagagagagcgctagagagagggagcgagcatgtgcgatgagcaatagctgtggaccttacagttgctgctaactgccctggtgtgtgtgagggagagagagggagggagggagagagagcgcgctagcgcgagagagcgagtgagcaagcgagcagaaaagaggtggagagggggggaataagaaagagagagaaggaaaggagagaaggcaggaagaaggcaagggacgagacaaccatgctgtgctgtatgagaagaaccaaacagaattaaaagggaacctggtctctgggttgttttcaacatctcaagtgtgaattttccctgtcaaaatcttcacaaggaaaatgagtcacagcatcacctgggtgacgaggtcataacacctcagcccttgcttaaaaaattttatttctacttttctattgtaaagagatctcaaaacaggaagataaaattggactgacagctctacagcctagtcttttagacagtgaactaggccagcattggcagacactggcgatgacaaagtcctgctctgaattatgccaccccgcactccactttttaccttgcctgggaggcttgaggaaaaatcttcagagagcagttcgacctagtccttattcacttggcttcttgactttctggatttcaagggttgaaaaaaatgatgacgaccaaaagattgaacaagatggtatcaaaccagaagataaagctcataaggccgcaaccaaaattcaggctagcttccgtggacacataacaaggaaaaagctcaaaggagagaagaaggatgatgtccaagctgctgaggctgaagctaataagaaggatgaagcccctgttgccgatggggtggagaagaagggagaaggcaccactactgccgaagcagccccagccactggctccaagcctgatgagcccggcaaagcaggagaaactccttccgaggagaagaagggggagggtgatgctgccacagagcaggcagccccccaggctcctgcatcctcagaggagaaggccggctcagctgagacagaaagtgccactaaagcttccactgataactcgccgtcctccaaggctgaagatgccccagccaaggaggagcctaaacaagccgatgtgcctgctgctgtcactgctgctgctgccaccacccctgccgcagaggatgctgctgccaaggcaacagcccagcctccaacggagactggggagagcagccaagctgaagagaacatagaagctgtagatgaaaccaaacctaaggaaagtgcccggcaggacgagggtaaagaagaggaacctgaggctgaccaagaacatgcctgaactctaagaaatggctttccacatccccaccctcccctctcctgagcctgtctctccctaccctcttctcagctccactctgaagtcccttcctgtcctgctcacgtctgtgagtctgtcctttcccacccactagccctctttctctctgtgtggcaaacatttaaaaaaaaaaaaaaaaagcaggaaagatcccaagtcaaacagtgtggcttaaacattttttgtttcttggtgttgttatggcaagtttttggtaatgatgattcaatcattttgggaaattcttgcactgtatccaagttatttgatctggtgcgtgtggccctgtgggagtccactttcctctctctctctctctctgttccaagtgtgtgtgcaatgttccgttcatctgaggagtccaaaatatcgagtgaattcaaaatcatttttgttttcctccttttcaatgtgatggaatgaacaaaaaggaaaaaattcaaaaaacccagtttgttttaaaaataaataaataaagcaaatgtgccaattagcgtaaacttgcggctctaaggctcctttttcaacccgaatattaataaatcatgagagtaatcaaggtcaaaaaaaaaaaaaaaaaaaaaaaaaaaaaaaaaaaaaaa

SEQ ID NO: 14 - protein, 274 aa, Homo sapiens
MTKSCSELCHPALHFLPCLGGLRKNLQRAVRPSPYSLGFLTFWISRVEKNDDDQKIEQDGIKPEDKAHKAATKIQASFRGHITRKKLKGEKKDDVQAAEAEANKKDEAPVADGVEKKGEGTTTAEAAPATGSKPDEPGKAGETPSEEKKGEGDAATEQAAPQAPASSEEKAGSAETESATKASTDNSPSSKAEDAPAKEEPKQADVPAAVTAAAATTPAAEDAAAKATAQPPTETGESSQAEENIEAVDETKPKESARQDEGKEEEPEADQEHA

SEQ ID NO: 15 - DNA, 5602 bp, Homo sapiens
atggagccctccagagcgcttctcggctgcctagcgagcgccgccgctgccgccccgccgggggaggatggagcaggggccggggccgaggaggaggaggaggaggaggaggaggcggcggcggcggtgggccccggggagctgggctgcgacgcgccgctgccctactggacggccgtgttcgagtacgaggcggcgggcgaggacgagctgaccctgcggctgggcgacgtggtggaggtgctgtccaaggactcgcaggtgtccggcgacgagggctggtggaccgggcagctgaaccagcgggtgggcatcttccccagcaactacgtgaccccgcgcagcgccttctccagccgctgccagcccggcggcgaggaccccagttgctacccgcccattcagttgttagaaattgattttgcggagctcaccttggaagagattattggcatcgggggctttgggaaggtctatcgtgctttctggataggggatgaggttgctgtgaaagcagctcgccacgaccctgatgaggacatcagccagaccatagagaatgttcgccaagaggccaagctcttcgccatgctgaagcaccccaacatcattgccctaagaggggtatgtctgaaggagcccaacctctgcttggtcatggagtttgctcgtggaggacctttgaatagagtgttatctgggaaaaggattcccccagacatcctggtgaattgggctgtgcagattgccagagggatgaactacttacatgatgaggcaattgttcccatcatccaccgcgaccttaagtccagcaacatattgatcctccagaaggtggagaatggagacctgagcaacaagattctgaagatcactgattttggcctggctcgggaatggcaccgaaccaccaagatgagtgcggcagggacgtatgcttggatggcacccgaagtcatccgggcctccatgttttccaaaggcagtgatgtgtggagctatggggtgctactttgggagttgctgactggtgaggtgccctttcgaggcattgatggcttagcagtcgcttatggagtggccatgaacaaactcgcccttcctattccttctacgtgcccagaaccttttgccaaactcatggaagactgctggaatcctgatccccactcacgaccatctttcacgaatatcctggaccagctaaccaccatagaggagtctggtttctttgaaatgcccaaggactccttccactgcctgcaggacaactggaaacacgagattcaggagatgtttgaccaactcagggccaaagaaaaggaacttcgcacctgggaggaggagctgacgcgggctgcactgcagcagaagaaccaggaggaactgctgcggcgtcgggagcaggagctggccgagcgggagattgacatcctggaacgggagctcaacatcatcatccaccagctgtgccaggagaagccccgggtgaagaaacgcaagggcaagttcaggaagagccggctgaagctcaaggatggcaaccgcatcagcctcccttctgatttccagcacaagttcacggtgcaggcctcccctaccatggataaaaggaagagtcttatcaacagccgctccagtcctcctgcaagccccaccatcattcctcgccttcgagccatccagttgacaccaggtgaaagcagcaaaacctggggcaggagctcagtcgtcccaaaggaggaaggggaggaggaggagaagagggccccaaagaagaagggacggacgtgggggccagggacgcttggtcagaaggagcttgcctcgggagatgaaggatcccctcagagacgtgagaaagctaatggtttaagtaccccatcagaatctccacatttccacttgggcctcaagtccctggtagatggatataagcagtggtcgtccagtgcccccaacctggtgaagggcccaaggagtagcccggccctgccagggttcaccagccttatggagatggccttgctggcagccagttgggtggtgcccatcgacattgaagaggatgaggacagtgaaggcccagggagtggagagagtcgcctacagcattcacccagccagtcctacctctgtatcccattccctcgtggagaggatggcgatggcccctccagtgatggaatccatgaggagcccaccccagtcaactcggccacgagtacccctcagctgacgccaaccaacagcctcaagcggggcggtgcccaccaccgccgctgcgaggtggctctgctcggctgtggggctgttctggcagccacaggcctagggtttgacttgctggaagctggcaagtgccagctgcttcccctggaggagcctgagccaccagcccgggaggagaagaaaagacgggagggtctttttcagaggtccagccgtcctcgtcggagcaccagccccccatcccgaaagcttttcaagaaggaggagcccatgctgttgctaggagacccctctgcctccctgacgctgctctccctctcctccatctccgagtgcaactccacacgctccctgctgcgctccgacagcgatgaaattgtcgtgtatgagatgccagtcagcccagtcgaggcccctcccctgagtccatgtacccacaaccccctggtcaatgtccgagtagagcgcttcaaacgagatcctaaccaatctctgactcccacccatgtcaccctcaccaccccctcgcagcccagcagtcaccggcggactccttctgatggggcccttaagccagagactctcctagccagcaggagcccctccagcaatgggttgagccccagtcctggagcaggaatgttgaaaacccccagtcccagccgagacccaggtgaattcccccgtctccctgaccccaatgtggtcttccccccaaccccaaggcgctggaacactcagcaggactctaccttggagagacccaagactctggagtttctgcctcggccgcgtccttctgccaaccggcaacggctggacccttggtggtttgtgtcccccagccatgcccgcagcacctccccagccaacagctccagcacagagacgcccagcaacctggactcctgctttgctagcagtagcagcactgtagaggagcggcctggacttccagccctgctcccgttccaggcagggccgctgcccccgactgagcggacgctcctggacctggatgcagaggggcagagtcaggacagcaccgtgccgctgtgcagagcggaactgaacacacacaggcctgccccttatgagatccagcaggagttctggtcttagcacgaaaaggattggggcgggcaagggggacagccagcggagatgaggggagctggcgggcacagccctttctcagggttggaccccctgagatccagccctacttcttgcactgataatgcactttgaagatggaagggatggaaacagggccacttcagagggtctcctgccctgcagggcctttctacccgtgtccactggaggggctgtggccatcagctctggctgtgtaggggaggaaggggtgcatgcatgtcccccaccctccacagtcttccttgcctttagagtgaccctgcagagtcactcagccaaatctgtctgctgctccctctcctcagccagttgggtgtgcgcagagctgtcatagggtccctttgtcagccccgagttcagcttcccaaacaccagtgttggatattctgtgattgattttggtcctcctccgctgtcccccaacacccaggaatgggaatctggcttggttcgagataggagcttttctgtgtcctaagccctttcatgctagcaggaagactgaaagcaaggtggcccagtgtggggtcatagggcttgatagacctggcactgcctatctgcacttccaggtgccccacctatttatctgagcccacaggtggaaaggggaactgcctcagtgagaacggggggacggggatgttaggaaaaatacagtaaagttgcaatgaagaggttcatgaagtatgtccttgttctttttggaaactctcggcaaagggcaaaccagcaagtattgagggtacccatctagctacttggggtcaggacctcgtcagaccaggttcggatacaatcatctgctcatcccaggaatagtttcttgggggactcactcactggtgccagttctaagtcagagacaaaattccactgtctgttccttttgctgtctgaactttatgtgttactcccttcctttggtcttcactctaatccctggagtttgtgggcttttggttatgtttggttagtagatatcaccgcaatgccctagaacagctatgaagcagaataccatatggccacctggacattgggacttgggaattcactctcaactgggccatccatgttgtgatgcccttgaagtaaaatggagccagcaggagtaccttctgtaaatgcatgtggcaaagtgctatttatagggtgcccagggagccgctgatgtacaataaccttgaggtcccccatactgaaaactgaccaaggcctgtgcacaggtagcccctcatgctgggctctggaccatgagctgagtaggaaggatagcagaggccaaccctgaccttcctggaagttgtttccttaacttgaatgttgagcttcctctaaagctttctcgtgtatgtcttctccatgccactactctgaggcctcctgtgttatgtgtgaacagttgtctttatgtgggaatgacgacttgattgggagtagagtctcaaggtcattcccctcttccctcaagactctctgaatgctgctccactgtcttttgtcttggaggtcactcagcaggttccttgcatttgctgcctggatgtgcagctggcaacagtgatgaattggtcactgctctttctctataactgggatagatgtcctgccttggggtcactaaaggggtgaccttgttccttgctttatgagcccattagcactttggttcaaggggcccaccaagtcttggacgggaaggcgctactggttttattgcccaaggttttgttattgcttctcttctgtgtccttctctttgttcagtgaagccaatatgtaagatactgtttttgtccccattcccctactcctgagctaggaggaaaaaatgtgaatcttaccagcagttccagccaaccaagtgattcttcttcattcttgatggggagaagtacatacaaagtttgttctgacagggcgcggtggctcacgcctgtaatcccagcgctttgggaggcagaggcaggtggatcacctgaggtcgggagttcgagaccagcctgaccaacatggagatatcctgtctctactaaaaatacaaaaaaattagccaggcatggtggcacgtgcctgtaatcccagctactcgcaaggctgaggcaggagaatcgcttgaacctgggaggcggaggttgcagtgagccaagattgcgccattgcactccagcctgggcaacaagagagaaactctgtctcaaaa

SEQ ID NO: 16 - protein, 1118 aa, Homo sapiens
MEPSRALLGCLASAAAAAPPGEDGAGAGAEEEEEEEEEAAAAVGPGELGCDAPLPYWTAVFEYEAAGEDELTLRLGDVVEVLSKDSQVSGDEGWWTGQLNQRVGIFPSNYVTPRSAFSSRCQPGGEDPSCYPPIQLLEIDFAELTLEEIIGIGGFGKVYRAFWIGDEVAVKAARHDPDEDISQTIENVRQEAKLFAMLKHPNIIALRGVCLKEPNLCLVMEFARGGPLNRVLSGKRIPPDILVNWAVQIARGMNYLHDEAIVPIIHRDLKSSNILILQKVENGDLSNKILKITDFGLAREWHRTTKMSAAGTYAWMAPEVIRASMFSKGSDVWSYGVLLWELLTGEVPFRGIDGLAVAYGVAMNKLALPIPSTCPEPFAKLMEDCWNPDPHSRPSFTNILDQLTTIEESGFFEMPKDSFHCLQDNWKHEIQEMFDQLRAKEKELRTWEEELTRAALQQKNQEELLRRREQELAEREIDILERELNIIIHQLCQEKPRVKKRKGKFRKSRLKLKDGNRISLPSDFQHKFTVQASPTMDKRKSLINSRSSPPASPTIIPRLRAIQLTPGESSKTWGRSSVVPKEEGEEEEKRAPKKKGRTWGPGTLGQKELASGDEGSPQRREKANGLSTPSESPHFHLGLKSLVDGYKQWSSSAPNLVKGPRSSPALPGFTSLMEMALLAASWVVPIDIEEDEDSEGPGSGESRLQHSPSQSYLCIPFPRGEDGDGPSSDGIHEEPTPVNSATSTPQLTPTNSLKRGGAHHRRCEVALLGCGAVLAATGLGFDLLEAGKCQLLPLEEPEPPAREEKKRREGLFQRSSRPRRSTSPPSRKLFKKEEPMLLLGDPSASLTLLSLSSISECNSTRSLLRSDSDEIVVYEMPVSPVEAPPLSPCTHNPLVNVRVERFKRDPNQSLTPTHVTLTTPSQPSSHRRTPSDGALKPETLLASRSPSSNGLSPSPGAGMLKTPSPSRDPGEFPRLPDPNVVFPPTPRRWNTQQDSTLERPKTLEFLPRPRPSANRQRLDPWWFVSPSHARSTSPANSSSTETPSNLDSCFASSSSTVEERPGLPALLPFQAGPLPPTERTLLDLDAEGQSQDSTVPLCRAELNTHRPAPYEIQQEFWS

SEQ ID NO: 17 - DNA, 20 bp, Artificial Sequence
  Description of Artificial Sequence Synthetic primer
ttactcgccacaagatgctg

SEQ ID NO: 18 - DNA, 20 bp, Artificial Sequence
  Description of Artificial Sequence Synthetic primer
gcaccatgccataaacattg

SEQ ID NO: 19 - DNA, 20 bp, Artificial Sequence
  Description of Artificial Sequence Synthetic primer
aacatcatccctgcttccac

SEQ ID NO: 20 - DNA, 20 bp, Artificial Sequence
  Description of Artificial Sequence Synthetic primer
gaccacctggtcctcagtgt